(19) United States
(12) Patent Application Publication
(10) Pub. No.: US 2023/0220068 A1
(43) Pub. Date: Jul. 13, 2023

(54) ANTI-TRANSFERRIN EXTRACELLULAR VESICLES

(71) Applicants: Ajay VERMA, Cambridge, MA (US); Leonid GAYDUKOV, Cambridge, MA (US); Codiak BioSciences, Inc., Cambridge, MA (US)

(72) Inventors: Ajay VERMA, Wayland, MA (US); Leonid GAYDUKOV, Tewksbury, MA (US); Ke XU, Belmont, MA (US); Kevin P. DOOLEY, Boston, MA (US); Jonathan FINN, Melrose, MA (US)

(73) Assignee: Codiak BioSciences, Inc., Cambridge, MA (US)

(21) Appl. No.: 18/000,799

(22) PCT Filed: Jun. 7, 2021
(86) PCT No.: PCT/US2021/036234

§ 371 Date: Dec. 5, 2022

Related U.S. Application Data
(60) Provisional application No. 63/161,362, filed on Mar. 15, 2021.
(60) Provisional application No. 63/035,370, filed on Jun. 5, 2020.

(51) Int. Cl. C07K 16/28; C07K 14/54

(52) U.S. Cl. CPC C07K 16/28; C07K 14/5434; C07K 2317/22; C07K 2317/92

(57) ABSTRACT

The present disclosure relates extracellular vesicles, e.g., exosomes, comprising an antigen-binding moeity, e.g., a single-domain antigen-binding moeity, e.g., a vNAR, a VHH, or a fragment thereof, that specifically binds transferrin receptor, and methods of making and using the same.

### REFERENCE TO SEQUENCE LISTING SUBMITTED ELECTRONICALLY VIA EFS-WEB

The content of the electronically submitted sequence listing (Name: 4000_100PC02_Seqlisting_ST25, Size: 82,323 bytes; and Date of Creation: Jun. 4, 2021) submitted in this application is incorporated herein by reference in its entirety.

### CROSS REFERENCE TO RELATED APPLICATIONS

This PCT application claims the priority benefit of U.S. Provisional Application Nos. 63/035,370 filed Jun. 5, 2020; and 63/161,362 filed Mar. 15, 2021, each of which is incorporated herein by reference in its entirety.

### FIELD OF DISCLOSURE

The present disclosure relates to targeted delivery of modified extracellular vesicles (EVs) (e.g., exosomes), and the use of such EVs to treat and/or prevent a range of medical disorders, such as diseases that affect the central nervous system.

### BACKGROUND OF DISCLOSURE

EVs (e.g., exosomes) are important mediators of intercellular communication. They are also important biomarkers in the diagnosis and prognosis of many diseases, such as cancer. As drug delivery vehicles, EVs offer many advantages over traditional drug delivery methods (e.g., peptide immunization, DNA vaccines) as a new treatment modality in many therapeutic areas. However, despite its advantages, many EVs have had limited clinical efficacy. For example, dendritic-cell derived exosomes (DEX) were investigated in a Phase II clinical trial as maintenance immunotherapy after first line chemotherapy in patients with inoperable non-small cell lung cancer (NSCLC). However, the trial was terminated because the primary endpoint (at least 50% of patients with progression-free survival (PFS) at 4 months after chemotherapy cessation) was not reached. Besse, B., et al., Oncoimmunology 5(4):e1071008 (2015).

Targeting of EVs to a particular cell or tissue can be achieved, for example, by loading an antibody or a fragment thereof on the surface of the EV. However, antibodies, and even scFv fragments of antibodies, are very large and difficult to express and/or localize at a high concentration on the surface of EVs.

Accordingly, new and more effective engineered-EVs, particularly those that can specifically target specific tissues, are necessary to better enable therapeutic use and other applications of EV-based technologies.

### SUMMARY OF DISCLOSURE

Certain aspects of the present disclosure are directed to an extracellular vesicle (EV) comprising an antigen-binding moiety that specifically binds transferrin receptor (TfR), wherein the antigen-binding moiety is loaded on the exterior surface of the EV. In some aspects, the antigen-binding moiety specifically binds human TfR.

In some aspects, the antigen-binding moiety increases the transport of the EV across the blood brain barrier in a human subject. In some aspects, the antigen-binding moiety increases the transport of the EV across the blood brain barrier by at least about 10%, at least about 20%, at least about 30%, at least about 40%, at least about 75%, at least about 100%, at least about 150%, at least about 200%, at least about 250%, at least about 300%, at least about 350%, at least about 400%, at least about 450%, at least about 500%, at least about 600%, at least about 700%, at least about 800%, at least about 900%, or at least about 1000%, as compared to a reference EV not comprising an antigen-binding moiety that specifically binds human TfR.

In some aspects, the antigen-binding moiety an antibody or an antigen-binding portion thereof. In some aspects, the antigen-binding moiety comprises one or more single-domain antigen-binding moieties. In some aspects, the antigen-binding moiety comprises at least two single-domain antigen-binding moieties. In some aspects, the at least two single-domain antigen-binding moieties are the same. In some aspects, the at least two single-domain antigen-binding moieties are different. In some aspects, the at least two single-domain antigen-binding moieties comprise (i) a first single-domain antigen-binding moiety that binds a first epitope on TfR and (ii) a second single-domain antigen-binding moiety binds a second epitope on TfR. In some aspects, the antigen-binding moiety comprises at least three, at least four, at least five, or at least 6 single-domain antigen-binding moieties. In some aspects, each of the one or more single-domain antigen-binding moieties are linked to each other by a linker.

In some aspects, the one or more single-domain antigen-binding moieties are selected from a VHH, a vNAR, an antigen-binding fragment of a VHH, an antigen-binding fragment of a vNAR, and any combination thereof. In some aspects, the vNAR or the fragment thereof is derived from an IgNAR. In some aspects, the vNAR or the fragment thereof is synthetic. In some aspects, the VHH or the fragment thereof is derived from a camelid antibody. In some aspects, the VHH or the fragment thereof is synthetic.

In some aspects, the single-domain antigen-binding moiety increases the permeability of the EV across the blood brain barrier by at least about 10%, at least about 20%, at least about 30%, at least about 40%, at least about 75%, at least about 100%, at least about 150%, at least about 200%, at least about 250%, at least about 300%, at least about 350%, at least about 400%, at least about 450%, at least about 500%, at least about 600%, at least about 700%, at least about 800%, at least about 900%, or at least about 1000%, as compared to an EV comprising an IgG antibody or a fragment thereof that specifically binds human TfR. In some aspects, the single-domain antigen-binding moiety increases the permeability of the EV across the blood brain barrier by at least about 10%, at least about 20%, at least about 30%, at least about 40%, at least about 75%, at least about 100%, at least about 150%, at least about 200%, at least about 250%, at least about 300%, at least about 350%, at least about 400%, at least about 450%, at least about 500%, at least about 600%, at least about 700%, at least about 800%, at least about 900%, or at least about 1000%, as compared to an EV comprising an scFv that specifically binds human TfR.

In some aspects, the antigen-binding moiety is present at a concentration of at least about 10 copies per EV, at least about 20 copies per EV, at least about 30 copies per EV, at least about 40 copies per EV, at least about 50 copies per EV, at least about 75 copies per EV, at least about 100 copies per EV, at least about 150 copies per EV, at least about 200 copies per EV, at least about 250 copies per EV, at least about 300 copies per EV, at least about 350 copies per EV, at least about 400 copies per EV, at least about 450 copies per EV, at least about 500 copies per EV, at least about 600 copies per EV, at least about 700 copies per EV, at least about 800 copies per EV, at least about 900 copies per EV, at least about 1000 copies per EV, at least about 1250 copies per EV, at least about 1500 copies per EV, at least about 2000 copies per EV, at least about 2500 copies per EV, at least about 3000 copies per EV, at least about 3500 copies per EV, at least about 4000 copies per EV, at least about 4500 copies per EV, or at least about 5000 copies per EV. In some aspects, the antigen-binding moiety is present at a concentration of at least about 500 copies per EV. In some aspects, the antigen-binding moiety is present at a concentration of at least about 1000 copies per EV.

In some aspects, the antigen-binding moiety specifically binds human TfR with a K_{D} of less than 5×10⁻⁶, 2×10⁻⁶, 1×10⁻⁶, 5×10⁻⁷, 1×10⁻⁷, 5×10⁻⁸, 1×10⁻⁸, 5×10⁻⁹, or 1×10⁻⁹M.

In some aspects, the EV further comprises an anti-phagocytic signal. In some aspects, the anti-phagocytic signal is selected from CD47, CD24, a fragment thereof, and any combination thereof. In some aspects, the anti-phagocytic signal is associated with the exterior surface of the EV.

In some aspects, the EV further comprises a biologically active moiety. In some aspects, the biologically active moiety comprises a therapeutic molecule, immune modulator, adjuvant, or any combination thereof or a nucleic acid encoding the therapeutic molecule, immune modulator, adjuvant, or any combination thereof. In some aspects, the nucleic acid encoding the therapeutic molecule, immune modulator, adjuvant, or any combination thereof comprises an mRNA, siRNA, shRNA, miRNA, or any combination thereof. In some aspects, the therapeutic molecule comprises an antigen. In some aspects, the adjuvant comprises a Stimulator of Interferon Genes (STING) agonist, a toll-like receptor (TLR) agonist, an inflammatory mediator, or any combination thereof. In some aspects, the adjuvant comprises a STING agonist. In some aspects, the STING agonist comprises a cyclic dinucleotide STING agonist or a non-cyclic dinucleotide STING agonist. In some aspects, the adjuvant is a TLR agonist. In some aspects, the TLR agonist comprises a TLR2 agonist (e.g., lipoteichoic acid, atypical LPS, MALP-2 and MALP-404, OspA, porin, LcrV, lipomannan, GPI anchor, lysophosphatidylserine, lipophosphoglycan (LPG), glycophosphatidylinositol (GPI), zymosan, hsp60, gH/gL glycoprotein, hemagglutinin), a TLR3 agonist (e.g., double-stranded RNA, e.g., poly(I:C)), a TLR4 agonist (e.g., lipopolysaccharides (LPS), lipoteichoic acid, β-defensin 2, fibronectin EDA, HMGB1, snapin, tenascin C), a TLR5 agonist (e.g., flagellin), a TLR6 agonist, a TLR7/8 agonist (e.g., single-stranded RNA, CpG-A, Poly G10, Poly G3, Resiquimod), a TLR9 agonist (e.g., unmethylated CpG DNA), or any combination thereof. In some aspects, the immune modulator comprises a cytokine. In some aspects, the cytokine comprises IL-12.

In some aspects, the antigen-binding moiety, the biologically active moiety, and/or the anti-phagocytic signal are linked to the exterior surface of the EV by a scaffold protein. In some aspects, the scaffold protein is a Scaffold X protein. In some aspects, the Scaffold X protein comprises prostaglandin F2 receptor negative regulator (the PTGFRN protein); basigin (the BSG protein); immunoglobulin superfamily member 2 (the IGSF2 protein); immunoglobulin superfamily member 3 (the IGSF3 protein); immunoglobulin superfamily member 8 (the IGSF8 protein); integrin beta-1 (the ITGB1 protein); integrin alpha-4 (the ITGA4 protein); 4F2 cell-surface antigen heavy chain (the SLC3A2 protein); a class of ATP transporter proteins (the ATP1A1, ATP1A2, ATP1A3, ATP1A4, ATP1B3, ATP2B1, ATP2B2, ATP2B3, ATP2B4 proteins), CD13, aminopeptidase N (ANPEP), neprilysin (membrane metalloendopeptidase; MME), ectonucleotide pyrophosphatase/phosphodiesterase family member 1 (ENPP1), neuropilin-1 (NRP1), CD9, CD63, CD81, PDGFR, GPI anchor proteins, lactadherin, LAMP2, LAMP2B, a fragment thereof, or any combination thereof. In some aspects, the Scaffold X protein comprises the amino acid sequence set forth as SEQ ID NO: 33. In some aspects, the Scaffold X protein comprises an amino acid sequence having at least about 70%, at least about 75%, at least about 80%, at least about 85%, at least about 90%, at least about 95%, at least about 96%, at least about 97%, at least about 98%, at least about 99%, or about 100% sequence identity to SEQ ID NO: 1.

In some aspects, the biologically active moiety is linked to the luminal surface of the EV by a scaffold protein. In some aspects, the scaffold protein is a Scaffold Y protein. In some aspects, the Scaffold Y protein comprises myristoylated alanine rich Protein Kinase C substrate (the MARCKS protein), myristoylated alanine rich Protein Kinase C substrate like 1 (the MARCKSL1 protein), brain acid soluble protein 1 (the BASP1 protein), a fragment thereof, and or any combination thereof. In some aspects, the Scaffold Y protein is BASP1 protein or a fragment thereof.

In some aspects, the Scaffold Y protein comprises an N-terminus domain (ND) and an effector domain (ED), wherein the ND and/or the ED are associated with the luminal surface of the EV. In some aspects, the ND is associated with the luminal surface of the EV via myristoylation. In some aspects, the ED is associated with the luminal surface of the EV by an ionic interaction. In some aspects, the ED comprises (i) a basic amino acid or (ii) two or more basic amino acids in sequence, wherein the basic amino acid is selected from the group consisting of Lys, Arg, His, and any combination thereof. In some aspects, the basic amino acid is (Lys)n, wherein n is an integer between 1 and 10. In some aspects, the ED comprises Lys (K), KK, KKK, KKKK (SEQ ID NO: 205), KKKKK (SEQ ID NO: 206), Arg (R), RR, RRR, RRRR (SEQ ID NO: 207); RRRRR (SEQ ID NO: 208), KR, RK, KKR, KRK, RKK, KRR, RRK, (K/R)(K/R)(K/R)(K/R) (SEQ ID NO: 209), (K/R)(K/R)(K/R)(K/R)(K/R) (SEQ ID NO: 210), or any combination thereof. In some aspects, the ND comprises an amino acid sequence selected from the group consisting of (i) GGKLSKK (SEQ ID NO: 211), (ii) GAKLSKK (SEQ ID NO: 212), (iii) GGKQSKK (SEQ ID NO: 213), (iv) GGKLAKK (SEQ ID NO: 214), and (vi) any combination thereof. In some aspects, the scaffold protein comprises (i) GGKLSKKKKGYNVN (SEQ ID NO: 246), (ii) GAKLSKKKKGYNVN (SEQ ID NO: 247), (iii) GGKQSKKKKGYNVN (SEQ ID NO: 248), (iv) GGKLAKKKKGYNVN (SEQ ID NO: 249), (v) GGKLSKKKKGYSGG (SEQ ID NO: 250), (vi) GGKLSKKKKGSGGS (SEQ ID NO: 251), (vii) GGKLSKKKKSGGSG (SEQ ID NO: 252), (viii) GGKLSKKKSGGSGG (SEQ ID NO: 253), (ix) GGKLSKKSGGSGGS (SEQ ID NO: 254), (x) GGKLSKSGGSGGSV (SEQ ID NO: 255), or (xi) GAKKSKKRFSFKKS (SEQ ID NO: 256).

In some aspects, the EV is an exosome, a microvesicle, an apoptotic body, or any combination thereof. In some aspects, the EV is an exosome.

Certain aspects of the present disclosure are directed to a pharmaceutical composition comprising an EV disclosed herein and a therapeutic molecule, an immune modulator, an adjuvant, or any combination thereof. In some aspects, the therapeutic molecule comprises an antigen. In some aspects, the adjuvant comprises a Stimulator of Interferon Genes (STING) agonist, a toll-like receptor (TLR) agonist, an inflammatory mediator, or any combination thereof. In some aspects, the adjuvant comprises a STING agonist. In some aspects, the STING agonist comprises a cyclic dinucleotide STING agonist or a non-cyclic dinucleotide STING agonist. In some aspects, the adjuvant is a TLR agonist. In some aspects, the TLR agonist comprises a TLR2 agonist (e.g., lipoteichoic acid, atypical LPS, MALP-2 and MALP-404, OspA, porin, LcrV, lipomannan, GPI anchor, lysophosphatidylserine, lipophosphoglycan (LPG), glycophosphatidylinositol (GPI), zymosan, hsp60, gH/gL glycoprotein, hemagglutinin), a TLR3 agonist (e.g., double-stranded RNA, e.g., poly(I:C)), a TLR4 agonist (e.g., lipopolysaccharides (LPS), lipoteichoic acid, β-defensin 2, fibronectin EDA, HMGB1, snapin, tenascin C), a TLR5 agonist (e.g., flagellin), a TLR6 agonist, a TLR7/8 agonist (e.g., single-stranded RNA, CpG-A, Poly G10, Poly G3, Resiquimod), a TLR9 agonist (e.g., unmethylated CpG DNA), or any combination thereof. In some aspects, the therapeutic molecule, the immune modulator, the adjuvant, or any combination thereof, is associated with Scaffold X, Scaffold Y, or a combination thereof. In some aspects, the immune modulator comprises a cytokine. In some aspects, the cytokine comprises an interferon. In some aspects, pharmaceutical compositions further comprises a pharmaceutically acceptable carrier.

Certain aspects of the present disclosure are directed to a method of treating a disease or disorder in a subject in need thereof comprising administering to the subject an EV disclosed herein or a pharmaceutical composition disclosed herein. In some aspects, the disease or disorder is a neurological disease or disorder. In some aspects, the neurological disease or disorder comprises a tumor. In some aspects, the neurological disease or disorder comprises an acoustic neuroma, choroid plexus carcinoma, craniopharyngioma, embryonal tumor, glioma, medulloblastoma, meningioma, pediatric brain tumor, pineoblastoma, pituitary tumor, or a combination thereof. In some aspects, the glioma is selected from an ependymoma, astrocytoma, oligodendroglioma, brainstem glioma, optic nerve glioma, mixed glioma, oligoastrocytoma, or any combination thereof. In some aspects, the astrocytoma comprises glioblastoma multiforme (GBM). In some aspects, the disease or disorder comprises a neoplastic meningitis, Parkinson disease, Alzheimer Disease, Huntington Disease, amyotrophic lateral sclerosis (ALS), or any combination thereof.

Certain aspects of the present disclosure are directed to a method of targeting an extracellular vesicle (EV) to a cell of the central nervous system, comprising loading an antigen-binding moiety that specifically binds transferrin receptor (TfR) on the surface of the extracellular vesicle. Certain aspects of the present disclosure are directed to a method of increasing the permeability of an EV across the blood brain barrier in a human subject, comprising loading on the surface of the EV an antigen-binding moiety that specifically binds TfR. In some aspects, the antigen-binding moiety specifically binds human TfR. In some aspects, the antigen-binding moiety increases the transport of the EV across the blood brain barrier in a human subject. In some aspects, the antigen-binding moiety increases the transport of the EV across the blood brain barrier by at least about 10%, at least about 20%, at least about 30%, at least about 40%, at least about 75%, at least about 100%, at least about 150%, at least about 200%, at least about 250%, at least about 300%, at least about 350%, at least about 400%, at least about 450%, at least about 500%, at least about 600%, at least about 700%, at least about 800%, at least about 900%, or at least about 1000%, as compared to a reference EV not comprising an antigen-binding moiety that specifically binds human TfR.

In some aspects, the antigen-binding moiety an antibody or an antigen-binding portion thereof. In some aspects, the antigen-binding moiety comprises one or more single-domain antigen-binding moieties. In some aspects, the antigen-binding moiety comprises at least two single-domain antigen-binding moieties. In some aspects, the at least two single-domain antigen-binding moieties are the same. In some aspects, the at least two single-domain antigen-binding moieties are different. In some aspects, the at least two single-domain antigen-binding moieties comprise (i) a first single-domain antigen-binding moiety that binds a first epitope on TfR and (ii) a second single-domain antigen-binding moiety binds a second epitope on TfR. In some aspects, the antigen-binding moiety comprises at least three, at least four, at least five, or at least six single-domain antigen-binding moieties. In some aspects, each of the one or more single-domain antigen-binding moieties are linked to each other by a linker.

In some aspects, the one or more single-domain antigen-binding moieties are selected from a VHH, a vNAR, an antigen-binding fragment of a VHH, an antigen-binding fragment of a vNAR, and any combination thereof. In some aspects, the vNAR or the fragment thereof is derived from an IgNAR. In some aspects, the vNAR or the fragment thereof is synthetic. In some aspects, the VHH or the fragment thereof is derived from a camelid antibody. In some aspects, the VHH or the fragment thereof is synthetic.

### BRIEF DESCRIPTION OF THE DRAWINGS

FIGS. 1A-1D are schematic drawings of various CD47-Scaffold X fusion constructs.

FIG. 1A shows constructs comprising the extracellular domain of wild-type CD47 (with a C15S substitution) fused to either a flag-tagged (1083 and 1084) or non-flag-tagged (1085 and 1086) full length Scaffold X (1083 and 1086) or a truncated Scaffold X (1084 and 1085). FIG. 1B shows constructs comprising the extracellular domain of Velcro-CD47 fused to either a flag-tagged (1087 and 1088) or non-flag-tagged (1089 and 1090) full length Scaffold X (1087 and 1090) or a truncated Scaffold X (1088 and 1089). FIG. 1C shows constructs wherein the first transmembrane domain of wild-type CD47 (with a C15S substitution; 1127 and 1128) or Velcro-CD47 (1129 and 1130) is replaced with a fragment of Scaffold X, comprising the transmembrane domain and the first extracellular motif of Scaffold X. FIG. 1D shows various constructs comprising a minimal “self” peptide (GNYTCEVTELTREGETIIELK; SEQ ID NO: 160) fused to either a flag-tagged (1158 and 1159) or non-flag-tagged (1160 and 1161) full length Scaffold X (1158 and 1161) or a truncated Scaffold X (1159 and 1160).

FIGS. 2A-2B are graphical representations of CD47 expression on exosomes as measured by ELISA (FIG. 2A) and by a SIRPα signaling reporter assay (FIG. 2B). FIG. 2A is a bar graph showing the concentration of CD47 molecules on the surface of exosomes expressing each of the constructs in FIGS. 1A-1D, as measured by ELISA. FIG. 2B is a bar graph showing the relative concentration CD47 molecules on the surface of exosomes as measured by the chemiluminescence generated in a SIRPα signaling reporter assay (DiscoverX).

FIGS. 3A-3C are graphical representations illustrating SIRPα binding by each of the CD47-Scaffold X constructs, expressed on exosomes, as measured by Octet assay. All steps are aligned by step baseline (sensor location).

FIGS. 4A-4D show the uptake of GFP-Scaffold-Xexosomes by primary human monocyte-derived M0 macrophages. FIGS. 4A-4C are cell images showing IncuCyte real-time analysis for GFP-Scaffold-X surface expressing exosome localization to primary human monocyte-derived M0 macrophages as an overlay (FIG. 4A), a confluence mask (FIG. 4B), and a fluorescence mask (FIG. 4C). FIG. 4D is a graphical illustration of localization of GFP positive exosomes to primary human monocyte-derived M0 macrophages over time following exposure to exosomes at a dose of 4.4×10⁸ particles/mL exosomes (triangles), 1.33×10⁹ particles/mL exosomes (squares), and 4×10⁹ particles/mL exosomes (circles). FIGS. 4E-4L are graphical representations of localization of CD47 surface-expressing exosomes to primary human monocyte-derived M0 macrophages (FIGS. 4E-4G) or HEK cells (FIGS. 4H-41) over time following exposure of the cells to exosomes at a concentration of 4×10⁹ particles/mL (FIG. 4E), 1.33×10⁹ particles/mL (FIG. 4F), 4.4×10⁸ particles/mL exosomes (FIG. 4G), 5×10¹⁰ particles/mL FIGS. 41), and 1.67×10¹⁰ particles/mL (FIG. 4H).

FIGS. 5A-5B show the expression of various mouse CD47-Scaffold X fusion constructs (FIG. 5A) on the surface of modified exosomes. FIG. 5A shows constructs comprising the extracellular domain of wild-type murine CD47 (with a C15S substitution) fused to either a flag-tagged (1923 and 1925) or non-flag-tagged (1924 and 1922) full length Scaffold X (1923 and 1922) or a truncated Scaffold X (1925 and 1924). FIG. 5B is a graphical representation showing the number of murine CD47 particles localizing to the surface of exosomes.

FIG. 6 is a graphical representation illustrating binding of both human and mouse CD47 exosomes to mouse SIRPα, as measured by Octet assay.

FIGS. 7A-7B are graphical illustrations of localization of the various mouse CD47-Scaffold X fusion constructs (FIG. 5A) in mouse bone marrow-derived macrophages over time following exposure of the macrophages to exosomes at a concentration of 1.67×10¹⁰ particles/mL (FIGS. 7A-7B).

FIGS. 8A-8N show representative images of exosome localization in mouse bone marrow-derived macrophages exposed to 5×10¹⁰ particles/mL of Scaffold X modified exosomes (FIGS. 8A and 8B), native exosomes (FIGS. 8C and 8D), exosomes expressing the extracellular domain of murine CD47^{C15S} fused to Scaffold X (FIGS. 8E and 8F), and exosomes expressing the extracellular domain of murine CD47^{C15S} fused to a flag-tagged Scaffold X (FIGS. 8G and 8H), at 2 hours (FIGS. 8A, 8C, 8E, and 8G), 7.5 hours (FIGS. 8B, 8D, 8F, and 8H), and 19.5 hours (FIGS. 8K-8N). FIGS. 8I and 8J show localization of native exosomes to HEKsf cells.

FIGS. 9A-9D show exoRVG uptake in neuro2A cells. The constructs tested were: RVG-PrX-mCherry-FLAG-HiBiT (construct 2021; FIG. 9A), linker-PrX-mCherry-FLAG-HiBiT (construct 2022; FIG. 9B), RVG-LAMP2B-mCherry-FLAG-HiBiT (construct 2023; FIG. 9C), and linker-LAMP2B-mCherry-FLAG-HiBiT (construct 2024; FIG. 9D). Only the constructs comprising RVG showed uptake by the neuro2A cells. 10⁵ EV particles per cell were used. EV uptake was observed at 5 hours. “RVG” is a tropism moiety of sequence YTIWMPENPRPGTPCDIFTNSRGKRASNG (SEQ ID NO: 165). “Linker” is a linker of sequence GGSSGSGSGSGGGGSGGGGTGTSSSGTGT (SEQ ID NO: 192). “FLAG” is a FLAG® epitope tag. “HiBiT” is the nano luciferase peptide described above. “mCherry” is a red fluorescent protein. “LAMP2B” and “PrX” are protein scaffolds. “ExoRVG” EV are exosomes comprising an RVG tropism moiety.

FIGS. 10A-10B show exoRVG uptake in neuro2A cells 18 hours after the cells were incubated with 5×10⁴ EV particles per neuro2A cell. Measurements were taken 18 hours after uptake. The constructs tested were exoRVG (construct 2021, see FIG. 9) (FIG. 10A) and exoLinker (construct 2020, see FIG. 9) (FIG. 10B). Uptake was only observed when constructs comprising the tropism moiety RVG were used,

FIGS. 11A-11X show exoRVG uptake in neuro2A cells 24 hours after incubation with EV comprising one of the four constructs described in FIG. 9. Samples used were negative control (no EV particles; FIGS. 11A-11D), E5 (10⁵ particles/cell; FIGS. 11E-11H), 5E4 (5×10⁴ particles/cell; FIGS. 11I-11L), E4 (10⁴ particles/cell; FIGS. 11M-11P), 5E3 (5×10³ particles/cell; FIGS. 11O-11T), and E3 (10³ particles/cell; FIGS. 11U-11X). The boxed data sets corresponds to the samples used in FIG. 10 measured at 24 hours after uptake.

FIG. 12 compares EV uptake in neuro2A cells corresponding to negative control (leftmost curve), exoLinker (construct 2020) (center curve), and exoRVG (construct 2021) (rightmost curve), measured 24 hours after the cells were incubated with 5×10⁴ EV particles per neuro2A cell.

FIGS. 13A-13C show exoTransferrin uptake in HeLa cells. Three constructs were tested: Transferrin-PrX-mCherry-FLAG (human transferrin; construct 1597; FIG. 13A), mTransferrin-PrX-mCherry-FLAG (mouse transferrin; construct 1598; FIG. 13B); and linker-PrX-mCherry-FLAG-HiBiT (construct 2022; FIG. 13C). 5×10⁵ EV particles per HeLa cell were used. “ExoTransferrin” EV are exosomes comprising a transferrin tropism moiety. Uptake was measured 3 hours after EV particle incubation started. EV uptake was observed for both human and mouse transferrin-containing EVs.

FIGS. 14A-14C show exoTransferrin uptake in Hep3B cells. Three constructs were tested: Transferrin-PrX-mCherry-FLAG (human transferrin; construct 1597; FIG. 14A), mTransferrin-PrX-mCherry-FLAG (mouse transferrin; construct 1598; FIG. 14B); and linker-PrX-mCherry-FLAG-HiBiT (construct 2022; FIG. 14C). 5×10⁵ EV particles per Hep3B cell were used. Uptake was measured 3 hours after EV particle incubation started. EV uptake was observed for both human and mouse transferrin-containing EVs.

FIGS. 15A-15C show exoTransferrin uptake in Hep3G2 cells. Three constructs were tested: Transferrin-PrX-mCherry-FLAG (human transferrin; construct 1597; FIG. 15A), mTransferrin-PrX-mCherry-FLAG (mouse transferrin; construct 1598; FIG. 15B); and linker-PrX-mCherry-FLAG-HiBiT (construct 2022; FIG. 15C). 5×10⁵ EV particles per HepG2 cell were used. Uptake was measured 3 hours after EV particle incubation started. EV uptake was observed for both human and mouse transferrin-containing EVs.

FIG. 16A shows a schematic diagram of exemplary extracellular vesicle targeting Trks using neurotrophin-Scaffold X fusion construct that can be delivered along with a STING agonist. Neurotrophins bind to Trk receptors as a homo dimer and allow the EV to target a sensory neuron.

FIG. 16B shows a schematic diagram of exemplary extracellular vesicle having (i) neuro-tropism as well as (ii) an anti-phagocytic signal, e.g., CD47 and/or CD24, on the exterior surface of the EV that can be delivered along with (iii) an anti-TfR binding moiety disclosed herein.

FIGS. 17A-171 are fluorescent images of neuro2A cells cultuted in the presense of ExoTransferrin (1597; FIGS. 17A, 17D, and 17G), ExoTransferrin (1598; FIGS. 17B, 17E, and 17H), or ExoLinker (negative control; FIGS. 17C, 17F, and 17I).

FIGS. 18A-18C are fluorescent images of neuro2A cells cultuted in the presense of ExoTransferrin, showing Th expression (FIG. 18A) and overlay of TH expression and mCherry tagged EVs (FIGS. 18B-18C).

FIGS. 19A-19B are fluorescent images of human neuroblastoma cells (SH-SY-5Y) incubated with EV samples comprising a control (exoLinker; FIG. 19A) or exo-mTransferrin (FIG. 19B).

FIGS. 20A-20I are fluorescent images of primary mouse Schwann cells cells cultuted in the presense of exoTransferrin (1597; FIGS. 20A, 20D, and 20G), exo-mTransferrin (1598; FIGS. 20B, 20E, and 20H), or ExoLinker (negative control; FIGS. 20C, 20F, and 20I) at 2 hours (FIGS. 20A-20C), 5 hours (FIGS. 20D-20F) and 22 hours (FIGS. 20G-20I).

FIGS. 21A-21I are fluorescent images of primary human Schwann cells cells cultuted in the presense of exoTransferrin (1597; FIGS. 21A, 21D, and 21G), exo-mTransferrin (1598; FIGS. 21B, 21E, and 21H), or ExoLinker (negative control; FIGS. 21C, 21F, and 21I) at 2 hours (FIGS. 21A-21C), 5 hours (FIGS. 21D-21F) and 22 hours (FIGS. 21G-211).

FIGS. 22A-22B are images of primary mouse Schwann cells (FIG. 22A) and primary human Schwann cells (FIG. 22B), which were cultured in the presence of exoTransferrin, fixed, and stained with anti-cytoskeleton-marker antibody and DAPI.

FIGS. 23A-23B are fluorescence images of SH-SY-5Y cells cultuted in the presense of EVs expressing PrX-GFP (negative control; FIG. 23) or anti-TfnR(8D3)-PrX-GFP (FIG. 23B) overnight. FIG. 23C is a bar graph showing the transferrin copy number per EV particle for exoTransferrin (1597) small scale and large scale and exo-mTransferrin (1598) small scale and large scale.

FIG. 24 shows a PMP22 knockdown assay in mouse Schwann cells comprising the administration of ExomTF-PMP22 ASO contructs, i.e., exosomes expressing mouse transferrin on their surfaces and also comprising an ASO against PMP22. ASOs were administered at 125 nM, 250 nM, and 500 nM concentrations. Exosomes comprising the scrambled sequence of the PMP22 ASO were used as controls. Other controls comprised Schwann cells (“cells only”), Lipofectamine RNAiMAX (“RNAimax only”), PMP22 ASO alone (“Free PMP22 ASO”), exomTransferrin (mouse Transferrin) (“1598 exo only”), samples transfected with PMP22 ASO with RNAiMAX (“PMP22trans”), and samples transfected with control ASO with RNAiMAX (“controltrans”). The ASO used was X61832, i.e., a PMP22 ASO of sequence CTCATTCGCGTTTCCGC (SEQ ID NO: 146).

FIGS. 25A-25D show uptake of PTGFRN overexpressing (PrX) exosomes (FIGS. 25A and 25C) or engineered to display mouse transferrin (mTrf) by differentiated C2C12 myotubes at 5E10-3.13E9 p/mL. FIGS. 25C-25D are representative microscopy images 24 hours post-treatment PTGFRN overexpressing (PrX) exosomes (FIG. 25C) and exosomes engineered to display mouse transferrin (mTrf; FIG. 25D).

FIGS. 26A-26B show the assay (FIG. 26A) and results (FIG. 26B) of functional delivery of ASO using exosomes engineered to display mouse transferrin in C2C12 reporter cells.

### DETAILED DESCRIPTION OF DISCLOSURE

Certain aspects of the present disclosure are directed to extracellular vesicles (EVs) comprising a vNAR that specifically binds transferrin receptor (TfR), e.g., human TfR. In some aspects, the vNAR is loaded on the exterior surface of the EV. In some aspects, the vNAR increases the permeability of the EV across the blood brain barrier in a human subject.

Some aspects, of the disclosure are directed to treating a disease or disorder in a subject in need thereof comprising administering to the subject an EV comprising a vNAR that specifically binds TfR, e.g., human TfR. In some aspects, the disease or disorder comprises a neurological disease or disorder.

### I. Definitions

In order that the present description can be more readily understood, certain terms are first defined. Additional definitions are set forth throughout the detailed description.

It is to be noted that the term “a” or “an” entity refers to one or more of that entity; for example, “a nucleotide sequence,” is understood to represent one or more nucleotide sequences. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Systeme International de Unites (SI) accepted form. Numeric ranges are inclusive of the numbers defining the range. Unless otherwise indicated, nucleotide sequences are written left to right in 5′ to 3′ orientation. Amino acid sequences are written left to right in amino to carboxy orientation. The headings provided herein are not limitations of the various aspects of the disclosure, which can be had by reference to the specification as a whole. Accordingly, the terms defined immediately below are more fully defined by reference to the specification in its entirety.

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower).

As used herein, the term “extracellular vesicle” or “EV” refers to a cell-derived vesicle comprising a membrane that encloses an internal space. Extracellular vesicles comprise all membrane-bound vesicles (e.g., exosomes, nanovesicles) that have a smaller diameter than the cell from which they are derived. In some aspects, extracellular vesicles range in diameter from 20 nm to 1000 nm, and can comprise various macromolecular payload either within the internal space (i.e., lumen), displayed on the external surface of the extracellular vesicle, and/or spanning the membrane. In some aspects, the payload can comprise nucleic acids, proteins, carbohydrates, lipids, small molecules, and/or combinations thereof. In some aspects, an EV comprises one or more payloads or other exogenous biologically active molecules. In some aspects, an EV comprises a targeting moiety that is exogenous to the EV (i.e., not naturally present in the EV) and that allows the EV to target a specific tissue or a specific population of cells. In certain aspects, an extracellular vehicle can further comprise one or more scaffold moieties. By way of example and without limitation, extracellular vesicles include apoptotic bodies, fragments of cells, vesicles derived from cells by direct or indirect manipulation (e.g., by serial extrusion or treatment with alkaline solutions), vesiculated organelles, and vesicles produced by living cells (e.g., by direct plasma membrane budding or fusion of the late endosome with the plasma membrane). Extracellular vesicles can be derived from a living or dead organism, explanted tissues or organs, prokaryotic or eukaryotic cells, and/or cultured cells. In some aspects, the extracellular vesicles are produced by cells that express one or more transgene products. The EVs disclosed herein have been modified and therefore, do not comprise naturally occurring EVs. In some aspects, a composition comprising an EV of the present disclosure comprises a population of exosomes, microvesicles, apoptotic bodies, and/or any combination hereof.

As used herein, the term “exosome” refers to an extracellular vesicle with a diameter between 20-300 nm (e.g., between 40-200 nm). Exosomes comprise a membrane that encloses an internal space (i.e., lumen), and, in some aspects, can be generated from a cell (e.g., producer cell) by direct plasma membrane budding or by fusion of the late endosome with the plasma membrane. In some aspects, an exosome comprises one or more exogenous biologically active molecules (e.g., as described herein). In some aspects, an exosome disclosed herein comprises a targeting moiety that is exogenous to the exosome (i.e., not naturally present in the exosome) and that allows the exosome to target a specific tissue or a specific population of cells. In certain aspects, an exosome further comprises one or more scaffold moieties. As described infra, exosomes can be derived from a producer cell, and isolated from the producer cell based on its size, density, biochemical parameters, or a combination thereof. In some aspects, exosomes of the present disclosure are produced by cells that express one or more transgene products. The exosomes of the present disclosure are modified and therefore, do not comprise naturally occurring exosomes.

As used herein, the term “nanovesicle” refers to an extracellular vesicle with a diameter between 20-250 nm (e.g., between 30-150 nm) and is generated from a cell (e.g., producer cell) by direct or indirect manipulation such that the nanovesicle would not be produced by the cell without the manipulation. Appropriate manipulations of the cell to produce the nanovesicles include but are not limited to serial extrusion, treatment with alkaline solutions, sonication, or combinations thereof. In some aspects, production of nanovesicles can result in the destruction of the producer cell. In some aspects, population of nanovesicles described herein are substantially free of vesicles that are derived from cells by way of direct budding from the plasma membrane or fusion of the late endosome with the plasma membrane. In some aspects, a nanovesicle comprises one or more exogenous biologically active molecules (e.g., disclosed herein). In some aspects, a nanovesicle can further comprise a targeting moiety that is exogenous to the nanovesicle (i.e., not naturally present in the nanovesicle) and that allows the nanovesicle to target a specific tissue or a specific population of cells. In certain aspects, a nanovesicle further comprises one or more scaffold moieties. Nanovesicles, once derived from a producer cell, can be isolated from the producer cell based on its size, density, biochemical parameters, or a combination thereof. As used herein, nanovesicles have been modified and therefore, do not comprise naturally occurring nanovesicles.

As used herein the term “surface-engineered EVs” (e.g., Scaffold X-engineered EVs) refers to an EV with the membrane or the surface modified in its composition, so that the membrane or the surface of the engineered EV, is different from either that of the EV prior to the modification or of the naturally occurring EV. The engineering can be on the surface of the EV or in the membrane of the EV so that the surface of the EV is changed. For example, the membrane is modified in its composition of a protein, a lipid, a small molecule, a carbohydrate, etc. The composition can be changed by a chemical, a physical, or a biological method or by being produced from a cell previously or concurrently modified by a chemical, a physical, or a biological method. Specifically, the composition can be changed by a genetic engineering or by being produced from a cell previously modified by genetic engineering. In some aspects, a surface-engineered EV comprises one or more exogenous biologically active molecules. In certain aspects, the exogenous biologically active molecules can comprise an exogenous protein (i.e., a protein that the EV does not naturally comprise) or a fragment or variant thereof that can be exposed to the surface of the EV or can be an anchoring point (attachment) for a moiety exposed on the surface of the EV. In other aspects, a surface-engineered EV comprises a higher density (e.g., higher number) of a natural exosome protein (e.g., Scaffold X) or a fragment or variant thereof that can be exposed to the surface of the EV or can be an anchoring point (attachment) for a moiety exposed on the surface of the EV.

As used herein the term “lumen-engineered exosome” (e.g., Scaffold Y-engineered exosome) refers to an EV with the membrane or the lumen of the EV modified in its composition so that the lumen of the engineered EV is different from that of the EV prior to the modification or of the naturally occurring EV. The engineering can be directly in the lumen or in the membrane of the EV so that the lumen of the EV is changed. For example, the membrane is modified in its composition of a protein, a lipid, a small molecule, a carbohydrate, etc. so that the lumen of the EV is modified. The composition can be changed by a chemical, a physical, or a biological method or by being produced from a cell previously modified by a chemical, a physical, or a biological method. Specifically, the composition can be changed by a genetic engineering or by being produced from a cell previously modified by genetic engineering. In some aspects, a lumen-engineered exosome comprises one or more exogenous biologically active molecules. In certain aspects, the exogenous biologically active molecules can comprise an exogenous protein (i.e., a protein that the EV does not naturally comprise) or a fragment or variant thereof that can be exposed in the lumen of the EV or can be an anchoring point (attachment) for a moiety exposed on the inner layer of the EV. In other aspects, a lumen-engineered EV comprises a higher density of a natural exosome protein (e.g., Scaffold X or Scaffold Y) or a fragment or variant thereof that can be exposed to the lumen of the exosome or can be an anchoring point (attachment) for a moiety exposed in the lumen of the exosome.

The term “modified,” when used in the context of EVss described herein, refers to an alteration or engineering of an EV and/or its producer cell, such that the modified EV is different from a naturally-occurring EV. In some aspects, a modified EV described herein comprises a membrane that differs in composition of a protein, a lipid, a small molecular, a carbohydrate, etc. compared to the membrane of a naturally-occurring EV (e.g., membrane comprises higher density or number of natural exosome proteins and/or membrane comprises multiple (e.g., at least two) biologically active molecules that are not naturally found in exosomes (e.g., therapeutic molecules (e.g., antigen), targeting moiety, adjuvant, and/or immune modulator). As used herein, biologically active molecules that are not naturally found in exosomes are also described as “exogenous biologically active molecules.” In certain aspects, such modifications to the membrane changes the exterior surface of the EV (e.g., surface-engineered EVss described herein). In certain aspects, such modifications to the membrane changes the lumen of the EV (e.g., lumen-engineered EVss described herein).

As used herein, the terms “binding moiety,” “bio-distribution modifying agent,” and “targeting moiety” are interchangeable and refer to an agent that can modify the distribution of extracellular vesicles (e.g., exosomes, nanovesicles) in vivo or in vitro (e.g., in a mixed culture of cells of different varieties). The targeting moiety can be a biological molecule, such as a protein, a peptide, a lipid, or a synthetic molecule. For example, the targeting moiety can be an antibody (e.g., anti-CD22 nanobody), a synthetic polymer (e.g., PEG), a natural ligand (e.g., CD40L, albumin), a recombinant protein (e.g., XTEN), but not limited thereto. Without being bound to any particular theory, a targeting moiety disclosed herein can modify the distribution of an EV by binding to a marker (also referred to herein as a “target molecule”) expressed on a specific cell type (e.g., a CNS cell, an eye cell, a muscle cell, a macrophage, a cancer cell, or any cell specific to a certain tissue). In some aspects, a targeting moiety disclosed herein binds to a marker for a specific population of immune cells (e.g., CD4+ T cells and/or CD8+ T cells). In certain aspects, the marker is expressed only on CD4+ T cells and/or CD8+ T cells. In some aspects, a marker comprises a CD3 molecule. Accordingly, in certain aspects, a targeting moiety that can be used to increase the distribution of EVs to CD3-expressing immune cells (e.g., CD4+ T cells and/or CD8+ T cells) comprises an anti-CD3 antibody. In certain aspects, the targeting moiety is displayed on the surface of EVs. In some aspects, the targeting moiety can be displayed on the EV surface by being fused to a scaffold protein (e.g., Scaffold X) (e.g., as a genetically encoded fusion molecule). In other aspects, the targeting moiety can be displayed on the EV surface by chemical reaction attaching the targeting moiety to an EV surface molecule. A non-limiting example is PEGylation. In some aspects, a targeting moiety disclosed herein can be combined with a functional moiety, such as a small molecule (e.g., STING, ASO), a drug, and/or a therapeutic protein (e.g., anti-mesothelin antibody/pro-apoptotic proteins).

As used herein, the term “CD47” or “Leukocyte surface antigen CD47” refers to a cell surface antigen that may inhibit uptake of a cell by a macrophage. CD47 is sometimes referred to as the “don't eat me” antigen, as it is a marker of self that may play a role in preventing premature elimination of red blood cells. Unless indicated otherwise, CD47, as used herein, can refer to CD47 from one or more species (e.g., humans (UniProtKB—Q08722), non-human primates, dogs, cats, guinea pigs, rabbits, rats, mice, horses, cattle, and bears).

As used herein, the term “CD24” or “signal transducer CD24” refers to a cell surface antigen that plays a role in the control of autoimmunity. CD24 is believed to modulate B-cell activation responses, promote AG-dependent proliferation of B-cells, and prevent B-cell terminal differentiation into antibody-forming cells. In association with SIGLEC10, CD24 may be involved in the selective suppression of the immune response to danger-associated molecular patterns (DAMPs) such as HMGB1, HSP70 and HSP90. Unless indicated otherwise, CD24, as used herein, can refer to CD24 from one or more species (e.g., humans (UniProtKB—P25063), non-human primates, dogs, cats, guinea pigs, rabbits, rats, mice, horses, cattle, and bears).

As used herein, the term “scaffold moiety” refers to a molecule that can be used to anchor a payload or any other exogenous biologically active molecule of interest (e.g., targeting moiety, adjuvant, and/or immune modulator) to the EV either on the luminal surface or on the exterior surface of the EV. In certain aspects, a scaffold moiety comprises a synthetic molecule. In some aspects, a scaffold moiety comprises a non-polypeptide moiety. In other aspects, a scaffold moiety comprises a lipid, carbohydrate, or protein that naturally exists in the EV. In some aspects, a scaffold moiety comprises a lipid, carbohydrate, or protein that does not naturally exist in the EV. In certain aspects, a scaffold moiety is Scaffold X. In some aspects, a scaffold moiety is Scaffold Y. In further aspects, a scaffold moiety comprises both Scaffold X and Scaffold Y.

As used herein, the term “Scaffold X” refers to exosome proteins that have recently been identified on the surface of exosomes. See, e.g., U.S. Pat. No. 10,195,290, which is incorporated herein by reference in its entirety. Non-limiting examples of Scaffold X proteins include: prostaglandin F2 receptor negative regulator (“the PTGFRN protein”); basigin (“the BSG protein”); immunoglobulin superfamily member 2 (“the IGSF2 protein”); immunoglobulin superfamily member 3 (“the IGSF3 protein”); immunoglobulin superfamily member 8 (“the IGSF8 protein”); integrin beta-1 (“the ITGB1 protein); integrin alpha-4 (“the ITGA4 protein”); 4F2 cell-surface antigen heavy chain (“the SLC3A2 protein”); and a class of ATP transporter proteins (“the ATP1A1 protein,” “the ATP1A2 protein,” “the ATP1A3 protein,” “the ATP1A4 protein,” “the ATP1B3 protein,” “the ATP2B1 protein,” “the ATP2B2 protein,” “the ATP2B3 protein,” “the ATP2B protein”). In some aspects, a Scaffold X protein can be a whole protein or a fragment thereof (e.g., functional fragment, e.g., the smallest fragment that is capable of anchoring another moiety on the exterior surface or on the luminal surface of the EV). In some aspects, a Scaffold X can anchor an exogenous protein (e.g., those disclosed herein, e.g., targeting moiety, therapeutic molecule, adjuvant, and/or immune modulator) to the external surface or the luminal surface of the exosome. Non-limiting examples of other scaffold moieties that can be used with the present disclosure include: aminopeptidase N (CD13); Neprilysin, AKA membrane metalloendopeptidase (MME); ectonucleotide pyrophosphatase/phosphodiesterase family member 1 (ENPP1); Neuropilin-1 (NRP1); CD9, CD63, CD81, PDGFR, GPI anchor proteins, lactadherin, LAMP2, and LAMP2B.

As used herein, the term “Scaffold Y” refers to exosome proteins that were newly identified within the lumen of exosomes. See, e.g., International Publication No. WO/2019/099942, which is incorporated herein by reference in its entirety. Non-limiting examples of Scaffold Y proteins include: myristoylated alanine rich Protein Kinase C substrate (“the MARCKS protein”); myristoylated alanine rich Protein Kinase C substrate like 1 (“the MARCKSL1 protein”); and brain acid soluble protein 1 (“the BASP1 protein”). In some aspects, a Scaffold Y protein can be a whole protein or a fragment thereof (e.g., functional fragment, e.g., the smallest fragment that is capable of anchoring a moiety to the luminal surface of the exosome). In some aspects, a Scaffold Y can anchor an exogenous protein (e.g., those disclosed herein, e.g., targeting moiety, therapeutic molecule, adjuvant, and/or immune modulator) to the luminal surface of the EV.

As used herein, the term “fragment” of a protein (e.g., therapeutic protein, Scaffold X, or Scaffold Y) refers to an amino acid sequence of a protein that is shorter than the naturally-occurring sequence, N- and/or C-terminally deleted or any part of the protein deleted in comparison to the naturally occurring protein. As used herein, the term “functional fragment” refers to a protein fragment that retains protein function. Accordingly, in some aspects, a functional fragment of a Scaffold X protein retains the ability to anchor a moiety on the luminal surface or on the exterior surface of the EV. Similarly, in certain aspects, a functional fragment of a Scaffold Y protein retains the ability to anchor a moiety on the luminal surface of the EV. Whether a fragment is a functional fragment can be assessed by any art known methods to determine the protein content of EVss including Western Blots, FACS analysis and fusions of the fragments with autofluorescent proteins like, e.g., GFP. In certain aspects, a functional fragment of a Scaffold X protein retains at least about 50%, at least about 60%, at least about 70%, at least about 80%, at least about 90% or at least about 100% of the ability, e.g., an ability to anchor a moiety, of the naturally occurring Scaffold X protein. In some aspects, a functional fragment of a Scaffold Y protein retains at least about 50%, at least about 60%, at least about 70%, at least about 80%, at least about 90% or at least about 100% of the ability, e.g., an ability to anchor another molecule, of the naturally occurring Scaffold Y protein.

As used herein, the term “variant” of a molecule (e.g., functional molecule, therapeutic molecule, Scaffold X and/or Scaffold Y) refers to a molecule that shares certain structural and functional identities with another molecule upon comparison by a method known in the art. For example, a variant of a protein can include a substitution, insertion, deletion, frameshift or rearrangement in another protein.

In some aspects, a variant of a Scaffold X comprises a variant having at least about 70% identity to the full-length, mature PTGFRN, BSG, IGSF2, IGSF3, IGSF8, ITGB1, ITGA4, SLC3A2, or ATP transporter proteins or a fragment (e.g., functional fragment) of the PTGFRN, BSG, IGSF2, IGSF3, IGSF8, ITGB1, ITGA4, SLC3A2, or ATP transporter proteins. In some aspects, variants or variants of fragments of PTGFRN share at least about 70%, at least about 80%, at least about 85%, at least about 90%, at least about 95%, at least about 96%, at least about 97%, at least about 98%, or at least about 99% sequence identity with PTGFRN according to SEQ ID NO: 1 or with a functional fragment thereof. In some aspects variants or variants of fragments of BSG share at least about 70%, at least about 80%, at least about 85%, at least about 90%, at least about 95%, at least about 96%, at least about 97%, at least about 98%, or at least about 99% sequence identity with BSG according to SEQ ID NO: 9 or with a functional fragment thereof. In some aspects variants or variants of fragments of IGSF2 share at least about 70%, at least about 80%, at least about 85%, at least about 90%, at least about 95%, at least about 96%, at least about 97%, at least about 98%, or at least about 99% sequence identity with IGSF2 according to SEQ ID NO: 34 or with a functional fragment thereof. In some aspects variants or variants of fragments of IGSF3 share at least about 70%, at least about 80%, at least about 85%, at least about 90%, at least about 95%, at least about 96%, at least about 97%, at least about 98%, or at least about 99% sequence identity with IGSF3 according to SEQ ID NO: 20 or with a functional fragment thereof. In some aspects variants or variants of fragments of IGSF8 share at least about 70%, at least about 80%, at least about 85%, at least about 90%, at least about 95%, at least about 96%, at least about 97%, at least about 98%, or at least about 99% sequence identity with IGSF8 according to SEQ ID NO: 14 or with a functional fragment thereof. In some aspects variants or variants of fragments of ITGB1 share at least about 70%, at least about 80%, at least about 85%, at least about 90%, at least about 95%, at least about 96%, at least about 97%, at least about 98%, or at least about 99% sequence identity with ITGB1 according to SEQ ID NO: 21 or with a functional fragment thereof. In some aspects variants or variants of fragments of ITGA4 share at least about 70%, at least about 80%, at least about 85%, at least about 90%, at least about 95%, at least about 96%, at least about 97%, at least about 98%, or at least about 99% sequence identity with ITGA4 according to SEQ ID NO: 22 or with a functional fragment thereof. In some aspects variants or variants of fragments of SLC3A2 share at least about 70%, at least about 80%, at least about 85%, at least about 90%, at least about 95%, at least about 96%, at least about 97%, at least about 98%, or at least about 99% sequence identity with SLC3A2 according to SEQ ID NO: 23 or with a functional fragment thereof. In some aspects variants or variants of fragments of ATP1A1 share at least about 70%, at least about 80%, at least about 85%, at least about 90%, at least about 95%, at least about 96%, at least about 97%, at least about 98%, or at least about 99% sequence identity with ATP1A1 according to SEQ ID NO: 24 or with a functional fragment thereof. In some aspects variants or variants of fragments of ATP1A2 share at least about 70%, at least about 80%, at least about 85%, at least about 90%, at least about 95%, at least about 96%, at least about 97%, at least about 98%, or at least about 99% sequence identity with ATP1A2 according to SEQ ID NO: 25 or with a functional fragment thereof. In some aspects variants or variants of fragments of ATP1A3 share at least about 70%, at least about 80%, at least about 85%, at least about 90%, at least about 95%, at least about 96%, at least about 97%, at least about 98%, or at least about 99% sequence identity with ATP1A3 according to SEQ ID NO: 26 or with a functional fragment thereof. In some aspects variants or variants of fragments of ATP1A4 share at least about 70%, at least about 80%, at least about 85%, at least about 90%, at least about 95%, at least about 96%, at least about 97%, at least about 98%, or at least about 99% sequence identity with ATP1A4 according to SEQ ID NO: 27 or with a functional fragment thereof. In some aspects variants or variants of fragments of ATP1B3 share at least about 70%, at least about 80%, at least about 85%, at least about 90%, at least about 95%, at least about 96%, at least about 97%, at least about 98%, or at least about 99% sequence identity with ATP1B3 according to SEQ ID NO: 28 or with a functional fragment thereof. In some aspects variants or variants of fragments of ATP2B1 share at least about 70%, at least about 80%, at least about 85%, at least about 90%, at least about 95%, at least about 96%, at least about 97%, at least about 98%, or at least about 99% sequence identity with ATP2B1 according to SEQ ID NO: 29 or with a functional fragment thereof. In some aspects variants or variants of fragments of ATP2B2 share at least about 70%, at least about 80%, at least about 85%, at least about 90%, at least about 95%, at least about 96%, at least about 97%, at least about 98%, or at least about 99% sequence identity with ATP2B2 according to SEQ ID NO: 30 or with a functional fragment thereof. In some aspects variants or variants of fragments of ATP2B3 share at least about 70%, at least about 80%, at least about 85%, at least about 90%, at least about 95%, at least about 96%, at least about 97%, at least about 98%, or at least about 99% sequence identity with ATP2B3 according to SEQ ID NO: 31 or with a functional fragment thereof. In some aspects variants or variants of fragments of ATP2B4 share at least about 70%, at least about 80%, at least about 85%, at least about 90%, at least about 95%, at least about 96%, at least about 97%, at least about 98%, or at least about 99% sequence identity with ATP2B4 according to SEQ ID NO: 32 or with a functional fragment thereof. In some aspects, the variant or variant of a fragment of Scaffold X protein disclosed herein retains the ability to be specifically targeted to EVs. In some aspects, the Scaffold X includes one or more mutations, for example, conservative amino acid substitutions.

In some aspects, a variant of a Scaffold Y comprises a variant having at least 70% identity to MARCKS, MARCKSL1, BASP1 or a fragment of MARCKS, MARCKSL1, or BASP1. In some aspects variants or variants of fragments of MARCKS share at least about 70%, at least about 80%, at least about 85%, at least about 90%, at least about 95%, at least about 96%, at least about 97%, at least about 98%, or at least about 99% sequence identity with MARCKS according to SEQ ID NO: 47 or with a functional fragment thereof. In some aspects variants or variants of fragments of MARCKSL1 share at least about 70%, at least about 80%, at least about 85%, at least about 90%, at least about 95%, at least about 96%, at least about 97%, at least about 98%, or at least about 99% sequence identity with MARCKSL1 according to SEQ ID NO: 48 or with a functional fragment thereof. In some aspects variants or variants of fragments of BASP1 share at least about 70%, at least about 80%, at least about 85%, at least about 90%, at least about 95%, at least about 96%, at least about 97%, at least about 98%, or at least about 99% sequence identity with BASP1 according to SEQ ID NO: 49 or with a functional fragment thereof. In some aspects, the variant or variant of a fragment of Scaffold Y protein retains the ability to be specifically targeted to the luminal surface of EVs. In some aspects, the Scaffold Y includes one or more mutations, e.g., conservative amino acid substitutions.

A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, if an amino acid in a polypeptide is replaced with another amino acid from the same side chain family, the substitution is considered to be conservative. In another aspect, a string of amino acids can be conservatively replaced with a structurally similar string that differs in order and/or composition of side chain family members.

The term “percent sequence identity” or “percent identity” between two polynucleotide or polypeptide sequences refers to the number of identical matched positions shared by the sequences over a comparison window, taking into account additions or deletions (i.e., gaps) that must be introduced for optimal alignment of the two sequences. A matched position is any position where an identical nucleotide or amino acid is presented in both the target and reference sequence. Gaps presented in the target sequence are not counted since gaps are not nucleotides or amino acids. Likewise, gaps presented in the reference sequence are not counted since target sequence nucleotides or amino acids are counted, not nucleotides or amino acids from the reference sequence.

The percentage of sequence identity is calculated by determining the number of positions at which the identical amino-acid residue or nucleic acid base occurs in both sequences to yield the number of matched positions, dividing the number of matched positions by the total number of positions in the window of comparison and multiplying the result by 100 to yield the percentage of sequence identity. The comparison of sequences and determination of percent sequence identity between two sequences can be accomplished using readily available software both for online use and for download. Suitable software programs are available from various sources, and for alignment of both protein and nucleotide sequences. One suitable program to determine percent sequence identity is bl2seq, part of the BLAST suite of programs available from the U.S. government's National Center for Biotechnology Information BLAST web site (blast.ncbi.nlm.nih.gov). Bl2seq performs a comparison between two sequences using either the BLASTN or BLASTP algorithm. BLASTN is used to compare nucleic acid sequences, while BLASTP is used to compare amino acid sequences. Other suitable programs are, e.g., Needle, Stretcher, Water, or Matcher, part of the EMBOSS suite of bioinformatics programs and also available from the European Bioinformatics Institute (EBI) at www.ebi.ac.uk/Tools/psa.

Different regions within a single polynucleotide or polypeptide target sequence that aligns with a polynucleotide or polypeptide reference sequence can each have their own percent sequence identity. It is noted that the percent sequence identity value is rounded to the nearest tenth. For example, 80.11, 80.12, 80.13, and 80.14 are rounded down to 80.1, while 80.15, 80.16, 80.17, 80.18, and 80.19 are rounded up to 80.2. It also is noted that the length value will always be an integer.

One skilled in the art will appreciate that the generation of a sequence alignment for the calculation of a percent sequence identity is not limited to binary sequence-sequence comparisons exclusively driven by primary sequence data. Sequence alignments can be derived from multiple sequence alignments. One suitable program to generate multiple sequence alignments is ClustalW2, available from www.clustal.org. Another suitable program is MUSCLE, available from www.drive5.com/muscle/. ClustalW2 and MUSCLE are alternatively available, e.g., from the EBI.

It will also be appreciated that sequence alignments can be generated by integrating sequence data with data from heterogeneous sources such as structural data (e.g., crystallographic protein structures), functional data (e.g., location of mutations), or phylogenetic data. A suitable program that integrates heterogeneous data to generate a multiple sequence alignment is T-Coffee, available at www.tcoffee.org, and alternatively available, e.g., from the EBI. It will also be appreciated that the final alignment used to calculate percent sequence identity can be curated either automatically or manually.

The polynucleotide variants can contain alterations in the coding regions, non-coding regions, or both. In one aspect, the polynucleotide variants contain alterations which produce silent substitutions, additions, or deletions, but do not alter the properties or activities of the encoded polypeptide. In another aspect, nucleotide variants are produced by silent substitutions due to the degeneracy of the genetic code. In other aspects, variants in which 5-10, 1-5, or 1-2 amino acids are substituted, deleted, or added in any combination. Polynucleotide variants can be produced for a variety of reasons, e.g., to optimize codon expression for a particular host (change codons in the human mRNA to others, e.g., a bacterial host such as E. coli).

Naturally occurring variants are called “allelic variants,” and refer to one of several alternate forms of a gene occupying a given locus on a chromosome of an organism (Genes II, Lewin, B., ed., John Wiley & Sons, New York (1985)). These allelic variants can vary at either the polynucleotide and/or polypeptide level and are included in the present disclosure. Alternatively, non-naturally occurring variants can be produced by mutagenesis techniques or by direct synthesis.

Using known methods of protein engineering and recombinant DNA technology, variants can be generated to improve or alter the characteristics of the polypeptides. For instance, one or more amino acids can be deleted from the N-terminus or C-terminus of the secreted protein without substantial loss of biological function. Ron et al., J. Biol. Chem. 268: 2984-2988 (1993), incorporated herein by reference in its entirety, reported variant KGF proteins having heparin binding activity even after deleting 3, 8, or 27 amino-terminal amino acid residues. Similarly, interferon gamma exhibited up to ten times higher activity after deleting 8-10 amino acid residues from the carboxy terminus of this protein. (Dobeli et al., J Biotechnology 7:199-216 (1988), incorporated herein by reference in its entirety.)

Moreover, ample evidence demonstrates that variants often retain a biological activity similar to that of the naturally occurring protein. For example, Gayle and coworkers (J. Biol. Chem 268:22105-22111 (1993), incorporated herein by reference in its entirety) conducted extensive mutational analysis of human cytokine IL-1a. They used random mutagenesis to generate over 3,500 individual IL-1a mutants that averaged 2.5 amino acid changes per variant over the entire length of the molecule. Multiple mutations were examined at every possible amino acid position. The investigators found that “[m]ost of the molecule could be altered with little effect on either [binding or biological activity].” (See Abstract.) In fact, only 23 unique amino acid sequences, out of more than 3,500 nucleotide sequences examined, produced a protein that significantly differed in activity from wild-type.

As stated above, polypeptide variants include, e.g., modified polypeptides. Modifications include, e.g., acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation (Mei et al., Blood 116:270-79 (2010), which is incorporated herein by reference in its entirety), proteolytic processing, phosphorylation, prenylation, racemization, selenoylation, sulfation, transfer-RNA mediated addition of amino acids to proteins such as arginylation, and ubiquitination. In some aspects, Scaffold X and/or Scaffold Y is modified at any convenient location.

As used herein the terms “linked to,” “conjugated to,” and “anchored to” are used interchangeably and refer to a covalent or non-covalent bond formed between a first moiety and a second moiety, e.g., Scaffold X and a targeting moiety disclosed herein.

The term “encapsulated”, or grammatically different forms of the term (e.g., encapsulation, or encapsulating), refers to a status or process of having a first moiety (e.g., exogenous biologically active molecule, e.g., therapeutic molecule, adjuvant, or immune modulator) inside a second moiety (e.g., an EV) without chemically or physically linking the two moieties. In some aspects, the term “encapsulated” can be used interchangeably with “in the lumen of”. Non-limiting examples of encapsulating a first moiety (e.g., exogenous biologically active molecule, e.g., therapeutic molecule, adjuvant, or immune modulator) into a second moiety (e.g., EVss) are disclosed elsewhere herein.

As used herein, the term “producer cell” refers to a cell used for generating an EV. A producer cell can be a cell cultured in vitro, or a cell in vivo. A producer cell includes, but not limited to, a cell known to be effective in generating EVs e.g., HEK293 cells, Chinese hamster ovary (CHO) cells, mesenchymal stem cells (MSCs), BJ human foreskin fibroblast cells, fHDF fibroblast cells, AGE.HN® neuronal precursor cells, CAP® amniocyte cells, adipose mesenchymal stem cells, RPTEC/TERT1 cells. In certain aspects, a producer cell is not an antigen-presenting cell. In some aspects, a producer cell is not a dendritic cell, a B cell, a mast cell, a macrophage, a neutrophil, Kupffer-Browicz cell, cell derived from any of these cells, or any combination thereof. In some aspects, the EVss useful in the present disclosure do not carry an antigen on MHC class I or class II molecule exposed on the surface of the EV but instead can carry an antigen in the lumen of the EV or on the surface of the EV by attachment to Scaffold X and/or Scaffold Y.

As used herein, the terms “isolate,” “isolated,” and “isolating” or “purify,” “purified,” and “purifying” as well as “extracted” and “extracting” are used interchangeably and refer to the state of a preparation (e.g., a plurality of known or unknown amount and/or concentration) of desired EVs, that have undergone one or more processes of purification, e.g., a selection or an enrichment of the desired EV preparation. In some aspects, isolating or purifying as used herein is the process of removing, partially removing (e.g., a fraction) of the EVs from a sample containing producer cells. In some aspects, an isolated EV composition has no detectable undesired activity or, alternatively, the level or amount of the undesired activity is at or below an acceptable level or amount. In other aspects, an isolated EV composition has an amount and/or concentration of desired EVs at or above an acceptable amount and/or concentration. In other aspects, the isolated EV composition is enriched as compared to the starting material (e.g., producer cell preparations) from which the composition is obtained. This enrichment can be by 10%, 20%, 30%, 40%, 50%, 60%, 70%, 80%, 90%, 95%, 96%, 97%, 98%, 99%, 99.9%, 99.99%, 99.999%, 99.9999%, or greater than 99.9999% as compared to the starting material. In some aspects, isolated EV preparations are substantially free of residual biological products. In some aspects, the isolated EV preparations are 100% free, 99% free, 98% free, 97% free, 96% free, 95% free, 94% free, 93% free, 92% free, 91% free, or 90% free of any contaminating biological matter. Residual biological products can include abiotic materials (including chemicals) or unwanted nucleic acids, proteins, lipids, or metabolites. Substantially free of residual biological products can also mean that the EV composition contains no detectable producer cells and that only EVs are detectable.

As used herein, the term “immune modulator” refers to an agent that acts on a target (e.g., a target cell) that is contacted with the extracellular vesicle, and regulates the immune system. Non-limiting examples of immune modulator that can be introduced into an EV and/or a producer cell include agents such as, modulators of checkpoint inhibitors, ligands of checkpoint inhibitors, cytokines, derivatives thereof, or any combination thereof. The immune modulator can also include an agonist, an antagonist, an antibody, an antigen-binding fragment, a polynucleotide, such as siRNA, miRNA, lncRNA, mRNA, DNA, or a small molecule.

As used herein, the term “payload” refers to an agent that acts on a target (e.g., a target cell) that is contacted with the EV. Non-limiting examples of payload that can be included on the EV are a therapeutic molecule (e.g., protein, enzyme, antigen, immunosuppressive agent, nuceleic acid, or any other therapeutic molecule disclosed herein), an adjuvant, and/or an immune modulator. Payloads that can be introduced into an EV and/or a producer cell include agents such as, nucleotides (e.g., nucleotides comprising a detectable moiety or a toxin or that disrupt transcription), nucleic acids (e.g., DNA or mRNA molecules that encode a polypeptide such as an enzyme, or RNA molecules that have regulatory function such as miRNA, dsDNA, lncRNA, shRNA, siRNA, antisense oligonucleotide, a phosphorodiamidate morpholino oligomer (PMO), or a peptide-conjugated phosphorodiamidate morpholino oligomer (PPMO))), amino acids (e.g., amino acids comprising a detectable moiety or a toxin or that disrupt translation), polypeptides (e.g., therapeutic protein, enzymes), lipids, carbohydrates, and small molecules (e.g., small molecule drugs and toxins). In certain aspects, a payload comprises an exogenous biologically active molecule (e.g., those disclosed herein).

As used herein, the term “loaded,” “loading,” and “load” refer to the localization of a target molecule, e.g, a single-domain antigen-binding moeity, a payload, or a biologically active molecule disclosed herein, into an EV. A target molecule can be loaded into the lumen of the EV, onto the luminal surface of the EV, or on the external surface of the EV, using any methods known in the art. In some aspects, the target molecule is loaded by expressing the target molecule in an EV-producing cell. In some aspects, the target molecule is loaded by expressing the target molecule in an EV-producing cell as a fusion construct with a protein that localizes to an EV, e.g., a scaffold moiety disclosed herein. In some aspects, the target molecule is loaded to the surface (luminal and/or external) of the EV by chemically linking the target molecule to the surface (luminal and/or external) of the EV.

As used herein, the term “biologically active molecule” refers to an agent that has activity in a biological system (e.g., a cell or a human subject), including, but not limited to a protein, polypeptide or peptide including, but not limited to, a structural protein, an enzyme, a cytokine (such as an interferon and/or an interleukin) an antibiotic, a polyclonal or monoclonal antibody, or an effective part thereof, such as an Fv fragment, which antibody or part thereof can be natural, synthetic or humanized, a peptide hormone, a receptor, a signaling molecule or other protein; a nucleic acid, as defined below, including, but not limited to, an oligonucleotide or modified oligonucleotide, an antisense oligonucleotide or modified antisense oligonucleotide, cDNA, genomic DNA, an artificial or natural chromosome (e.g. a yeast artificial chromosome) or a part thereof, RNA, including mRNA, tRNA, rRNA or a ribozyme, or a peptide nucleic acid (PNA); a virus or virus-like particles; a nucleotide or ribonucleotide or synthetic analogue thereof, which can be modified or unmodified; an amino acid or analogue thereof, which can be modified or unmodified; a non-peptide (e.g., steroid) hormone; a proteoglycan; a lipid; or a carbohydrate. In certain aspects, a biologically active molecule comprises a therapeutic molecule (e.g., protein, enzyme, antigen, or other therapeutic molecule disclosed herein), a targeting moiety (e.g., an antibody or an antigen-binding fragment thereof), an adjuvant, an immune modulator, or any combination thereof. In some aspects, the biologically active molecule comprises a macromolecule (e.g., a protein, an antibody, an enzyme, a peptide, DNA, RNA, or any combination thereof). In some aspects, the biologically active molecule comprises a small molecule (e.g., an antisense oligomer (ASO), PMO, morpholino, siRNA, miRNA, antagomir, an siRNA, STING, a pharmaceutical drug, or any combination thereof). In some aspects, the biologically active molecules are exogenous to the exosome, i.e., not naturally found in the exosome.

As used herein, the term “therapeutic molecule” refers to any molecule that can treat and/or prevent a disease or disorder in a subject (e.g., human subject). Examples of the therapeutic molecules are shown elsewhere herein.

As used herein, the term “antibody” encompasses an immunoglobulin whether natural or partly or wholly synthetically produced, and fragments thereof. The term also covers any protein having a binding domain that is homologous to an immunoglobulin binding domain. “Antibody” further includes a polypeptide comprising a framework region from an immunoglobulin gene or fragments thereof that specifically binds and recognizes an antigen. Use of the term antibody is meant to include whole antibodies, polyclonal, monoclonal and recombinant antibodies, fragments thereof, and further includes single-chain antibodies, humanized antibodies, murine antibodies, chimeric, mouse-human, mouse-primate, primate-human monoclonal antibodies, anti-idiotype antibodies, antibody fragments, such as, e.g., scFv, (scFv)₂, Fab, Fab′, and F(ab′)₂, F(ab1)₂, Fv, dAb, and Fd fragments, diabodies, and antibody-related polypeptides. Antibody includes bispecific antibodies and multispecific antibodies so long as they exhibit the desired biological activity or function. In some aspects, the antibody or antigen-binding fragment thereof comprises a scFv, scFab, scFab-Fc, nanobody, or any combination thereof. In some aspects, the antibody or antigen-binding fragment thereof comprises an agonist antibody, a blocking antibody, a targeting antibody, a fragment thereof, or a combination thereof. In some aspects, the agonist antibody is a CD40L agonist. In some aspects, the blocking antibody binds a target protein selected from programmed death 1 (PD-1), programmed death ligand 1 (PD-L1), cytotoxic T-lymphocyte-associated protein 4, and any combination thereof.

As used herein, a “vNAR” refers to a polypeptide comprising a variable region of an IgNAR antibody or an antigen-binding fragment thereof. IgNAR antibodies are heavy-chian homodimers made up of two heavy chains, each heavy chain having a variable heavy region (vNAR) and five constant heavy regions. IgNAR are naturally expressed in cartilaginous fish and sharks (see, e.g., Dooley et al., Molecular Immunology 40:25-33 (2003); and Int'l Publ. No. WO 2016/077840 A2; each of which is incorporated by reference herein in its entirety). In some aspects, the vNAR comprises the general structure (FW1-CDR1-FW2-3-CDR3-FW4). See, e.g., U.S. 2017-0348416, which is incorporated by reference herein in its entirety. vNAR attributes include high affinity for target, ease of expression, stability, solubility, multi-specificity, and increased potential for solid tissue penetration. See Ubah et al. Biochem. Soc. Trans. (2018) 46(6):1559-1565.

Camelid antibodies are characterized as homodimers made up of two heavy chains, each heavy chain having a single variable heavy region (a VHH) and two constant heavy regions. As such, camelid antibodies, or fragments thereof, can be used in the subject application in the place of the vNAR. In certain aspects, the EV comprises a VHH that specifically binds TfR. As used herein, a “VHH” (also referred to as a nanobody) is a single variable domain on a heavy chain antibody, which lacks a constant region. In nature, a VHH is the antigen-binding portion of a heavy chain only antibody (HcAb), such as antibodies produced naturally in sharks and camelids (see, e.g., Bever at al., Anal. Bioananl Chem 408(22):5985-6002 (2016), which is incorporated by reference herein in its entirety).

The terms “individual,” “subject,” “host,” and “patient,” are used interchangeably herein and refer to any mammalian subject for whom diagnosis, treatment, or therapy is desired, particularly humans. The compositions and methods described herein are applicable to both human therapy and veterinary applications. In some aspects, the subject is a mammal, and in other aspects the subject is a human. As used herein, a “mammalian subject” includes all mammals, including without limitation, humans, domestic animals (e.g., dogs, cats and the like), farm animals (e.g., cows, sheep, pigs, horses and the like) and laboratory animals (e.g., monkey, rats, mice, rabbits, guinea pigs and the like).

As used herein, the term “substantially free” means that the sample comprising EVs comprise less than 10% of macromolecules by mass/volume (m/v) percentage concentration. Some fractions can contain less than 0.001%, less than 0.01%, less than 0.05%, less than 0.1%, less than 0.2%, less than 0.3%, less than 0.4%, less than 0.5%, less than 0.6%, less than 0.7%, less than 0.8%, less than 0.9%, less than 1%, less than 2%, less than 3%, less than 4%, less than 5%, less than 6%, less than 7%, less than 8%, less than 9%, or less than 10% (m/v) of macromolecules.

As used herein, the term “macromolecule” means nucleic acids, contaminant proteins, lipids, carbohydrates, metabolites, or a combination thereof.

As used herein, the term “conventional exosome protein” means a protein previously known to be enriched in exosomes, including but is not limited to CD9, CD63, CD81, PDGFR, GPI anchor proteins, lactadherin LAMP2, and LAMP2B, a fragment thereof, or a peptide that binds thereto.

“Administering,” as used herein, means to give a composition comprising an EV disclosed herein to a subject via a pharmaceutically acceptable route. Routes of administration can be intravenous, e.g., intravenous injection and intravenous infusion. Additional routes of administration include, e.g., subcutaneous, intramuscular, oral, nasal, and pulmonary administration. EVss can be administered as part of a pharmaceutical composition comprising at least one excipient.

An “immune response,” as used herein, refers to a biological response within a vertebrate against foreign agents or abnormal, e.g., cancerous cells, which response protects the organism against these agents and diseases caused by them. An immune response is mediated by the action of one or more cells of the immune system (for example, a T lymphocyte, B lymphocyte, natural killer (NK) cell, macrophage, eosinophil, mast cell, dendritic cell or neutrophil) and soluble macromolecules produced by any of these cells or the liver (including antibodies, cytokines, and complement) that results in selective targeting, binding to, damage to, destruction of, and/or elimination from the vertebrate's body of invading pathogens, cells or tissues infected with pathogens, cancerous or other abnormal cells, or, in cases of autoimmunity or pathological inflammation, normal human cells or tissues. An immune reaction includes, e.g., activation or inhibition of a T cell, e.g., an effector T cell, a Th cell, a CD4+ cell, a CD8+ T cell, or a Treg cell, or activation or inhibition of any other cell of the immune system, e.g., NK cell. Accordingly an immune response can comprise a humoral immune response (e.g., mediated by B-cells), cellular immune response (e.g., mediated by T cells), or both humoral and cellular immune responses. In some aspects, an immune response is an “inhibitory” immune response. An inhibitory immune response is an immune response that blocks or diminishes the effects of a stimulus (e.g., antigen). In certain aspects, the inhibitory immune response comprises the production of inhibitory antibodies against the stimulus. In some aspects, an immune response is a “stimulatory” immune response. A stimulatory immune response is an immune response that results in the generation of effectors cells (e.g., cytotoxic T lymphocytes) that can destroy and clear a target antigen (e.g., tumor antigen or viruses).

As used herein, the term “immune cells” refers to any cells of the immune system that are involved in mediating an immune response. Non-limiting examples of immune cells include a T lymphocyte, B lymphocyte, natural killer (NK) cell, macrophage, eosinophil, mast cell, dendritic cell, neutrophil, or combination thereof. In some aspects, an immune cell expresses CD3. In certain aspects, the CD3-expressing immune cells are T cells (e.g., CD4+ T cells or CD8+ T cells). In some aspects, an immune cell that can be targeted with a targeting moiety disclosed herein (e.g., anti-CD3) comprises a naïve CD4+ T cell. In some aspects, an immune cell comprises a memory CD4+ T cell. In some aspects, an immune cell comprises an effector CD4+ T cell. In some aspects, an immune cell comprises a naïve CD8+ T cell. In some aspects, an immune cell comprises a memory CD8+ T cell. In some aspects, an immune cell comprises an effector CD8+ T cell.

As used herein, the term “T cell” or “T-cell” refers to a type of lymphocyte that matures in the thymus. T cells play an important role in cell-mediated immunity and are distinguished from other lymphocytes, such as B cells, by the presence of a T-cell receptor on the cell surface. T-cells include all types of immune cells expressing CD3, including T-helper cells (CD4+ cells), cytotoxic T-cells (CD8+ cells), natural killer T-cells, T-regulatory cells (Treg), and gamma-delta T cells.

“Treat,” “treatment,” or “treating,” as used herein, refers to, e.g., the reduction in severity of a disease or condition; the reduction in the duration of a disease course; the amelioration or elimination of one or more symptoms associated with a disease or condition; the provision of beneficial effects to a subject with a disease or condition, without necessarily curing the disease or condition. The term also include prophylaxis or prevention of a disease or condition or its symptoms thereof. In one aspect, the term “treating” or “treatment” means inducing an immune response in a subject against an antigen.

“Prevent” or “preventing,” as used herein, refers to decreasing or reducing the occurrence or severity of a particular outcome. In some aspects, preventing an outcome is achieved through prophylactic treatment.

### II. Compositions of the Disclosure

Certain aspects of the present disclosure are directed to EVs comprising an antigen-binding moeity that specifically binds transferrin receptor (TfR). In some aspects, the antigen-binding moeity comprises one or more single-domain antigen-binding moieties. In some aspects, the one or more single-domain antigen-binding moieties are selected from a VHH, a vNAR, an antigen-binding fragment of a VHH, an antigen-binding fragment of a vNAR, and any combination thereof.

In some aspects, the antigen-binding moeity, e.g., vNAR or VHH, is loaded on the external surface of the EV. In some aspects, the antigen-binding moeity, e.g., vNAR or VHH, specifically binds human TfR. Transferrin receptors, e.g., TfR1 or TfR2, are carrier proteins for transferrin. Transferrin receptors import iron by internalizing the transferrin-ion complex through receptor-mediated endocytosis.

TfR1 (see, e.g., UniProt P02786_TFR1 Human) or transferrin receptor 1 (also known as cluster of differentiation 71 or CD71) is expressed on the endothelial cells of the blood-brain barrier (BBB). TfR1 is known to be expressed in a variety of cells such as red blood cells, monocytes, hepatocytes, intestinal cells, and erythroid cells, and is upregulated in rapidly dividing cells such as tumor cells (non small cell lung cancer, colon cancer, and leukemia) as well as in tissue affected by disorders such as acute respiratory distress syndrome (ARDS). TfR2 is primarily expressed in liver and erythroid cells, is found to a lesser extent in lung, spleen and muscle, and has a 45% identity and 66% similarity with TfR1. TfR1 is a transmembrane receptor that forms a homodimer of 760 residues with disulfide bonds and a molecular weight of 90 kDa. Affinity for transferrin varies between the two receptor types, with the affinity for TfR1 being at least 25-30 fold higher than that of TfR2.

Binding to TfR1 allows the transit of large molecules, e.g., antibodies, into the brain. Some TfR1-targeting antibodies have been shown to cross the blood-brain barrier, without interfering with the uptake of iron. Amongst those are the mouse anti rat-TfR antibody OX26 and the rat anti mouse-TfR antibody 8D3. The affinity of the antibody-TfR interaction is important to determine the success of transcytotic transport over endothelial cells of the BBB. Monovalent TfR interaction favors BBB transport due to altered intracellular sorting pathways. Avidity effects of bivalent interactions redirecting transport to the lysosome. Also, reducing TfR binding affinity directly promotes dissociation from the TfR which increases brain parenchymal exposure of the TfR binding antibody. See, e.g., U.S. Pat. No. 8,821,943, which is herein incorporated by reference in its entirety.

II.A. Antigen-Binding Moeities

Any antigen-binding moeity known in the art that is capable of specifically binding TfR, e.g., human TfR, can be used in the compositions and/or methods disclosed herein. In some aspects, the antigen-binding moeity comprises an antibody or an antigen-binding fragment thereof. In some aspects, the antibody is selected from an IgG, IgA, IgE, IgM, or any combination thereof. In certain aspects, the antibody is an IgG antibody. In some aspects, the antibody is a monoclonal antibody. In some aspects, the antigen-binding moeity is an antigen-binding fragment of an antibody. In some aspects, the antigen-binding moeity comprises an scFv. In some aspects, the antigen-binding moeity comprises a camelid antibody. In some aspects, the antigen-binding moeity comprises an IgNAR. In some aspects, the antigen-binding moeity comprises vNAR.

In certain aspects, the antigen-binding moeity comprises a single-domain antigen-binding moeity or an antigen-binding fragment thereof. Single-domain antigen-binding moieties, e.g., as disclosed herein, are smaller than conventional human antibodies and scFv fragments thereof, allowing for generation of EVs having a higher concentration of surface-loaded antigen-binidng moieties. For example, VHH and vNAR antigen binding domains each have a molecular weight of about 12-15 kDa, which is about one-tenth the size of an IgG (about 150 kDa) and nearly half the size of an scFv (about 27 kDa). Using a single-domain antigen-binding moeity allows for a higher concentration of the antigen-binding moeity to be loaded onto the EV which may enhance the ability of the EV to target and interact with TfR. Accordingly, in some aspects, single-chain antigen-binding moiety of the present disclosure is smaller than an scFv.

In some aspects, the single-domain antigen-binding moeity comprises a VHH. Any VHH known in the art can be used in the methods disclosed herein. In some aspects, the VHH is derived from a camelid antibody. In some aspects, the VHH is a fragment of a camelid antibody. In some aspects, the VHH is a synthetic polypeptide. In some aspects, the VHH is a recombinant polypeptide. In some aspects, the VHH comprises one or more mutations to enhance the binding affinity of the VHH to a target antigen. In some aspects, the VHH comprises one or more mutations to enhance the stability of the VHH.

In some aspects, the single-domain antigen-binding moeity comprises a vNAR. Any vNAR known in the art can be used in the methods disclosed herein. In some aspects, the vNAR is derived from an IgNAR. In some aspects, the vNAR is derived from an IgNAR. In some aspects, the vNAR is a fragment of a shark IgNAR antibody. In some aspects, the vNAR is a synthetic polypeptide. In some aspects, the vNAR is a recombinant polypeptide. In some aspects, the vNAR comprises one or more mutations to enhance the binding affinity of the vNAR to a target antigen. In some aspects, the vNAR comprises one or more mutations to enhance the stability of the vNAR.

In some aspects, the antigen-binding moeity, e.g., vNAR or VHH, specifically binds human TfR with a K_{D} of less than 5×10⁻⁶, 2×10⁻⁶, 1×10⁻⁶, 5×10⁻⁷, 1×10⁻⁷, 5×10⁻⁸, 1×10⁻⁸, 5×10⁻⁹, or 1×10⁻⁹M. In some aspects, the antigen-binding moeity, e.g., vNAR or VHH, specifically binds human TfR with a K_{D} of less than 5×10⁻⁶ M. In some aspects, the antigen-binding moeity, e.g., vNAR or VHH, specifically binds human TfR with a K_{D} of less than 2×10⁻⁶ M. In some aspects, the antigen-binding moeity, e.g., vNAR or VHH, specifically binds human TfR with a K_{D} of less than 1×10⁻⁶ M. In some aspects, the antigen-binding moeity, e.g., vNAR or VHH, specifically binds human TfR with a K_{D} of less than 5×10⁻⁷ M. In some aspects, the antigen-binding moeity, e.g., vNAR or VHH, specifically binds human TfR with a K_{D} of less than 1×10⁻⁷ M. In some aspects, the antigen-binding moeity, e.g., vNAR or VHH, specifically binds human TfR with a K_{D} of less than 5×10⁻⁸ M. In some aspects, the antigen-binding moeity, e.g., vNAR or VHH, specifically binds human TfR with a K_{D} of less than 1×10⁻⁸M. In some aspects, the antigen-binding moeity, e.g., vNAR or VHH, specifically binds human TfR with a K_{D} of less than 5×10⁻⁹ M. In some aspects, the antigen-binding moeity, e.g., vNAR or VHH, specifically binds human TfR with a K_{D} of less than 1×10⁻⁹ M. In some aspects, the antigen-binding moeity, e.g., vNAR or VHH, comprises or consists essentially of a vNAR scaffold with any one CDR1 peptide in Table 1 of U.S. 2017-0348416 in combination with any one CDR3 peptide in Table 1 of U.S. 2017-0348416 (which is incorporated by reference herein in its entirety).

In certain aspects, the vNAR is selected from any vNAR disclosed in US Publication No. 2017/348416, which is incorporated by reference herein in its entirety. In some aspects, the vNAR comprises the amino acid sequence set forth in SEQ ID NO: 193. In some aspects, the vNAR consists of the amino acid sequence set forth in SEQ ID NO: 193. In some aspects, the vNAR comprises the amino acid sequence set forth in SEQ ID NO: 194. In some aspects, the vNAR consists of the amino acid sequence set forth in SEQ ID NO: 194. In some aspects, the vNAR comprises the amino acid sequence set forth in SEQ ID NO: 195. In some aspects, the vNAR consists of the amino acid sequence set forth in SEQ ID NO: 195.

As discussed herein, fusion of an antigen-binding moeity, e.g., vNAR or VHH, that specifically binds TfR to an EV can increase the permeability of the EV across the blood brain barrier through internalization of the TfR upon binding of the antigen-binding moeity, e.g., vNAR or VHH. In some aspects, the antigen-binding moeity, e.g., vNAR or VHH, increases the permeability of the EV across the blood brain barrier by at least about 10%, at least about 20%, at least about 30%, at least about 40%, at least about 75%, at least about 100%, at least about 150%, at least about 200%, at least about 250%, at least about 300%, at least about 350%, at least about 400%, at least about 450%, at least about 500%, at least about 600%, at least about 700%, at least about 800%, at least about 900%, or at least about 1000%, as compared to a reference EV not comprising an antigen-binding moeity, e.g., vNAR or VHH, that specifically binds human TfR. In some aspects, the antigen-binding moeity comprises a single-domain antigen-binding moeity, e.g., a vNAR or a VHH, wherein the single-domain antigen-binding moeity, e.g., a vNAR or a VHH, increases the permeability of the EV across the blood brain barrier by at least about 10%, at least about 20%, at least about 30%, at least about 40%, at least about 75%, at least about 100%, at least about 150%, at least about 200%, at least about 250%, at least about 300%, at least about 350%, at least about 400%, at least about 450%, at least about 500%, at least about 600%, at least about 700%, at least about 800%, at least about 900%, or at least about 1000%, as compared to an EV comprising an IgG antibody or a fragment thereof that specifically binds human TfR. In some aspects, the antigen-binding moeity comprises a single-domain antigen-binding moeity, e.g., a vNAR or a VHH, wherein the single-domain antigen-binding moeity, e.g., a vNAR or a VHH, increases the permeability of the EV across the blood brain barrier by at least about 10%, at least about 20%, at least about 30%, at least about 40%, at least about 75%, at least about 100%, at least about 150%, at least about 200%, at least about 250%, at least about 300%, at least about 350%, at least about 400%, at least about 450%, at least about 500%, at least about 600%, at least about 700%, at least about 800%, at least about 900%, or at least about 1000%, as compared to an EV comprising an scFv that specifically binds human TfR.

In some aspects, the EV comprises the antigen-binding moeity comprises at least two, at least three, at least four, at least five, or at least six single-domain antigen-binding moieties. In certain aspects, the antigen-binding moeity comprises at least two single-domain antigen-binding moieties. In some aspects, the at least two single-domain antigen-binding moieties are the same, e.g., the at least two single-domain antigen-binding moieties bind the same epitope on TfR. In other aspects, the at least two single-domain antigen-binding moieties are different. In certain aspects, the at least two single-domain antigen-binding moieties comprise (i) a first single-domain antigen-binding moiety that binds a first epitope on TfR and (ii) a second single-domain antigen-binding moiety binds a second epitope on TfR.

In some aspects, a first single-domain antigen-binding moiety is linked to a second single-domain antigen-binding moeity by a linker, e.g., any linker disclosed herein. In some aspects, the linker is a peptide linker.

II.B. Extracellular Vesicles

As described herein, extracellular vesicles are lipid-based particles having a diameter between about 20-300 nm. In certain embodiments, an EV of the present disclosure has a diameter between about 20 nm and about 290 nm, between about 20 nm and about 280 nm, between about 20 nm and about 270 nm, between about 20 nm and about 260 nm, 20 nm and about 250 nm, between about 20 nm and about 240 nm, between about 20 nm and about 230 nm, between about 20 nm and about 220 nm, between about 20 nm and about 210 nm, between about 20 nm and about 200 nm, between about 20 nm and about 190 nm, between about 20 nm and about 180 nm, between about 20 nm and about 170 nm, between about 20 nm and about 160 nm, between about 20 nm and about 150 nm, between about 20 nm and about 140 nm, between about 20 nm and about 130 nm, between about 20 nm and about 120 nm, between about 20 nm and about 110 nm, between about 20 nm and about 100 nm, between about 20 nm and about 90 nm, between about 20 nm and about 80 nm, between about 20 nm and about 70 nm, between about 20 nm and about 60 nm, between about 20 nm and about 50 nm, between about 20 nm and about 40 nm, between about 20 nm and about 30 nm, between about 30 nm and about 300 nm, between about 30 nm and about 290 nm, between about 30 nm and about 280 nm, between about 30 nm and about 270 nm, between about 30 nm and about 260 nm, between about 30 nm and about 250 nm, between about 30 nm and about 240 nm, between about 30 nm and about 230 nm, between about 30 nm and about 220 nm, between about 30 nm and about 210 nm, between about 30 nm and about 200 nm, between about 30 nm and about 190 nm, between about 30 nm and about 180 nm, between about 30 nm and about 170 nm, between about 30 nm and about 160 nm, between about 30 nm and about 150 nm, between about 30 nm and about 140 nm, between about 30 nm and about 130 nm, between about 30 nm and about 120 nm, between about 30 nm and about 110 nm, between about 30 nm and about 100 nm, between about 30 nm and about 90 nm, between about 30 nm and about 80 nm, between about 30 nm and about 70 nm, between about 30 nm and about 60 nm, between about 30 nm and about 50 nm, between about 30 nm and about 40 nm, between about 40 nm and about 300 nm, between about 40 nm and about 290 nm, between about 40 nm and about 280 nm, between about 40 nm and about 270 nm, between about 40 nm and about 260 nm, between about 40 nm and about 250 nm, between about 40 nm and about 240 nm, between about 40 nm and about 230 nm, between about 40 nm and about 220 nm, between about 40 nm and about 210 nm, between about 40 nm and about 200 nm, between about 40 nm and about 190 nm, between about 40 nm and about 180 nm, between about 40 nm and about 170 nm, between about 40 nm and about 160 nm, between about 40 nm and about 150 nm, between about 40 nm and about 140 nm, between about 40 nm and about 130 nm, between about 40 nm and about 120 nm, between about 40 nm and about 110 nm, between about 40 nm and about 100 nm, between about 40 nm and about 90 nm, between about 40 nm and about 80 nm, between about 40 nm and about 70 nm, between about 40 nm and about 60 nm, between about 40 nm and about 50 nm, between about 50 nm and about 300 nm, between about 50 nm and about 290 nm, between about 50 nm and about 280 nm, between about 50 nm and about 270 nm, between about 50 nm and about 260 nm, between about 50 nm and about 250 nm, between about 50 nm and about 240 nm, between about 50 nm and about 230 nm, between about 50 nm and about 220 nm, between about 50 nm and about 210 nm, between about 50 nm and about 200 nm, between about 50 nm and about 190 nm, between about 50 nm and about 180 nm, between about 50 nm and about 170 nm, between about 50 nm and about 160 nm, between about 50 nm and about 150 nm, between about 50 nm and about 140 nm, between about 50 nm and about 130 nm, between about 50 nm and about 120 nm, between about 50 nm and about 110 nm, between about 50 nm and about 100 nm, between about 50 nm and about 90 nm, between about 50 nm and about 80 nm, between about 50 nm and about 70 nm, between about 50 nm and about 60 nm, between about 60 nm and about 300 nm, between about 60 nm and about 290 nm, between about 60 nm and about 280 nm, between about 60 nm and about 270 nm, between about 60 nm and about 260 nm, between about 60 nm and about 250 nm, between about 60 nm and about 240 nm, between about 60 nm and about 230 nm, between about 60 nm and about 220 nm, between about 60 nm and about 210 nm, between about 60 nm and about 200 nm, between about 60 nm and about 190 nm, between about 60 nm and about 180 nm, between about 60 nm and about 170 nm, between about 60 nm and about 160 nm, between about 60 nm and about 150 nm, between about 60 nm and about 140 nm, between about 60 nm and about 130 nm, between about 60 nm and about 120 nm, between about 60 nm and about 110 nm, between about 60 nm and about 100 nm, between about 60 nm and about 90 nm, between about 60 nm and about 80 nm, between about 60 nm and about 70 nm, between about 70 nm and about 300 nm, between about 70 nm and about 290 nm, between about 70 nm and about 280 nm, between about 70 nm and about 270 nm, between about 70 nm and about 260 nm, between about 70 nm and about 250 nm, between about 70 nm and about 240 nm, between about 70 nm and about 230 nm, between about 70 nm and about 220 nm, between about 70 nm and about 210 nm, between about 70 nm and about 200 nm, between about 70 nm and about 190 nm, between about 70 nm and about 180 nm, between about 70 nm and about 170 nm, between about 70 nm and about 160 nm, between about 70 nm and about 150 nm, between about 70 nm and about 140 nm, between about 70 nm and about 130 nm, between about 70 nm and about 120 nm, between about 70 nm and about 110 nm, between about 70 nm and about 100 nm, between about 70 nm and about 90 nm, between about 70 nm and about 80 nm, between about 80 nm and about 300 nm, between about 80 nm and about 290 nm, between about 80 nm and about 280 nm, between about 80 nm and about 270 nm, between about 80 nm and about 260 nm, between about 80 nm and about 250 nm, between about 80 nm and about 240 nm, between about 80 nm and about 230 nm, between about 80 nm and about 220 nm, between about 80 nm and about 210 nm, between about 80 nm and about 200 nm, between about 80 nm and about 190 nm, between about 80 nm and about 180 nm, between about 80 nm and about 170 nm, between about 80 nm and about 160 nm, between about 80 nm and about 150 nm, between about 80 nm and about 140 nm, between about 80 nm and about 130 nm, between about 80 nm and about 120 nm, between about 80 nm and about 110 nm, between about 80 nm and about 100 nm, between about 80 nm and about 90 nm, between about 90 nm and about 300 nm, between about 90 nm and about 290 nm, between about 90 nm and about 280 nm, between about 90 nm and about 270 nm, between about 90 nm and about 260 nm, between about 90 nm and about 250 nm, between about 90 nm and about 240 nm, between about 90 nm and about 230 nm, between about 90 nm and about 220 nm, between about 90 nm and about 210 nm, between about 90 nm and about 200 nm, between about 90 nm and about 190 nm, between about 90 nm and about 180 nm, between about 90 nm and about 170 nm, between about 90 nm and about 160 nm, between about 90 nm and about 150 nm, between about 90 nm and about 140 nm, between about 90 nm and about 130 nm, between about 90 nm and about 120 nm, between about 90 nm and about 110 nm, between about 90 nm and about 100 nm, between about 100 nm and about 300 nm, between about 110 nm and about 290 nm, between about 120 nm and about 280 nm, between about 130 nm and about 270 nm, between about 140 nm and about 260 nm, between about 150 nm and about 250 nm, between about 160 nm and about 240 nm, between about between about 170 nm and about 230 nm, between about 180 nm and about 220 nm, or between about 190 nm and about 210 nm. The size of the EV described herein can be measured according to methods described, infra.

EVs of the present disclosure comprise a membrane (“EV membrane”), comprising an external surface (e.g., an extracellular surface) and an internal surface (e.g., a luminal surface). In certain embodiments, the internal surface faces the inner core (i.e., lumen) of the EV. In certain embodiments, the external surface can be in contact with the endosome, the multivesicular bodies, or the membrane/cytoplasm of a producer cell.

In some embodiments, the EV membrane comprises a bi-lipid membrane, e.g., a lipid bilayer. In some embodiments, the EV membrane comprises lipids and fatty acids. In some embodiments, the EV membrane comprises phospholipids, glycolipids, fatty acids, sphingolipids, phosphoglycerides, sterols, cholesterols, and phosphatidylserines.

In some embodiments, the EV membrane comprises an inner leaflet and an outer leaflet. The composition of the inner and outer leaflet can be determined by transbilayer distribution assays known in the art, see, e.g., Kuypers et al., Biochem Biophys Acta 1985 819:170. In some embodiments, the composition of the outer leaflet is between approximately 70-90% choline phospholipids, between approximately 0-15% acidic phospholipids, and between approximately 5-30% phosphatidylethanolamine. In some embodiments, the composition of the inner leaflet is between approximately 15-40% choline phospholipids, between approximately 10-50% acidic phospholipids, and between approximately 30-60% phosphatidylethanolamine.

In some embodiments, the EV membrane comprises one or more polysaccharides, such as glycan. In some embodiments, the EV comprises one or more multilamellar bodies within the lumen of the EV.

In some aspects, the EV is an exosome, a microvesicle, an apoptotic body, or any combination thereof. In certain aspects, the EV is an exosome. In certain aspects, the EV is a microvesicle. In certain aspects, the EV is an apoptotic body.

In some aspects, the EV is modified to have a high density of vNAR on the external surface of the EV. Without being bound by any particular mechanism, having a higher density of the vNAR on the external surface of the EV is believed to increase the tropism of the EV to TfR in vivo, thereby increasing the efficacy of the EV interacting with TfR and being transported across the blood brain barrier. In some aspects, the vNAR is present at a concentration of at least about 100 copies per EV, at least about 150 copies per EV, at least about 200 copies per EV, at least about 250 copies per EV, at least about 300 copies per EV, at least about 350 copies per EV, at least about 400 copies per EV, at least about 450 copies per EV, at least about 500 copies per EV, at least about 600 copies per EV, at least about 700 copies per EV, at least about 800 copies per EV, at least about 900 copies per EV, at least about 1000 copies per EV, at least about 1250 copies per EV, at least about 1500 copies per EV, at least about 2000 copies per EV, at least about 2500 copies per EV, at least about 3000 copies per EV, at least about 3500 copies per EV, at least about 4000 copies per EV, at least about 4500 copies per EV, or at least about 5000 copies per EV. In some aspects, the vNAR is present at a concentration of at least about 500 copies per EV. In some aspects, the vNAR is present at a concentration of at least about 750 copies per EV. In some aspects, the vNAR is present at a concentration of at least about 1000 copies per EV. In some aspects, the vNAR is present at a concentration of at least about 1250 copies per EV. In some aspects, the vNAR is present at a concentration of at least about 1500 copies per EV. In some aspects, the vNAR is present at a concentration of at least about 1750 copies per EV. In some aspects, the vNAR is present at a concentration of at least about 2000 copies per EV. In some aspects, the vNAR is present at a concentration of at least about 2250 copies per EV. In some aspects, the vNAR is present at a concentration of at least about 2500 copies per EV. In some aspects, the vNAR is present at a concentration of at least about 2750 copies per EV. In some aspects, the vNAR is present at a concentration of at least about 3000 copies per EV. In some aspects, the vNAR is present at a concentration of at least about 3250 copies per EV. In some aspects, the vNAR present at a concentration of at least about 3500 copies per EV. In some aspects, the vNAR is present at a concentration of at least about 3750 copies per EV. In some aspects, the vNAR is present at a concentration of at least about 4000 copies per EV. In some aspects, the vNAR is present at a concentration of at least about 4250 copies per EV. In some aspects, the vNAR is present at a concentration of at least about 4500 copies per EV. In some aspects, the vNAR is present at a concentration of at least about 4750 copies per EV. In some aspects, the vNAR is present at a concentration of at least about 5000 copies per EV.

The vNAR can be associated with the external surface of the EV by any means known in the art. In some aspects, the vNAR is fused to scaffold protein, e.g., any scaffold protein disclosed herein. In certain aspects, the vNAR is fused to a Scaffold X protein. In some aspects, each EV comprises at least about 100 vNAR-Scaffold X fusion constructs on the exterior surface of the EV. In some aspects, each EV comprises at least about 250 vNAR-Scaffold X fusion constructs on the exterior surface of the EV. In some aspects, each EV comprises at least about 500 vNAR-Scaffold X fusion constructs on the exterior surface of the EV. In some aspects, each EV comprises at least about 750 vNAR-Scaffold X fusion constructs on the exterior surface of the EV. In some aspects, each EV comprises at least about 1000 vNAR-Scaffold X fusion constructs on the exterior surface of the EV. In some aspects, each EV comprises at least about 1500 vNAR-Scaffold X fusion constructs on the exterior surface of the EV. In some aspects, each EV comprises at least about 2000 vNAR-Scaffold X fusion constructs on the exterior surface of the EV. In some aspects, each EV comprises at least about 2500 vNAR-Scaffold X fusion constructs on the exterior surface of the EV. In some aspects, each EV comprises at least about 3000 vNAR-Scaffold X fusion constructs on the exterior surface of the EV. In some aspects, each EV comprises at least about 3500 vNAR-Scaffold X fusion constructs on the exterior surface of the EV. In some aspects, each EV comprises at least about 4000 vNAR-Scaffold X fusion constructs on the exterior surface of the EV. In some aspects, each EV comprises at least about 4500 vNAR-Scaffold X fusion constructs on the exterior surface of the EV. In some aspects, each EV comprises at least about 5000 vNAR-Scaffold X fusion constructs on the exterior surface of the EV.

II.C. Modified EVs Comprising Tropism Moieties

In some aspects, an EV comprising a vNAR that specifically binds TrF, as disclosed herein, can be further surface engineered to adjust its properties, e.g., biodistribution, e.g., via incorporation of additional immuno-affinity ligands or cognate receptor ligands. For example, EV disclosed herein can be surface engineered to direct them to a specific cellular type, e.g., Schwann cells, sensory neurons, motor neurons, meningeal macrophages, or a tumor cell, or can be surface engineered to enhance their migration to a specific compartment, e.g., to the CNS (in order to improve intrathecal compartment retention) or to a tumor microenvironment.

In some aspects, an EV comprises (i) a vNAR that specifically binds TfR and (ii) a bio-distribution modifying agent or targeting moiety. In some aspects, the bio-distribution modifying agent or targeting moiety comprises a single-domain antigen-biding moiety, e.g., a second vNAR and/or a VHH. As used here, the terms “bio-distribution modifying agent” and “targeting moiety” are used interchangeably and refer to an agent that can modify the distribution of extracellular vesicles (e.g., exosomes, nanovesicles) in vivo or in vitro (e.g., in a mixed culture of cells of different varieties). In some aspects, the targeting moiety alters the tropism of the EV, i.e., the target moiety is a “tropism moiety”. As used herein, the term “tropism moiety” refers to a targeting moiety that when expressed and/or loaded on an EV alters and/or enhances the natural movement of the EV. For example, in some aspects, a tropism moiety can promote the EV to be taken up by a particular cell, tissue, or organ.

EVs exhibit preferential uptake in discrete cell types and tissues, and their tropism can be directed by adding proteins to their surface that interact with receptors on the surface of target cells. The tropism moiety can comprise a biological molecule, such as a protein, a peptide, a lipid, or a carbohydrate, or a synthetic molecule. For example, in some aspects the tropism moiety can comprise an affinity ligand, e.g., an antibody (such as an anti-CD19 nanobody, an anti-CD22 nanobody, an anti-CLEC9A nanobody, or an anti-CD3 nanobody), a VHH domain, a phage display peptide, a fibronectin domain, a camelid nanobody, and/or a vNAR. In some aspects, the tropism moiety can comprise, e.g., a synthetic polymer (e.g., PEG), a natural ligand/molecule (e.g., CD40L, albumin, CD47, CD24, CD55, CD59), and/or a recombinant protein (e.g., XTEN).

In some aspects, a tropism moiety can increase uptake of the EV by a cell. In some aspects, the tropism moiety that can increase uptake of the EV by a cell comprises a lymphocyte antigen 75 (also known as DEC205 or CD205), C-type lectin domain family 9 member A (CLEC9A), C-type lectin domain family 6 (CLEC6), C-type lectin domain family 4 member A (also known as DCIR or CLEC4A), Dendritic Cell-Specific Intercellular adhesion molecule-3-Grabbing Non-integrin (also known as DC-SIGN or CD209), lectin-type oxidized LDL receptor 1(LOX-1), macrophage receptor with collagenous structure (MARCO), C-type lectin domain family 12 member A (CLEC12A), C-type lectin domain family 10 member A (CLEC10A), DC-asialoglycoprotein receptor (DC-ASGPR), DC immunoreceptor 2 (DCIR2), Dectin-1, macrophage mannose receptor (MMR), BDCA-2 (CD303, CLEC4C), Dectin-2, BST-2 (CD317), Langerin, CD206, CD11b, CD11c, CD123, CD304, XCR1, AXL, SIGLEC 6, CD209, SIRPA, CX3CR1, GPR182, CD14, CD16, CD32, CD34, CD38, CD10, anti-CD3 antibody, or any combination thereof.

In some aspects, when tropism to the central nervous system is desired, an EV of the present disclosure can comprise a tissue or cell-specific target ligand, which increases EV tropism to a specific central nervous system tissue or cell. In some aspects, the cell is a glial cell. In some aspects, the glial cell is an oligodendrocyte, an astrocyte, an ependymal cell, a microglia cell, a Schwann cell, a satellite glial cell, an olfactory ensheathing cell, or a combination thereof. In some aspects, the cell is a neural stem cell. In some aspects, the cell-specific target ligand, which increases EV tropism to a Schwann cells binds to a Schwann cell surface marker such as Myelin Basic Protein (MBP), Myelin Protein Zero (P0), P75NTR, NCAM, PMP22, or any combination thereof. In some aspects, the cell-specific tropism moiety comprises an antibody or an antigen-binding portion thereof, an aptamer, or an agonist or antagonist of a receptor expressed on the surface of the Schwann cell.

In some aspects, the bio-distribution modifying agent or targeting moiety comprises an antigen-binding moiety that binds an antigen expressed on a tumor cell. In some aspects, the bio-distribution modifying agent or targeting moiety comprises an antigen-binding moiety that binds an antigen expressed in a tumor microenvironment. In some aspects, the bio-distribution modifying agent or targeting moiety comprises an antigen-binding moiety that binds mesothelin. Any antigen-binding moiety known in the art that is capable of binding mesothelin can be used in the EVs disclosed herein. In some aspects, bio-distribution modifying agent or targeting moiety comprises an antigen-binding moiety that binds CD33. Any antigen-binding moiety known in the art that is capable of binding CD33 can be used in the EVs disclosed herein. In certain aspects, the antigen-binding moiety that binds CD33 is selected from the anti-CD33 binding moieties disclosed in U.S. Pat. No. 5,877,296, which is incorporated by reference herein in its entirety.

In principle, the EVss of the present disclosure comprising a vNAR that specifically binds TfR and at least one additional tropism moiety that can direct the EV to a specific target cell or tissue (e.g., a cell in the CNS or a Schwann cell in peripheral nerves) can be administered using any suitable administration method known in the art (e.g., intravenous injection or infusion) since the presence of the tropism moiety (alone or in combination with the presence of an antiphagocytic signal such as CD47 and the use of a specific administration route) will induce a tropism of the EVs towards the desired target cell or tissue.

In certain aspects, the tropism moiety is linked, e.g., chemically linked via a maleimide moiety, to a scaffold moiety, e.g., a Scaffold X protein or a fragment thereof, on the exterior surface of the EV. Tropism can be further improved by the attachment of an anti-phagocytic signal (e.g., CD47 and/or CD24), a half-life extension moiety (e.g., albumin or PEG), or any combination thereof to the external surface of an EV of the present disclosure. In certain aspects, the anti-phagocytic signal is linked, e.g., chemically linked via a maleimide moiety, to a scaffold moiety, e.g., a Scaffold X protein or a fragment thereof, on the exterior surface of the EV.

Pharmacokinetics, biodistribution, and in particular tropism and retention in the desired tissue or anatomical location can also be accomplished by selecting the appropriate administration route (e.g., intrathecal administration or intraocular administration to improve tropism to the central nervous system), as disclosed herein.

In some aspects, the EV comprises the vNAR that specifically binds TfR and at least two additional different tropism moieties. In some aspects, the EV comprises the vNAR that specifically binds TfR and at least three additional different tropism moieties. In some aspects, the EV comprises the vNAR that specifically binds TfR and at least four additional different tropism moieties. In some aspects, the EV comprises the vNAR that specifically binds TfR and at least five additional or more different tropism moieties. In some aspects, one or more of the tropism moieties increases uptake of the EV by a cell. In some aspects, each tropism moiety is attached to a scaffold moiety, e.g., a Scaffold X protein or a fragment thereof. In some aspects, multiple tropism moieties can be attached to the same scaffold moiety, e.g., a Scaffold X protein or a fragment thereof. In some aspects, several tropism moieties can be attached in tandem to a scaffold moiety, e.g., a Scaffold X protein or a fragment thereof. In some aspects, a tropism moiety disclosed herein or a combination thereof is attached to a scaffold moiety, e.g., a Scaffold X protein or a fragment thereof, via a linker or spacer. In some aspects, a linker or spacer or a combination thereof is interposed between two tropism moieties disclosed herein.

Non-limiting examples of tropism moieties capable of directing EVs of the present disclosure to different nervous system cell types are disclosed below.

II.C.1. Tropism Moieties Targeting Schwann Cells

In some aspects, an additional tropism moiety can target a Schwann cell. In some aspects, the tropism moiety that directs an EV disclosed herein to a Schwann cell targets, e.g., apolipoprotein D (ApoD), Galectin 1 (LGALS1), Myelin proteolipid protein (PLP), Glypican 1, or Syndecan 3. In some aspects, the tropism moiety directing an EV of the present disclosure to a Schwann cell is a transferrin, or a fragment, variant or derivative thereof.

In some aspects, an additional tropism moiety of the present disclosure targets ApoD. Unlike other lipoproteins, which are mainly produced in the liver, apolipoprotein D is mainly produced in the brain, cerebellum, and peripheral nerves. ApoD is 169 amino acids long, including a secretion peptide signal of 20 amino acids. It contains two glycosylation sites (aspargines 45 and 78) and the molecular weight of the mature protein varies from 20 to 32 kDa. ApoD binds steroid hormones such as progesterone and pregnenolone with a relatively strong affinity, and to estrogen with a weaker affinity. Arachidonic acid (AA) is an ApoD ligand with a much better affinity than that of progesterone or pregnenolone. Other ApoD ligands include E-3-methyl-2-hexenoic acid, retinoic acid, sphingomyelin and sphingolipids. Accordingly, in some aspects, a tropism moiety of the present disclosure comprises a ligand that can target ApoD, e.g., an antibody or other binding molecule capable of specifically binding to ApoD.

In some aspects, an additional tropism moiety of the present disclosure targets Galectin 1. The galectin-1 protein is 135 amino acids in length. Accordingly, in some aspects, a tropism moiety of the present disclosure comprises a ligand that can target Galectin 1, e.g., an antibody or other binding molecule capable of specifically binding to Galectin 1.

In some aspects, an additional tropism moiety of the present disclosure targets PLP. PLP is the major myelin protein from the CNS. It plays an important role in the formation or maintenance of the multilamellar structure of myelin. The myelin sheath is a multi-layered membrane, unique to the nervous system that functions as an insulator to greatly increase the efficiency of axonal impulse conduction. PLP is a highly conserved hydrophobic protein of 276 to 280 amino acids which contains four transmembrane segments, two disulfide bonds and which covalently binds lipids (at least six palmitate groups in mammals). Accordingly, in some aspects, a tropism moiety of the present disclosure comprises a ligand that can target PLP, e.g., an antibody or other binding molecule capable of specifically binding to PLP.

In some aspects, an additional tropism moiety of the present disclosure targets Glypican 1. Accordingly, in some aspects, a tropism moiety of the present disclosure comprises a ligand that can target Glypican 1, e.g, an antibody or other binding molecule capable of specifically binding to Glypican 1. In some aspects, a tropism moiety of the present disclosure targets Syndecan 3. Accordingly, in some aspects, a tropism moiety of the present disclosure comprises a ligand that can target Syndecan 3, e.g., an antibody or other binding molecule capable of specifically binding to Syndecan 3.

II.C.2. Tropism Moieties Targeting Sensory Neurons

In some aspects, an additional tropism moiety disclosed herein can direct an EV disclosed herein to a sensory neuron. In some aspects, the tropism moiety that directs an EV disclosed herein to a sensory neuron targets a Trk receptor, e.g., TrkA, TrkB, TrkC, or a combination thereof.

Trk (tropomyosin receptor kinase) receptors are a family of tyrosine kinases that regulates synaptic strength and plasticity in the mammalian nervous system. The common ligands of Trk receptors are neurotrophins, a family of growth factors critical to the functioning of the nervous system. The binding of these molecules is highly specific. Each type of neurotrophin has different binding affinity toward its corresponding Trk receptor. Accordingly, in some aspects, the tropism moiety directing an EV disclosed herein to a sensory neuron, comprises a neurotrophin.

Neurotrophins bind to Trk receptors as homodimers. Accordingly, in some aspects, the tropism moiety comprises at least two neurotrophins disclosed herein, e.g., in tandem. In some aspects, the tropism moiety comprises at least two neurotrophins disclosed herein, e.g., in tandem, that are attached to a scaffold protein, for example, Protein X, via a linker. In some aspects, the linker connecting the scaffold protein, e.g., Protein X, to the neurotrophin (e.g., a neurotrophin homodimer) has a length of at least 10 amino acids. In some aspects, the linker connecting the scaffold protein, e.g., Protein X, to the neurotrophin (e.g., a neurotrophin homodimer) has a length of at least about 25 amino acids, about 30 amino acids, about 35 amino acids, about 40 amino acids, about 45 amino acids, or about 50 amino acids.

In some aspects, the neurotrophin is a neurotrophin precursor, i.e., a proneurotrophin, which is later cleaved to produce a mature protein.

Nerve growth factor (NGF) is the first identified and probably the best characterized member of the neurotrophin family. It has prominent effects on developing sensory and sympathetic neurons of the peripheral nervous system. Brain-derived neurotrophic factor (BDNF) has neurotrophic activities similar to NGF, and is expressed mainly in the CNS and has been detected in the heart, lung, skeletal muscle and sciatic nerve in the periphery (Leibrock, J. et al., Nature, 341:149-152 (1989)). Neurotrophin-3 (NT-3) is the third member of the NGF family and is expressed predominantly in a subset of pyramidal and granular neurons of the hippocampus, and has been detected in the cerebellum, cerebral cortex and peripheral tissues such as liver and skeletal muscles (Ernfors, P. et al., Neuron 1: 983-996 (1990)). Neurotrophin-4 (also called NT-415) is the most variable member of the neurotrophin family. Neurotrophin-6 (NT-5) was found in teleost fish and binds to p75 receptor.

In some aspects, the neurotrophin targeting TrkB comprises, e.g., NT-4 or BDNF, or a fragment, variant, or derivative thereof. In some aspects, the neurotrophin targeting TrkA comprises, e.g., NGF or a fragment, variant, or derivative thereof. In some aspects, the neurotrophin targeting TrkC comprises, e.g., NT-3 or a fragment, variant, or derivative thereof.

In some aspects, the additional tropism moiety comprises brain derived neurotrophic factor (BDNF). In some aspects, the BDNF is a variant of native BDNF, such as a two amino acid carboxyl-truncated variant. In some aspects, the tropism moiety comprises the full-length 119 amino acid sequence of BDNF (HSDPARRGELSVCDSISEWVTAADKKTAVDMSGGTVTVLEKVPVSKGQLKQYFYETK CNPMGYTKEGCRGIDKRHWNSQCRTTQSYVRALTMDSKKRIGWRFIRIDTSCVCTLTIK RGR; SEQ ID NO: 160. In some aspects, a one amino-acid carboxy-truncated variant of BDNF is utilized (amino acids 1-1181 of SEQ ID NO: 161).

In some aspects, the additional tropism moiety comprises a carboxy-truncated variant of the native BDNF, e.g., a variant in which 1, 2, 3, 4, 5, 6, 7, 8, 9, 10, or more than 10 amino acids are absent from the carboxy-terminus of the BDNF. BDNF variants include the complete 119 amino acid BDNF, the 117 or 118 amino acid variant with a truncated carboxyl terminus, variants with a truncated amino terminus, or variants with up to about 20%, about 30, or about 40% change in amino acid composition, as long as the protein variant still binds to the TrkB receptor with high affinity.

In some aspects, the additional tropism moiety comprises a two amino-acid carboxy-truncated variant of BDNF (amino acids 1-117 of SEQ ID NO: 161). In some aspects, the additional tropism moiety comprises a three amino-acid carboxy-truncated variant of BDNF (amino acids 1-116 of SEQ ID NO: 161). In some aspects, the additional tropism moiety comprises a four amino-acid carboxy-truncated variant of BDNF (amino acids 1-115 of SEQ ID NO: 161). In some aspects, the additional tropism moiety comprises a five amino-acid carboxy-truncated variant of BDNF (amino acids 1-114 of SEQ ID NO: 161). In some aspects, the additional tropism moiety comprises a BDNF that is at least about 60%, at least about 65%, at least about 70%, at least about 75%, at least about 80%, at least about 85%, at least about 90%, at least about 95%, at least about 99%, or about 100% identical with the sequence of SEQ ID NO: 161, or a truncated version thereof, e.g., the 117 or 118 amino acid variant with a one- or two-amino acid truncated carboxyl terminus, or variants with a truncated amino terminus. See, e.g., U.S. Pat. No. 8,053,569B2, which is herein incorporated by reference in its entirety.

In some aspects, the additional tropism moiety comprises nerve growth factor (NGF). In some aspects, the NGF is a variant of native NGF, such as a truncated variant. In some aspects, the additional tropism moiety comprises the 26-kDa beta subunit of protein, the only component of the 7S NGF complex that is biologically active. In some aspects, the additional tropism moiety comprises the full-length 120 amino acid sequence of beta NGF (SSSHPIFHRGEFSVCDSVSVWVGDKTTATDIKGKEVMVLGEVNINNSVFKQYFFETKCR DPNPVDSGCRGIDSKHWNSYCTTTHTFVKALTMDGKQAAWRFIRIDTACVCVLSRKAV RRA; SEQ ID NO: 162). In some aspects, the additional tropism moiety comprises a carboxy-truncated variant of the native NGF, e.g., a variant in which 1, 2, 3, 4, 5, 6, 7, 8, 9, 10, or more than 10 amino acids are absent from the carboxy-terminus of NGF. NGF variants include the complete 120 amino acid NGF, the shorter amino acid variants with a truncated carboxyl terminus, variants with a truncated amino terminus, or variants with up to about 20%, about 30%, or about 40% change in amino acid composition, as long as the additional tropism moiety still binds to the TrkB receptor with high affinity. In some aspects, the additional tropism moiety comprises an NGF that is at least about 60%, at least about 65%, at least about 70%, at least about 75%, at least about 80%, at least about 85%, at least about 90%, at least about 95%, at least about 99%, or about 100% identical with the sequence of SEQ ID NO: 162, or a truncated version thereof.

In some aspects, the additional tropism moiety comprises neurotrophin-3 (NT-3). In some aspects, the NT-3 is a variant of native NT-3, such as a truncated variant. In some aspects, the additional tropism moiety comprises the full-length 119 amino acid sequence of NT-3 (YAEHKSHRGEYSVCDSESLWVTDKSSAIDIRGHQVTVLGEIKTGNSPVKQYFYETRCKE ARPVKNGCRGIDDKHWNSQCKTSQTYVRALTSENNKLVGWRWIRIDTSCVCALSRKIG RT; SEQ ID NO: 163). In some aspects, the additional tropism moiety comprises a carboxy-truncated variant of the native NT-3, e.g., a variant in which 1, 2, 3, 4, 5, 6, 7, 8, 9, 10, or more than 10 amino acids are absent from the carboxy-terminus of NT-3. NT-3 variants include the complete 119 amino acid NT-3, the shorter amino acid variants with a truncated carboxyl terminus, variants with a truncated amino terminus, or variants with up to about 20%, about 30%, or about 40% change in amino acid composition, as long as the tropism moiety still binds to the TrkC receptor with high affinity. In some aspects, the additional tropism moiety comprises an NT-3 that is at least about 60%, at least about 65%, at least about 70%, at least about 75%, at least about 80%, at least about 85%, at least about 90%, at least about 95%, at least about 99%, or about 100% identical with the sequence of SEQ ID NO: 163, or a truncated version thereof.

In some aspects, the additional tropism moiety comprises neurotrophin-4 (NT-4). In some aspects, the NT-4 is a variant of native NT-4, such as a truncated variant. In some aspects, the tropism moiety comprises the full-length 130 amino acid sequence of NT-4 (GVSETAPASRRGELAVCDAVSGWVTDRRTAVDLRGREVEVLGEVPAAGGSPLRQYFFE TRCKADNAEEGGPGAGGGGCRGVDRRHWVSECKAKQSYVRALTADAQGRVGWRWIR IDTACVCTLLSRTGRA; SEQ ID NO: 164). In some aspects, the additional tropism moiety comprises a carboxy-truncated variant of the native NT-4, e.g., a variant in which 1, 2, 3, 4, 5, 6, 7, 8, 9, 10, or more than 10 amino acids are absent from the carboxy-terminus of NT-4. NT-4 variants include the complete 130 amino acid NT-4, the shorter amino acid variants with a truncated carboxyl terminus, variants with a truncated amino terminus, or variants with up to about 20%, about 30%, or about 40% change in amino acid composition, as long as the tropism moiety still binds to the TrkB receptor with high affinity. In some aspects, the additional tropism moiety comprises an NT-4 that is at least about 60%, at least about 65%, at least about 70%, at least about 75%, at least about 80%, at least about 85%, at least about 90%, at least about 95%, at least about 99%, or about 100% identical with the sequence of SEQ ID NO: 164, or a truncated version thereof.

Structure/function relationship studies of NGF and NGF-related recombinant molecules demonstrated that mutations in NGF region 25-36, along with other β-hairpin loop and non-loop regions, significantly influenced NGF/NGF-receptor interactions (Ibanez et al., EMBO J., 10, 2105-2110, (1991)). Small peptides derived from this region have been demonstrated to mimic NGF in binding to Mock receptor and affecting biological responses (LeSauteur et al. J. Biol. Chem. 270, 6564-6569, 1995). Dimers of cyclized peptides corresponding to β-loop regions of NGF were found to act as partial NGF agonists in that they had both survival-promoting and NGF-inhibiting activity while monomer and linear peptides were inactive (Longo et al., J. Neurosci. Res., 48, 1-17, 1997). Accordingly, in some aspects, a tropism moiety of the present disclosure comprises such peptides.

Cyclic peptides have also been designed and synthesized to mimic the β-loop regions of NGF, BDNF, NT3 and NT-4/5. Certain monomers, dimers or polymers of these cyclic peptides can have a three-dimensional structure, which binds to neurotrophin receptors under physiological conditions. All of these structural analogs of neurotrophins that bind to nerve cell surface receptors and are internalized can serve as the binding agent B of the compound according to the present disclosure to deliver the conjugated therapeutic moiety TM to the nervous system. Accordingly, in some aspects, an additional tropism moiety of the present disclosure comprises such cyclic peptides or combinations thereof.

In some aspects, antibodies against nerve cell surface receptors that are capable of binding to the receptors and being internalized can also serve as tropism moieties binding to a Trk receptor. For example, monoclonal antibody (MAb) 5C3 is specific for the NGF docking site of the human p140 TrkA receptor, with no cross-reactivity with human TrkB receptor. MAb 5C3 and its Fab mimic the effects of NGF in vitro, and image human Trk-A positive tumors in vivo (Kramer et al., Eur. J. Cancer, 33, 2090-2091, (1997)). Molecular cloning, recombination, mutagenesis and modeling studies of Mab 5C3 variable region indicated that three or less of its complementarity determining regions (CDRs) are relevant for binding to TrkA. Assays with recombinant CDRs and CDR-like synthetic polypeptides demonstrated that they had agonistic bioactivities similar to intact Mab 5C3. Monoclonal antibody MC192 against p75 receptor has also been demonstrated to have neurotrophic effects. Therefore, these antibodies and their functionally equivalent fragments can also serve as tropism moieties of the present disclosure.

In some aspects, peptidomimetics that are synthesized by incorporating unnatural amino acids or other organic molecules can also serve tropism moieties of the present disclosure.

Other neurotrophins are known in the art. Accordingly, in some aspects, the target moiety comprises a neurotrophin selected from the group consisting of fibroblast growth factor (FGF)-2 and other FGFs, erythropoietin (EPO), hepatocyte growth factor (HGF), epidermal growth factor (EGF), transforming growth factor (TGF)-a, TGF-(3, vascular endothelial growth factor (VEGF), interleukin-1 receptor antagonist (IL-lra), ciliary neurotrophic factor (CNTF), glial-derived neurotrophic factor (GDNF), neurturin, platelet-derived growth factor (PDGF), heregulin, neuregulin, artemin, persephin, interleukins, granulocyte-colony stimulating factor (CSF), granulocyte-macrophage-CSF, netrins, cardiotrophin-1, hedgehogs, leukemia inhibitory factor (LIF), midlcine, pleiotrophin, bone morphogenetic proteins (BMPs), netrins, saposins, semaphorins, and stem cell factor (SCF).

In some aspects, the additional tropism moiety directing an EV disclosed herein to a sensory neuron, comprises a varicella zoster virus (VZV) peptide.

II.C.3. Tropism Moieties Targeting Motor Neurons

In some aspects, an additional tropism moiety disclosed herein can direct an EV disclosed herein to a motor neuron. In some aspects, the tropism moiety that directs an EV disclosed herein to a motor comprises a Rabies Virus Glycoprotein (RVG) peptide, a Targeted Axonal Import (TAxI) peptide, a P75R peptide, or a Tet-C peptide.

In some aspects, the additional tropism moiety comprises a Rabies Virus Glycoprotein (RVG) peptide. See, e.g., U.S. Pat. App. Publ. 2014-00294727, which is herein incorporated by reference in its entirety. In some aspects, the RVG peptide comprises amino acid residues 173-202 of the RVG (YTIWMPENPRPGTPCDIFTNSRGKRASNG; SEQ ID NO: 165) or a variant, fragment, or derivative thereof. In some aspects, the tropism moiety is a fragment of SEQ ID NO: 165. Such a fragment of SEQ ID NO: 165 can have, for example, 1, 2, 3, 4, 5, 6, 7, 8, 9, or 10 amino acids deleted from the N-terminal and/or the C-terminal of SEQ ID NO: 165. A functional fragment derived from SEQ ID NO: 165 can be identified by sequentially deleting N- and/or C-terminal amino acids from SEQ ID NO: 165 and assessing the function of the resulting peptide fragment, such as function of the peptide fragment to bind acetylcholine receptor and/or ability to transmit through the blood brain barrier. In some aspects, the tropism moiety comprises a fragment of SEQ ID NO: 165 28, 27, 26, 25, 24, 23, 22, 21, 20, 19, 18, 17, 16 or 15 amino acids in length. In some aspects, the tropism moiety comprises a fragment of SEQ ID NO: 165 less than 15 peptides in length.

A “variant” of a RGV peptide, for example SEQ ID NO: 165, is meant to refer to a molecule substantially similar in structure and function, i.e., where the function is the ability to pass or transit through the BBB, to either the entire molecule, or to a fragment thereof. A variant of an RVG peptide can contain a mutation or modification that differs from a reference amino acid in SEQ ID NO: 165. In some aspects, a variant of SEQ ID NO: 165 is a fragment of SEQ ID NO: 165 as disclosed herein. In some aspects, an RVG variant can be a different isoform of SEQ ID NO: 165 or can comprise different isomer amino acids. Variants can be naturally-occurring, synthetic, recombinant, or chemically modified polynucleotides or polypeptides isolated or generated using methods well known in the art. RVG variants can include conservative or non-conservative amino acid changes. See, e.g., U.S. Pat. No. 9,757,470, which is herein incorporated by reference in its entirety.

In some aspects, the additional tropism moiety comprises a Targeted Axonal Import (TAxI) peptide. In some aspects, the TAxI peptide is cyclized TAxI peptide of sequence SACQSQSQMRCGGG (SEQ ID NO: 166). See, e.g., Sellers et al. (2016) Proc. Natl. Acad. Sci. USA 113:2514-2519, and U.S. Pat. No. 9,056,892, which are herein incorporated by reference in their entireties. TAxI transport peptides as described herein may be of any length. Typically, the transport peptide will be between 6 and 50 amino acids in length, more typically between 10 and 20 amino acids in length. In some aspects, the TAxI transport peptide comprises the amino acid sequence QSQSQMR (SEQ ID NO: 167), ASGAQAR (SEQ ID NO: 168). PE, or TSTAPHLALRTSR (SEQ 11) NO: 169). Optionally, the TAxI transport peptide further includes a flanking sequence to facilitate incorporation into a delivery construct or carrier, e.g., a linker. In one aspect, the peptide is flanked with cysteines. In some aspects, the TAxI transport peptide further comprises additional sequence selected to facilitate delivery into nuclei. For example, a peptide that facilitates nuclear delivery is a nuclear localizing signal (NLS). Typically, this signal consists of a few short sequences of positively charged lysines or arginines, such as PPKKRKV (SEQ ID NO: 170). In one aspect, the NLS has the amino acid sequence PKKRKV (SEQ ID NO: 171).

In some aspects, an additional tropism moiety of the present disclosure comprises a peptide BBB shuttle disclosed in the table below. See, e.g., Oller-Salvia et al. (2016) Chem. Soc. Rev. 45, 4690-4707, and Jafari et al. (2019) Expert Opinion on Drug Delivery 16:583-605 which are herein incorporated by reference in their entireties.

| SEQ ID | | |
|---|---|---|
| NO | Peptide | Sequence |
| 172 | Angiopep-2 | TFFYGGSRGKRNNFKTEEY-OH |
| 173 | ApoB (3371-3409) | SSVIDALQYKLEGTTRLTRK- |
| | | RGLKLATALSLSNKFVEGS |
| 174 | ApoE (159-167)₂ | (LRKLRKRLL)₂ |
| 175 | Peptide-22 | Ac-C(&)MPRLRGC(&)-NH₂ |
| 176 | THR | THRPPMWSPVWP-NH₂ |
| 177 | THR retro- | pwvpswmpprht-NH₂ |
| | enantio | |
| 178 | CRT | C(&)RTIGPSVC(&) |
| 179 | Leptin30 | YQQILTSMPSRNVIQISND- |
| | | LENLRDLLHVL |
| 180 | RVG29 | YTIWMPENPRPGTPCDIFT- |
| | | NSRGKRASNG-OH |
| 181 | ^{D}CDX | GreirtGraerwsekf-OH |
| 182 | Apamin | C(&₁)NC(&₂)KAPETALC(&₁)- |
| | | AR-RC(&₂)QQH-NH₂ |
| 183 | MiniAp-4 | [Dap](&)KAPETALD(&) |
| 184 | GSH | γ-L-glutamyl-CG-OH |
| 185 | G23 | HLNILSTLWKYRC |
| 186 | g7 | GFtGFLS(O-β-Glc)-NH₂ |
| 187 | TGN | TGNYKALHPHNG |
| 188 | TAT(47-57) | YGRKKRRQRRR-NH₂ |
| 189 | SynB1 | RGGRLSYSRRRFSTSTGR |
| 190 | Diketopiperazines | &(N-MePhe)-(N-MePhe) |
| | | Diketo-piperazines |
| 191 | PhPro | (Phenylproline)₄-NH₂ |

Nomenclature for cyclic peptides (&) is adapted to the 3-letter amino acid code from the one described by Spengler et al. Pept. Res., 2005, 65, 550-555
[Dap] stands for diaminopropionic acid.

II.D. Biologically Active Moiety

In some aspects, the EVs disclosed herein comprise (i) a vNAR that specifically binds TfR and (ii) one or more payloads, e.g., one or more biologically active moieties. Payloads (e.g., biologically active moieties) comprise a small molecule, a therapeutic protein, an antigen, an adjuvant, an immune modulator, or any commination thereof.

In some aspects, an EV disclosed herein is capable of delivering a payload (a biologically active molecule attached to the EV) to a target. The payload is an agent that acts on a target (e.g., a target cell) that is contacted with the EV. Contacting can occur in vitro or in a subject. Non-limiting examples of payloads that can attached to an EV via a maleimide moiety include agents such as, nucleotides (e.g., nucleotides comprising a detectable moiety or a toxin or that disrupt transcription), nucleic acids (e.g., DNA or mRNA molecules that encode a polypeptide such as an enzyme, or RNA molecules that have regulatory function such as miRNA, dsDNA, lncRNA, or siRNA), amino acids (e.g., amino acids comprising a detectable moiety or a toxin that disrupt translation), polypeptides (e.g., enzymes), lipids, carbohydrates, and small molecules (e.g., small molecule drugs and toxins). In some aspects, a payload is in the lumen of the EV.

In some aspects, the vNAR is fused to the external surface of the EV as disclosed herein, and the payload is fused to the external surface of the EV. In some aspects, the vNAR is fused to the external surface of the EV as disclosed herein, and the payload is fused to the luminal surface of the EV. In some aspects, the vNAR is fused to the external surface of the EV as disclosed herein, and the payload is present in solution in the lumen of the EV. In some aspects, an EV can comprise more than one payload, e.g., a first payload in solution in the lumen of EV, and a second payload attached, e.g., to the external surface of the EV via a maleimide moiety.

In some aspects, the payload is a small molecule. In some aspects, the small molecule is a proteolysis-targeting chimera (PROTAC).

In some aspects, the payload comprises a nucleotide, wherein the nucleotide is a stimulator of interferon genes protein (STING) agonist. STING is a cytosolic sensor of cyclic dinucleotides that is typically produced by bacteria. Upon activation, it leads to the production of type I interferons and initiates an immune response

In some aspects, the EV of the present disclosure further comprises one or more STING agonists covalently linked to the EV via a maleimide moiety. In some aspects, the STING agonist comprises a cyclic nucleotide STING agonist or a non-cyclic dinucleotide STING agonist.

In some aspects, the payload (e.g., a biologically active moiety) is a TLR agonist. Non-limiting examples of TLR agonists include: TLR2 agonist (e.g., lipoteichoic acid, atypical LPS, MALP-2 and MALP-404, OspA, porin, LcrV, lipomannan, GPI anchor, lysophosphatidylserine, lipophosphoglycan (LPG), glycophosphatidylinositol (GPI), zymosan, hsp60, gH/gL glycoprotein, hemagglutinin), a TLR3 agonist (e.g., double-stranded RNA, e.g., poly(I:C)), a TLR4 agonist (e.g., lipopolysaccharides (LPS), lipoteichoic acid, β-defensin 2, fibronectin EDA, HMGB1, snapin, tenascin C), a TLR5 agonist (e.g., flagellin), a TLR6 agonist, a TLR7/8 agonist (e.g., single-stranded RNA, CpG-A, Poly G10, Poly G3, Resiquimod), a TLR9 agonist (e.g., unmethylated CpG DNA), and combinations thereof. Non-limiting examples of TLR agonists can be found at WO2008115319A2, US20130202707A1, US20120219615A1, US20100029585A1, WO2009030996A1, WO2009088401A2, and WO2011044246A1, each of which are incorporated by reference in its entirety.

In some aspects, the payload is an antibody or antigen binding fragment thereof. In some aspects, the payload is an ADC. In some aspects, the payload is a small molecule comprising a synthetic antineoplastic agent (e.g., monomethyl auristatin E (MMAE) (vedotin)), a cytokine release inhibitor (e.g., MCC950), an mTOR inhibitor (e.g. Rapamycin and its analogs (Rapalogs)), an autotaxin inhibitor (e.g., PAT409), a lysophosphatidic acid receptor antagonist (e.g., BMS-986020), or any combination thereof.

In some aspects, the therapeutic protein comprises a clotting factor. In some aspects, the clotting factor is selected from the group consisting of FI, FII, FIII, FIV, FV, FVI, FVII, FVIII, FIX, FX, FXI, FXII, FXIII), VWF, prekallikrein, high-molecular weight kininogen, fibronectin, antithrombin III, heparin cofactor II, protein C, protein S, protein Z, Protein Z-related protease inhibitor (ZPI), plasminogen, alpha 2-antiplasmin, tissue plasminogen activator (tPA), urokinase, plasminogen activator inhibitor-1 (PAI-1), plasminogen activator inhibitor-2 (PAI2), any zymogen thereof, any active form thereof, and any combination thereof. In some aspects, the clotting factor comprises FVIII or a variant or fragment thereof. In another embodiment, the clotting factor comprises FIX or a variant or fragment thereof. In another embodiment, the clotting factor comprises FVII or a variant or fragment thereof. In another embodiment, the clotting factor comprises VWF or a variant or fragment thereof.

In some aspects, the therapeutic protein comprises a growth factor. The growth factor can be selected from any growth factor known in the art. In some aspects, the growth factor is a hormone. In some aspects, the growth factor is a cytokine. In some aspects, the growth factor is a chemokine.

II.D.1. Oligonucleotides

In some aspects, the payload is an antisense oligonucleotide, a phosphorodiamidate Morpholino oligomer (PMO), or a peptide-conjugated phosphorodiamidate morpholino oligomer (PPMO), an antisense oligonucleotide (ASO), a siRNA, a miRNA, a shRNA, a nucleic acid, or any combination thereof. In some aspects, the payload is a fusogenic peptide.

In some aspects, the ASO is targets PMP22. In humans, the PMP22 gene is located on chromosome 17p11.2 and spans approximately 40 kb. The gene contains six exons conserved in both humans and rodents, two of which are 5′ untranslated exons (1a and 1b) and result in two different RNA transcripts with identical coding sequences. The two transcripts differ in their 5′ untranslated regions and have their own promoter regulating expression. The remaining exons (2 to 5) include the coding region of the PMP22 gene, and are joined together after post-transcriptional modification (i.e. alternative splicing). The PMP22 protein is characterized by four transmembrane domains, two extracellular loops (ECL1 and ECL2), and one intracellular loop. ECL1 has been suggested to mediate a homophilic interaction between two PMP22 proteins, whereas ECL2 has been shown to mediate a heterophilic interaction between PMP22 protein and Myelin protein zero (MPZ or MPO).

Improper gene dosage of the PMP22 gene can cause aberrant protein synthesis and function of myelin sheath. Since the components of myelin are stoichiometrically set, any irregular expression of a component can cause destabilization of myelin and neuropathic disorders. Alterations of PMP22 gene expression are associated with a variety of neuropathies, such as Charcot-Marie-Tooth type 1A (CMT1A), Dejerine-Sottas disease, and Hereditary Neuropathy with Liability to Pressure Palsy (HNPP). Too much PMP22 (e.g. caused by gene duplication) results in CMT1A. Gene duplication of PMP22 is the most common genetic cause of CMT where the overproduction of PMP22 results in defects in multiple signaling pathways and dysfunction of transcriptional factors like KNOX20, SOX10 and EGR2.

The sequence for the human PMP22 gene can be found under publicly available as NCBI RefSeq Acc. No. NM_000304. Alternative RefSeq mRNA transcripts have accession numbers NM_001281455, NM-001281456, NM-153321, and NM_153322, respectively. The human PMP22 gene is found at chromosome location 17p12 at 15,229,777-15,265,326.

The sequence for the human PMP22 pre-mRNA transcript corresponds to the reverse complement of residues 15,229,777-15,265,326, of chromosome location 17p12. The sequence for human PMP22 protein can be found under publicly available Uniprot Accession Number Q01453. Potential PMP22 isoforms have Uniprot Accession Numbers A8MU75, J3KQW0, A0A2R8Y5L5, J3KT36, and J3Q508, respectively. The publicly available contents of the database entries corresponding to accession numbers disclosed herein are incorporated by reference in their entireties.

In some aspects, the ASO targets a transcript, which is a STAT6 transcript, a CEBP/β transcript, a STAT3 transcript, a KRAS transcript, a NRAS transcript, an NLPR3 transcript, or any combination thereof.

STAT6 (STAT6) is also known as signal transducer and activator of transcription 6. Synonyms of STATE/STAT6 are known and include IL-4 STAT; STAT, Interleukin4-Induced; Transcription Factor IL-4 STAT; STAT6B; STAT6C; and D1251644. The sequence for the human STAT6 gene can be found under publicly available GenBank Accession Number NC_000012.12:c57111413-57095404. The human STAT6 gene is found at chromosome location 12q13.3 at 57111413-57095404, complement.

CEBP/β (CEBP/β) is also known as CCAAT/enhancer-binding protein beta. Synonyms of CEBP/β/CEBP/β are known and include C/EBP beta; Liver activator protein; LAP; Liver-enriched inhibitory protein; LIP; Nuclear factor NF-IL6; transcription factor 5; TCF-5; CEBPB; CEBPb; CEBP/3; CEBP/B; and TCF5. The sequence for the human CEBP/β gene can be found under publicly available GenBank Accession Number NC_000020.11 (50190583 . . . 50192690). The human CEBP/β gene is found at chromosome location 20q13.13 at 50190583-50192690.

NRas is an oncogene encoding a membrane protein that shuttles between the Golgi apparatus and the plasma membrane. NRas-encoding genomic DNA can be found at Chromosomal position 1p13.2 (i.e., nucleotides 5001 to 17438 of GenBank Accession No. NG 007572). N-ras mutations have been described in melanoma, thyroid carcinoma, teratocarcinoma, fibrosarcoma, neuroblastoma, rhabdomyosarcoma, Burkitt lymphoma, acute promyelocytic leukemia, T cell leukemia, and chronic myelogenous leukemia. Oncogenic N-Ras can induce acute myeloid leukemia (AML)—or chronic myelomonocytic leukemia (CMML)—like disease in mice. Neuroblastoma RAS viral oncogene (NRas) is known in the art by various names. Such names include: GTPase NRas, N-ras protein part 4, neuroblastoma RAS viral (v-ras) oncogene homolog neuroblastoma RAS viral oncogene homolog, transforming protein N-Ras, and v-ras neuroblastoma RAS viral oncogene homolog.

Signal Transducer and Activator of Transcription 3 (STAT3) is a signal transducer and activator of transcription that transmits signals from cell surface receptors to the nucleus. STAT3 is frequently hyperactivated in many human cancers. STAT3-encoding genomic DNA can be found at Chromosomal position 17q21.2 (i.e., nucleotides 5,001 to 80,171 of GenBank Accession No. NG 007370.1) NLRP3 (NLRP3) is also known as NLR family pyrin domain containing 3. Synonyms of NLRP3/NLRP3 are known and include NLRP3; Clorf7; CIAS1; NALP3; PYPAFJ; nucleotide-binding oligomerization domain, leucine rich repeat and pyrin domain containing 3; cold-induced autoinflammatory syndrome 1 protein; cryopyin; NACHT, LRR and PYD domains-containing protein 3; angiotensin/vasopressin receptor AII/AVP-like; caterpillar protein 1.1; CLR1.1; cold-induced autoinflammatory syndrome 1 protein; and PYRIN-containing APAF1-like protein 1. The sequence for the human NLRP3 gene can be found under publicly available GenBank Accession Number NC_000001.11:247416156-247449108. The human NLRP3 gene is found at chromosome location 1q44 at 247,416,156-247,449,108.

KRAS is known in the art by various names. Such names include: KRAS Proto-Oncogene, GTPase; V-Ki-Ras2 Kirsten Rat Sarcoma 2 Viral Oncogene Homolog; GTPase KRas; C-Ki-Ras; K-Ras 2; KRAS2; RASK2; V-Ki-Ras2 Kirsten Rat Sarcoma Viral Oncogene Homolog; Kirsten Rat Sarcoma Viral Proto-Oncogene; Cellular Transforming Proto-Oncogene; Cellular C-Ki-Ras2 Proto-Oncogene; Transforming Protein P21; PR310 C-K-Ras Oncogene; C-Kirsten-Ras Protein; K-Ras P21 Protein; and Oncogene KRAS2. The sequence for the human KRAS gene can be found at chromosomal location 12p12.1 and under publicly available GenBank Accession Number NC_000012 (25,204,789-25,250,936). The genomic sequence for human wild-type KRAS transcript corresponds to the reverse complement of residues 25,204,789-25,250,936 of NC_000012

II.D.2. Macrophage-Targeting Biologically Active Molecules

In some aspects, the EV comprises (i) a vNAR that specifically binds TfR and (ii) a biologically active moiety that targets macrophages. In other aspects, the biologically active molecule induces macrophage polarization. Macrophage polarization is a process by which macrophages adopt different functional programs in response to the signals from their microenvironment. This ability is connected to their multiple roles in the organism: they are powerful effector cells of innate immune system, but also important in removal of cellular debris, embryonic development and tissue repair.

By simplified classification, macrophage phenotype has been divided into 2 groups: M1 (classically activated macrophages) and M2 (alternatively activated macrophages). This broad classification was based on in vitro studies, in which cultured macrophages were treated with molecules that stimulated their phenotype switching to particular state. In addition to chemical stimulation, it has been shown that the stiffness of the underlying substrate a macrophage is grown on can direct polarization state, functional roles and migration mode. M1 macrophages were described as the pro-inflammatory type, important in direct host-defense against pathogens, such as phagocytosis and secretion of pro-inflammatory cytokines and microbicidal molecules. M2 macrophages were described to have quite the opposite function: regulation of the resolution phase of inflammation and the repair of damaged tissues. Later, more extensive in vitro and ex vivo studies have shown that macrophage phenotypes are much more diverse, overlapping with each other in terms of gene expression and function, revealing that these many hybrid states form a continuum of activation states which depend on the microenvironment. Moreover, in vivo, there is a high diversity in gene expression profile between different populations of tissue macrophages. Macrophage activation spectrum is thus considered to be wider, involving complex regulatory pathway to response to plethora of different signals from the environment. The diversity of macrophage phenotypes still remain to be fully characterized in vivo.

The imbalance of the macrophage types is related to a number of immunity-related diseases. For example, increased M1/M2 ratio can correlate with development of inflammatory bowel disease, as well as obesity in mice. On the other side, in vitro experiments implicated M2 macrophages as the primary mediators of tissue fibrosis. Several studies have associated the fibrotic profile of M2 macrophages with the pathogenesis of systemic sclerosis. Non-limiting examples of the macrophage targeting biologically active molecules are: PI3Ky (phosphatidylinositol-4,5-bisphosphate 3-kinase catalytic subunit gamma), RIP1 (Receptor Interacting Protein (RIP) kinase 1, RIPK1), HIF-1α (Hypoxia-inducible factor 1-alpha), AHR1 (Adhesion and hyphal regulator 1), miR146a, miR155, IRF4 (Interferon regulatory factor 4), PPARγ (Peroxisome proliferator-activated receptor gamma), IL-4RA (Interleukin-4 receptor subunit alpha), TLR8 (Toll-like receptor 8), and TGF-β1 (Transforming growth factor beta-1 proprotein).

II.D.3. Immunomodulators

In some aspects, a biologically active moiety comprises an immune modulator. In some aspects, an immune modulator comprises an inhibitor for a negative checkpoint regulator or an inhibitor for a binding partner of a negative checkpoint regulator. In certain aspects, the negative checkpoint regulator comprises cytotoxic T-lymphocyte-associated protein 4 (CTLA-4), programmed cell death protein 1 (PD-1), lymphocyte-activated gene 3 (LAG-3), T-cell immunoglobulin mucin-containing protein 3 (TIM-3), B and T lymphocyte attenuator (BTLA), T cell immunoreceptor with Ig and ITIM domains (TIGIT), V-domain Ig suppressor of T cell activation (VISTA), adenosine A2a receptor (A2aR), killer cell immunoglobulin like receptor (KIR), indoleamine 2,3-dioxygenase (IDO), CD20, CD39, CD73, or any combination thereof.

In some aspects, the immune modulator is an inhibitor of cytotoxic T-lymphocyte-associate protein 4 (CTLA-4). In certain aspects, the CTLA-4 inhibitor is a monoclonal antibody of CTLA-4 (“anti-CTLA-4 antibody”). In certain aspects, the inhibitor is a fragment of a monoclonal antibody of CTLA-4. In certain aspects, the antibody fragment is a scFv, (scFv)₂, Fab, Fab′, and F(ab′)₂, F(ab1)₂, Fv, dAb, or Fd of a monoclonal antibody of CTLA-4. In certain aspects, the inhibitor is a nanobody, a bispecific antibody, or a multispecific antibody against CTLA-4. In some aspects, the anti-CTLA-4 antibody is ipilimumab. In other aspects, the anti-CTLA-4 antibody is tremelimumab.

In some aspects, the immune modulator is an inhibitor of programmed cell death protein 1 (PD-1). In some aspects, the immune modulator is an inhibitor of programmed death-ligand 1 (PD-L1). In some aspects, the immune modulator is an inhibitor of programmed death-ligand 2 (PD-L2). In certain aspects, the inhibitor of PD-1, PD-L1, or PD-L2 is a monoclonal antibody of PD-1 (“anti-PD-1 antibody”), PD-L1 (“anti-PD-L1 antibody”), or PD-L2 (“anti-PD-L2 antibody”). In some aspects, the inhibitor is a fragment of an anti-PD-1 antibody, anti-PD-L1 antibody, or anti-PD-L2 antibody. In certain aspects, the antibody fragment is a scFv, (scFv)₂, Fab, Fab′, and F(ab′)₂, F(ab1)₂, Fv, dAb, or Fd of a monoclonal antibody of PD-1, PD-L1, or PD-L2. In certain aspects, the inhibitor is a nanobody, a bispecific antibody, or a multispecific antibody against PD-1, PD-L1, or PD-L2. In some aspects, the anti-PD-1 antibody is nivolumab. In some aspects, the anti-PD-1 antibody is pembrolizumab. In some aspects, the anti-PD-1 antibody is pidilizumab. In some aspects, the anti-PD-L1 antibody is atezolizumab. In other aspects, the anti-PD-L1 antibody is avelumab.

In some aspects, the immune modulator is an inhibitor of lymphocyte-activated gene 3 (LAG3). In certain aspects, the inhibitor of LAG3 is a monoclonal antibody of LAG3 (“anti-LAG3 antibody”). In some aspects, the inhibitor is a fragment of an anti-LAG3 antibody, e.g., scFv, (scFv)₂, Fab, Fab′, and F(ab′)₂, F(ab1)₂, Fv, dAb, or Fd. In certain aspects, the inhibitor is a nanobody, a bispecific antibody, or a multispecific antibody against LAG3.

In some aspects, the immune modulator is an inhibitor of T-cell immunoglobulin mucin-containing protein 3 (TIM-3). In some aspects, the immune modulator is an inhibitor of B and T lymphocyte attenuator (BTLA). In some aspects, the immune modulator is an inhibitor of T cell immunoreceptor with Ig and ITIM domains (TIGIT). In some aspects, the immune modulator is an inhibitor of V-domain Ig suppressor of T cell activation (VISTA). In some aspects, the immune modulator is an inhibitor of adenosine A2a receptor (A2aR). In some aspects, the immune modulator is an inhibitor of killer cell immunoglobulin like receptor (KIR). In some aspects, the immune modulator is an inhibitor of indoleamine 2,3-dioxygenase (IDO). In some aspects, the immune modulator is an inhibitor of CD20, CD39, or CD73.

In some aspects, the immune modulator comprises an activator for a positive co-stimulatory molecule or an activator for a binding partner of a positive co-stimulatory molecule. In certain aspects, the positive co-stimulatory molecule comprises a TNF receptor superfamily member (e.g., CD120a, CD120b, CD18, OX40, CD40, Fas receptor, M68, CD27, CD30, 4-1BB, TRAILR1, TRAILR2, TRAILR3, TRAILR4, RANK, OCIF, TWEAK receptor, TACI, BAFF receptor, ATAR, CD271, CD269, AITR, TROY, CD358, TRAMP, and XEDAR). In some aspects, the activator for a positive co-stimulatory molecule is a TNF superfamily member (e.g., TNFα, TNF-C, OX40L, CD40L, FasL, LIGHT, TL1A, CD27L, Siva, CD153, 4-1BB ligand, TRAIL, RANKL, TWEAK, APRIL, BAFF, CAMLG, NGF, BDNF, NT-3, NT-4, GITR ligand, and EDA-2).

In some aspects, the immune modulator is an activator of TNF Receptor Superfamily Member 4 (OX40). In certain aspects, the activator of OX40 is an agonistic anti-OX40 antibody. In further aspects, the activator of OX40 is a OX40 ligand (OX40L).

In some aspects, the immune modulator is an activator of CD27. In certain aspects, the activator of CD27 is an agonistic anti-CD27 antibody. In other aspects, the activator of CD27 is a CD27 ligand (CD27L).

In some aspects, the immune modulator is an activator of CD40. In certain aspects, the activator of CD40 is an agonistic anti-CD40 antibody. In some aspects, the activator of CD40 is a CD40 ligand (CD40L). In certain aspects, the CD40L is a monomeric CD40L. In other aspects, the CD40L is a trimeric CD40L.

In some aspects, the immune modulator is an activator of glucocorticoid-induced TNFR-related protein (GITR). In certain aspects, the activator of GITR is an agonistic anti-GITR antibody. In other aspects, the activator of GITR is a natural ligand of GITR.

In some aspects, the immune modulator is an activator of 4-1BB. In specific aspects, the activator of 4-1BB is an agonistic anti-4-1BB antibody. In certain aspects, the activator of 4-1BB is a natural ligand of 4-1BB.

In some aspects, the immune modulator is a Fas receptor (Fas). In such aspects, the Fas receptor is displayed on the surface of the EV. In some aspects, the immune modulator is Fas ligand (FasL). In certain aspects, the Fas ligand is displayed on the surface of the EV. In some aspects, the immune modulator is an anti-Fas antibody or an anti-FasL antibody.

In some aspects, the immune modulator is an activator of a CD28-superfamily co-stimulatory molecule. In certain aspects, the CD28-superfamily co-stimulatory molecule is ICOS or CD28. In certain aspects, the immune modulator is ICOSL, CD80, or CD86.

In some aspects, the immune modulator is an activator of inducible T cell co-stimulator (ICOS). In certain aspects, the activator of ICOS is an agonistic anti-ICOS antibody. In other aspects, the activator of ICOS is a ICOS ligand (ICOSL).

In some aspects, the immune modulator is an activator of CD28. In some aspects, the activator of CD28 is an agonistic anti-CD28 antibody. In other aspects, the activator of CD28 is a natural ligand of CD28. In certain aspects, the ligand of CD28 is CD80.

In some aspects, the immune modulator comprises a cytokine or a binding partner of a cytokine. In certain aspects, the cytokine comprises IL-2, IL-4, IL-7, IL-10, IL-12, IL-15, IL-21, or IFN-γ. In some aspects, the immune modulator comprises FLT-3 (CD135).

II.D.4. Tumor Antigens

In some aspects, the payload targets a tumor antigen. Non-limiting examples of tumor antigens include: alpha-fetoprotein (AFP), carcinoembryonic antigen (CEA), epithelial tumor antigen (ETA), mucin 1 (MUC1), Tn-MUC1, mucin 16 (MUC16), tyrosinase, melanoma-associated antigen (MAGE), tumor protein p53 (p53), CD4, CD8, CD45, CD80, CD86, programmed death ligand 1 (PD-L1), programmed death ligand 2 (PD-L2), NY-ESO-1, PSMA, TAG-72, HER2, GD2, cMET, EGFR, Mesothelin, VEGFR, alpha-folate receptor, CE7R, IL-3, Cancer-testis antigen (CTA), MART-1 gp100, TNF-related apoptosis-inducing ligand, or combinations thereof.

In some aspects, the payload targeting a tumor antigen comprises a peptide, an antibody or an antigen-binding fragment thereof, a chemical compound, an RNA aptamer, or any combination thereof that targets or antagonizes the tumor antigen. In some aspects, the payload targeting a tumor antigen comprises a microprotein, a designed ankyrin repeat protein (darpin), an anticalin, an adnectin, an aptamer, a peptide mimetic molecule, a natural ligand for a receptor, a camelid nanobody, or any combination thereof.

In some aspects, the payload targeting a tumor antigen comprises a full-length antibody, a single domain antibody, a heavy chain only antibody (VHH), a single chain antibody, a shark heavy chain only antibody (VNAR), an scFv, a Fv, a Fab, a Fab′, a F(ab′)2, or any combination thereof.

II.E. Anti-Phagocytic Signal

Clearance of administered EVs by the body's immune system can reduce the efficacy of an administered EV therapy. In some aspects, the surface of the EV is modified to limit or block uptake of the EV by cells of the immune system, e.g., macrophages. In some aspects, the surface of the EV is modified to display one or more surface antigen that inhibits uptake of the EV by a macrophage. In some aspects, the surface antigen is associated with the exterior surface of the EV.

Surface antigens useful in the present disclosure include, but are not limited to, antigens that label a cell as a “self” cell. In some aspects, the surface antigen comprises an anti-phagocytic signal. In some aspects, the anti-phagocytic signal is selected from CD47, CD24, a fragment thereof, and any combination thereof. In certain aspects, the anti-phagocytic signal comprises CD24, e.g., human CD24. In some aspects, the anti-phagocytic signal comprises a fragment of CD24, e.g., human CD24. In certain aspects, the EV is modified to display CD47 or a fragment thereof on the exterior surface of the EV.

CD47, also referred to as leukocyte surface antigen CD47 and integrin associated protein (TAP), as used herein, is a transmembrane protein that is found on many cells in the body. CD47 is often referred to as the “don't eat me” signal, as it signals to immune cells, in particular myeloid cells, that a particular cell expressing and/or displaying CD47 is not a foreign cell. CD47 is the receptor for SIRPA, binding to which prevents maturation of immature dendritic cells and inhibits cytokine production by mature dendritic cells. Interaction of CD47 with SIRPG mediates cell-cell adhesion, enhances superantigen-dependent T-cell-mediated proliferation and costimulates T-cell activation. CD47 is also known to have a role in both cell adhesion by acting as an adhesion receptor for THBS1 on platelets, and in the modulation of integrins. CD47 also plays an important role in memory formation and synaptic plasticity in the hippocampus (by similarity). In addition, CD47 can play a role in membrane transport and/or integrin dependent signal transduction, prevent premature elimination of red blood cells, and be involved in membrane permeability changes induced following virus infection.

In some aspects, an EV disclosed herein is surface-loaded with a human CD47 on the surface of the EV. The canonical amino acid sequence for human CD47 and various known isoforms are shown in Table 4 (UniProtKB—Q08722; SEQ ID NOs: 156-159). In some aspects, the EV is surface-loaded with a polypeptide comprising the amino acid sequence set forth in SEQ ID NO: 156 or a fragment thereof. In some aspects, the EV is surface-loaded with a polypeptide comprising the amino acid sequence set forth in SEQ ID NO: 157 or a fragment thereof. In some aspects, the EV is surface-loaded with a polypeptide comprising the amino acid sequence set forth in SEQ ID NO: 158 or a fragment thereof. In some aspects, the EV is surface-loaded with a polypeptide comprising the amino acid sequence set forth in SEQ ID NO: 159 or a fragment thereof.

TABLE 4
Human CD47 Amino Acid Sequences

| | |
|---|---|
| Canonical | MWPLVAALLLGSACCGSAQLLFNKTKSVEFTFCNDTVVIPCFVTNMEAQN |
| CD47 | TTEVYVKWKFKGRDIYTFDGALNKSTVPTDFSSAKIEVSQLLKGDASLKM |
| | DKSDAVSHTGNYTCEVTELTREGETIIELKYRVVSWFSPNENILIVIFPI |
| | FAILLFWGQFGIKTLKYRSGGMDEKTIALLVAGLVITVIVIVGAILFVPG |
| | EYSLKNATGLGLIVTSTGILILLHYYVFSTAIGLTSFVIAILVIQVIAYI |
| | LAVVGLSLCIAACIPMHGPLLISGLSILALAQLLGLVYMKFVASNQKTIQ |
| | PPRKAVEEPLNAFKESKGMMNDE (SEQ ID NO: 156) |
| CD47 | MWPLVAALLLGSACCGSAQLLFNKTKSVEFTFCNDTVVIPCFVTNMEAQN |
| HUMAN | TTEVYVKWKFKGRDIYTFDGALNKSTVPTDFSSAKIEVSQLLKGDASLKM |
| Isoform | DKSDAVSHTGNYTCEVTELTREGETIIELKYRVVSWFSPNENILIVIFPI |
| OA3-293 | FAILLFWGQFGIKTLKYRSGGMDEKTIALLVAGLVITVIVIVGAILFVPG |
| | EYSLKNATGLGLIVTSTGILILLHYYVFSTAIGLTSFVIAILVIQVIAYI |
| | LAVVGLSLCIAACIPMHGPLLISGLSILALAQLLGLVYMKFV (SEQ ID |
| | NO: 157) |
| CD47 | MWPLVAALLLGSACCGSAQLLFNKTKSVEFTFCNDTVVIPCFVTNMEAQN |
| HUMAN | TTEVYVKWKFKGRDIYTFDGALNKSTVPTDFSSAKIEVSQLLKGDASLKM |
| Isoform | DKSDAVSHTGNYTCEVTELTREGETIIELKYRVVSWFSPNENILIVIFPI |
| OA3-305 | FAILLFWGQFGIKTLKYRSGGMDEKTIALLVAGLVITVIVIVGAILFVPG |
| | EYSLKNATGLGLIVTSTGILILLHYYVFSTAIGLTSFVIAILVIQVIAYI |
| | LAVVGLSLCIAACIPMHGPLLISGLSILALAQLLGLVYMKFVASNQKTIQ |
| | PPRNN (SEQ ID NO: 158) |
| CD47 | MWPLVAALLLGSACCGSAQLLFNKTKSVEFTFCNDTVVIPCFVTNMEAQN |
| HUMAN | TTEVYVKWKFKGRDIYTFDGALNKSTVPTDFSSAKIEVSQLLKGDASLKM |
| Isoform | DKSDAVSHTGNYTCEVTELTREGETIIELKYRVVSWFSPNENILIVIFPI |
| OA3-312 | FAILLFWGQFGIKTLKYRSGGMDEKTIALLVAGLVITVIVIVGAILFVPG |
| | EYSLKNATGLGLIVTSTGILILLHYYVFSTAIGLTSFVIAILVIQVIAYI |
| | LAVVGLSLCIAACIPMHGPLLISGLSILALAQLLGLVYMKFVASNQKTIQ |
| | PPRKAVEEPLN (SEQ ID NO: 159) |

In some aspects, the EV is surface-loaded with a full length CD47. In some aspects, the EV is surface-loaded with a fragment of CD47, wherein the fragment comprises the extracellular domain of CD47, e.g., human CD47. Any fragment of CD47 that retains an ability to block and/or inhibit phagocytosis by a macrophage can be used in the EVs disclosed herein. In some aspects, the fragment comprises amino acids 19 to about 141 of the canonical human CD47 sequence (e.g., amino acids 19-141 of SEQ ID NO 156). In some aspects, the fragment comprises amino acids 19 to about 135 of the canonical human CD47 sequence (e.g., amino acids 19-135 of SEQ ID NO 156). In some aspects, the fragment comprises amino acids 19 to about 130 of the canonical human CD47 sequence (e.g., amino acids 19-130 of SEQ ID NO 156). In some aspects, the fragment comprises amino acids 19 to about 125 of the canonical human CD47 sequence (e.g., amino acids 19-125 of SEQ ID NO 156).

In some aspects, the EV is surface-loaded with a polypeptide having at least about 70%, at least about 75%, at least about 80%, at least about 85%, at least about 90%, at least about 95%, at least about 96%, at least about 97%, at least about 98%, or at least about 99% sequence identity to amino acids 19 to about 141 of the canonical human CD47 sequence (e.g., amino acids 19-141 of SEQ ID NO 156). In some aspects, the EV is surface-loaded with a polypeptide having at least about 70%, at least about 75%, at least about 80%, at least about 85%, at least about 90%, at least about 95%, at least about 96%, at least about 97%, at least about 98%, or at least about 99% sequence identity to amino acids 19 to about 135 of the canonical human CD47 sequence (e.g., amino acids 19-135 of SEQ ID NO 156). In some aspects, the EV is surface-loaded with a polypeptide having at least about 70%, at least about 75%, at least about 80%, at least about 85%, at least about 90%, at least about 95%, at least about 96%, at least about 97%, at least about 98%, or at least about 99% sequence identity to amino acids 19 to about 130 of the canonical human CD47 sequence (e.g., amino acids 19-130 of SEQ ID NO 156). In some aspects, the EV is surface-loaded with a polypeptide having at least about 70%, at least about 75%, at least about 80%, at least about 85%, at least about 90%, at least about 95%, at least about 96%, at least about 97%, at least about 98%, or at least about 99% sequence identity to amino acids 19 to about 125 of the canonical human CD47 sequence (e.g., amino acids 19-125 of SEQ ID NO 156).

In some aspects, the CD47 or the fragment thereof is modified to increase the affinity of CD47 and its ligand SIRPα. In some aspects, the fragment of CD47 comprises a Velcro-CD47 (see, e.g., Ho et al., JBC 290:12650-63 (2015), which is incorporated by reference herein in its entirety). In some aspects, the Velcro-CD47 comprises a C15S substitution relative to the wild-type human CD47 sequence (SEQ ID NO: 156).

In some aspects, the EV comprises a CD47 or a fragment thereof expressed on the surface of the EV at a level that is higher than an unmodified EV. In some aspects, the CD47 or the fragment thereof is fused with a scaffold protein. Any scaffold protein disclosed herein can be used to express the CD47 or the fragment thereof on the surface of the EV. In some aspects, the EV is modified to express a fragment of CD47 fused to the N-terminus of a Scaffold X protein. In some aspects, the EV is modified to express a fragment of CD47 fused to the N-terminus of PTGFRN.

In some aspects, the EV comprises at least about 20 molecules, at least about 30 molecules, at least about 40, at least about 50, at least about 75, at least about 100, at least about 125, at least about 150, at least about 200, at least about 250, at least about 300, at least about 350, at least about 400, at least about 450, at least about 500, at least about 750, or at least about 1000 molecules of CD47 on the surface of the EV. In some aspects, the EV comprises at least about 20 molecules of CD47 on the surface of the EV. In some aspects, the EV comprises at least about 30 molecules of CD47 on the surface of the EV. In some aspects, the EV comprises at least about 40 molecules of CD47 on the surface of the EV. In some aspects, the EV comprises at least about 50 molecules of CD47 on the surface of the EV. In some aspects, the EV comprises at least about 100 molecules of CD47 on the surface of the EV. In some aspects, the EV comprises at least about 200 molecules of CD47 on the surface of the EV. In some aspects, the EV comprises at least about 300 molecules of CD47 on the surface of the EV. In some aspects, the EV comprises at least about 400 molecules of CD47 on the surface of the EV. In some aspects, the EV comprises at least about 500 molecules of CD47 on the surface of the EV. In some aspects, the EV comprises at least about 1000 molecules of CD47 on the surface of the EV.

In some aspects, CD47 or a fragment thereof on the surface of the EV results in decreased uptake of the EV by myeloid cells as compared to an EV not comprising CD47 or a fragment thereof. In some aspects, uptake by myeloid cells of the EV comprising surface-loaded CD47 or a fragment thereof is decreased by at least about 5%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, or at least about 95%, relative to uptake by myeloid cells of EVs that do not comprise surface-loaded CD47 or a fragment thereof.

In some aspects, CD47 or a fragment thereof on the surface of the EV results in decreased localization of the EV to the liver, as compared to an EV not comprising surface-loaded CD47 or a fragment thereof. In some aspects, localization to the liver of EVs comprising surface-loaded CD47 or a fragment thereof is decreased by at least about 5%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, or at least about 95%, relative to the localization to the liver of EVs not comprising surface-loaded CD47 or a fragment thereof.

In some aspects, the in vivo half-life of an EV comprising surface-loaded CD47 or a fragment thereof is increased relative to the in vivo half-life of an EV that does not comprise surface-loaded CD47 or a fragment thereof. In some aspects, the in vivo half-life of an EV comprising surface-loaded CD47 or a fragment thereof is increased by at least about 1.5-fold, at least about 2-fold, at least about 2.5-fold, at least about 3-fold, at least about 3.5-fold, at least about 4-fold, at least about 4.5-fold, at least about 5-fold, at least about 6-fold, at least about 7-fold, at least about 8-fold, at least about 9-fold, or at least about 10-fold, relative to the in vivo half-life of an EV that does not comprise surface-loaded CD47 or a fragment thereof.

In some aspects, an EV comprising surface-loaded CD47 or a fragment thereof has an increased retention in circulation, e.g., plasma, relative to the retention of an EV that does not comprise surface-loaded CD47 or a fragment thereof in circulation, e.g., plasma. In some aspects, retention in circulation, e.g., plasma, of an EV comprising surface-loaded CD47 or a fragment thereof is increased by at least about 1.5-fold, at least about 2-fold, at least about 2.5-fold, at least about 3-fold, at least about 3.5-fold, at least about 4-fold, at least about 4.5-fold, at least about 5-fold, at least about 6-fold, at least about 7-fold, at least about 8-fold, at least about 9-fold, or at least about 10-fold, relative to the retention in circulation, e.g., plasma, of an EV that does not comprise surface-loaded CD47 or a fragment thereof.

In some aspects, an EV comprising surface-loaded CD47 or a fragment thereof has an altered biodistribution when compared with an exosome that does not comprise surface-loaded CD47 or a fragment. In some aspects, the altered biodistribution leads to increased uptake into endothelial cells, T cells, or increased accumulation in various tissues, including, but not limited to skeletal muscle, cardiac muscle, diaphragm, kidney, bone marrow, central nervous system, lungs, cerebral spinal fluid (CSF), or any combination thereof.

II.F. Scaffold X-Engineered EVss

In some aspects, EVs of the present disclosure comprise a membrane modified in its composition. For example, their membrane compositions can be modified by changing the protein, lipid, or glycan content of the membrane. In certain aspects, the EV is modified by expressing a vNAR, as disclosed herein, fused to a Scaffold X protein.

In some aspects, the surface-engineered EVs are generated by chemical and/or physical methods, such as PEG-induced fusion and/or ultrasonic fusion. In other aspects, the surface-engineered EVs are generated by genetic engineering. EVs produced from a genetically-modified producer cell or a progeny of the genetically-modified cell can contain modified membrane compositions. In some aspects, surface-engineered EVs have scaffold moiety (e.g., exosome protein, e.g., Scaffold X) at a higher or lower density (e.g., higher number) or include a variant or a fragment of the scaffold moiety. In certain aspects, surface-engineered EVs can comprise multiple (e.g., two or more) scaffold moieties on their exterior surface. In some aspects, each of the multiple scaffold moieties are the same. In other aspects, one or more of the multiple scaffold moieties are different.

For example, surface (e.g., Scaffold X)-engineered EVs, can be produced from a cell (e.g., HEK293 cells) transformed with an exogenous sequence encoding a scaffold moiety (e.g., exosome proteins, e.g., Scaffold X) or a variant or a fragment thereof. EVs including scaffold moiety expressed from the exogenous sequence can include modified membrane compositions.

Various modifications or fragments of the scaffold moiety can be used for the aspects of the present disclosure. For example, one or more scaffold moieties modified to have enhanced affinity to a binding agent can be used for generating surface-engineered EV that can be purified using the binding agent. Scaffold moieties modified to be more effectively targeted to EVs and/or membranes can be used. Scaffold moieties modified to comprise a minimal fragment required for specific and effective targeting to exosome membranes can be also used.

Scaffold moieties can be engineered to be expressed as a fusion molecule, e.g., fusion molecule of Scaffold X to one or more exogenous biologically active molecules (e.g., those disclosed herein, e.g., a vNAR that specifically binds TfR, a therapeutic molecule (e.g., an antigen), an adjuvant, and/or an immune modulator). For example, the fusion molecule can comprise a scaffold moiety disclosed herein (e.g., Scaffold X, e.g., PTGFRN, BSG, IGSF2, IGSF3, IGSF8, ITGB1, ITGA4, SLC3A2, ATP transporter, or a fragment or a variant thereof) linked to a vNAR that specifically binds TfR, a therapeutic molecule (e.g., antigen), an adjuvant, and/or an immune modulator. In case of the fusion molecule, the vNAR that specifically binds TfR, therapeutic molecule, adjuvant, and/or immune modulator can be a natural peptide, a recombinant peptide, a synthetic peptide, or any combination thereof.

In some aspects, the surface (e.g., Scaffold X)-engineered EVs described herein demonstrate superior characteristics compared to EVs known in the art. For example, surface (e.g., Scaffold X)-engineered EVs contain modified proteins more highly enriched on their surface than naturally occurring EVs or the EVs produced using conventional exosome proteins. In some aspects, surface (e.g., Scaffold X)-engineered EVs described herein can express greater number (e.g., 2, 3, 4, 5 or more) of exogenous biologically active molecules, such that multiple EVs are not required. Moreover, the surface (e.g., Scaffold X)-engineered EVs of the present disclosure can have greater, more specific, or more controlled biological activity compared to naturally occurring EVs or the EVs produced using conventional exosome proteins.

In some aspects, the Scaffold X comprises Prostaglandin F2 receptor negative regulator (the PTGFRN polypeptide). The PTGFRN protein can be also referred to as CD9 partner 1 (CD9P-1), Glu-Trp-Ile EWI motif-containing protein F (EWI-F), Prostaglandin F2-alpha receptor regulatory protein, Prostaglandin F2-alpha receptor-associated protein, or CD315. The full-length amino acid sequence of the human PTGFRN protein (Uniprot Accession No. Q9P2B2) is shown at Table 1 as SEQ ID NO: 1. The PTGFRN polypeptide contains a signal peptide (amino acids 1 to 25 of SEQ ID NO: 1), the extracellular domain (amino acids 26 to 832 of SEQ ID NO: 1), a transmembrane domain (amino acids 833 to 853 of SEQ ID NO: 1), and a cytoplasmic domain (amino acids 854 to 879 of SEQ ID NO: 1). The mature PTGFRN polypeptide consists of SEQ ID NO: 1 without the signal peptide, i.e., amino acids 26 to 879 of SEQ ID NO: 1. In some aspects, a PTGFRN polypeptide fragment useful for the present disclosure comprises a transmembrane domain of the PTGFRN polypeptide. In other aspects, a PTGFRN polypeptide fragment useful for the present disclosure comprises the transmembrane domain of the PTGFRN polypeptide and (i) at least five, at least 10, at least 15, at least 20, at least 25, at least 30, at least 40, at least 50, at least 70, at least 80, at least 90, at least 100, at least 110, at least 120, at least 130, at least 140, at least 150 amino acids at the N terminus of the transmembrane domain, (ii) at least five, at least 10, at least 15, at least 20, or at least 25 amino acids at the C terminus of the transmembrane domain, or both (i) and (ii).

In some aspects, the fragments of PTGFRN polypeptide lack one or more functional or structural domains, such as IgV.

In other aspects, the Scaffold X comprises an amino acid sequence at least about 70%, at least about 75%, at least about 80%, at least about 85%, at least about 90%, at least about 95%, at least about 96%, at least about 97%, at least about 98%, at least about 99%, or about 100% identical to amino acids 26 to 879 of SEQ ID NO: 1. In other aspects, the Scaffold X comprises an amino acid sequence at least about at least about 70%, at least about 75%, at least about 80%, at least about 85%, at least about 90%, at least about 95%, at least about 96%, at least about 97%, at least about 98%, at least about 99%, or about 100% identical to SEQ ID NO: 33. In other aspects, the Scaffold X comprises the amino acid sequence of SEQ ID NO: 33, except one amino acid mutation, two amino acid mutations, three amino acid mutations, four amino acid mutations, five amino acid mutations, six amino acid mutations, or seven amino acid mutations. The mutations can be a substitution, an insertion, a deletion, or any combination thereof. In some aspects, the Scaffold X comprises the amino acid sequence of SEQ ID NO: 33 and 1 amino acid, two amino acids, three amino acids, four amino acids, five amino acids, six amino acids, seven amino acids, eight amino acids, nine amino acids, ten amino acids, 11 amino acids, 12 amino acids, 13 amino acids, 14 amino acids, 15 amino acids, 16 amino acids, 17 amino acids, 18 amino acids, 19 amino acids, or 20 amino acids or longer at the N terminus and/or C terminus of SEQ ID NO: 33.

TABLE 1
Exemplary Scaffold X Protein Sequences

| Protein | Sequence |
|---|---|
| The | MGRLASRPLLLALLSLALCRGRVVRVPTATLVRVVGTELVIPCNVSDYDGPSEQ |
| PTGFRN | NFDWSFSSLGSSFVELASTWEVGFPAQLYQERLQRGEILLRRTANDAVELHIKN |
| Protein | VQPSDQGHYKCSTPSTDATVQGNYEDTVQVKVLADSLHVGPSARPPPSLSLREG |
| (SEQ ID | EPFELRCTAASASPLHTHLALLWEVHRGPARRSVLALTHEGRFHPGLGYEQRYH |
| NO: 1) | SGDVRLDTVGSDAYRLSVSRALSADQGSYRCIVSEWIAEQGNWQEIQEKAVEVA |
| | TVVIQPSVLRAAVPKNVSVAEGKELDLTCNITTDRADDVRPEVTWSFSRMPDST |
| | LPGSRVLARLDRDSLVHSSPHVALSHVDARSYHLLVRDVSKENSGYYYCHVSLW |
| | APGHNRSWHKVAEAVSSPAGVGVTWLEPDYQVYLNASKVPGFADDPTELACRVV |
| | DTKSGEANVRFTVSWYYRMNRRSDNVVTSELLAVMDGDWTLKYGERSKQRAQDG |
| | DFIFSKEHTDTFNFRIQRTTEEDRGNYYCVVSAWTKQRNNSWVKSKDVFSKPVN |
| | IFWALEDSVLVVKARQPKPFFAAGNTFEMTCKVSSKNIKSPRYSVLIMAEKPVG |
| | DLSSPNETKYIISLDQDSVVKLENWTDASRVDGVVLEKVQEDEFRYRMYQTQVS |
| | DAGLYRCMVTAWSPVRGSLWREAATSLSNPIEIDFQTSGPIFNASVHSDTPSVI |
| | RGDLIKLFCIITVEGAALDPDDMAFDVSWFAVHSFGLDKAPVLLSSLDRKGIVT |
| | TSRRDWKSDLSLERVSVLEFLLQVHGSEDQDFGNYYCSVTPWVKSPTGSWQKEA |
| | EIHSKPVFITVKMDVLNAFKYPLLIGVGLSTVIGLLSCLIGYCSSHWCCKKEVQ |
| | ETRRERRRLMSMEMD |
| The | GPIFNASVHSDTPSVIRGDLIKLFCIITVEGAALDPDDMAFDVSWFAVHSFGLD |
| PTGFRN | KAPVLLSSLDRKGIVTTSRRDWKSDLSLERVSVLEFLLQVHGSEDQDFGNYYCS |
| protein | VTPWVKSPTGSWQKEAEIHSKPVFITVKMDVLNAFKYPLLIGVGLSTVIGLLSC |
| Fragment | LIGYCSSHWCCKKEVQETRRERRRLMSMEM 687-878 of SEQ ID NO: 1 |
| (SEQ ID | |
| NO: 33) | |
| The BSG | MAAALFVLLG FALLGTHGAS GAAGFVQAPL SQQRWVGGSV ELHCEAVGSP |
| protein | VPEIQWWFEG QGPNDTCSQL WDGARLDRVH IHATYHQHAA STISIDTLVE |
| (SEQ ID | EDTGTYECRA SNDPDRNHLT RAPRVKWVRA QAVVLVLEPG TVFTTVEDLG |
| NO: 9) | SKILLTCSLN DSATEVTGHR WLKGGVVLKE DALPGQKTEF KVDSDDQWGE |
| | YSCVFLPEPM GTANIQLHGP PRVKAVKSSE HINEGETAML VCKSESVPPV |
| | TDWAWYKITD SEDKALMNGS ESRFFVSSSQ GRSELHIENL NMEADPGQYR |
| | CNGTSSKGSD QAIITLRVRS HLAALWPFLG IVAEVLVLVT IIFIYEKRRK |
| | PEDVLDDDDA GSAPLKSSGQ HQNDKGKNVR QRNSS |
| The IGSF8 | MGALRPTLLP PSLPLLLLLM LGMGCWAREV LVPEGPLYRV AGTAVSISCN |
| protein | VTGYEGPAQQ NFEWFLYRPE APDTALGIVS TKDTQFSYAV FKSRVVAGEV |
| (SEQ ID | QVQRLQGDAV VLKIARLQAQ DAGIYECHTP STDTRYLGSY SGKVELRVLP |
| NO: 14) | DVLQVSAAPP GPRGRQAPTS PPRMTVHEGQ ELALGCLART STQKHTHLAV |
| | SFGRSVPEAP VGRSTLQEVV GIRSDLAVEA GAPYAERLAA GELRLGKEGT |
| | DRYRMVVGGA QAGDAGTYHC TAAEWIQDPD GSWAQIAEKR AVLAHVDVQT |
| | LSSQLAVTVG PGERRIGPGE PLELLCNVSG ALPPAGRHAA YSVGWEMAPA |
| | GAPGPGRLVA QLDTEGVGSL GPGYEGRHIA MEKVASRTYR LRLEAARPGD |
| | AGTYRCLAKA YVRGSGTRLR EAASARSRPL PVHVREEGVV LEAVAWLAGG |
| | TVYRGETASL LCNISVRGGP PGLRLAASWW VERPEDGELS SVPAQLVGGV |
| | GQDGVAELGV RPGGGPVSVE LVGPRSHRLR LHSLGPEDEG VYHCAPSAWV |
| | QHADYSWYQA GSARSGPVTV YPYMHALDTL FVPLLVGTGV ALVTGATVLG |
| | TITCCFMKRL RKR |
| The ITGB1 | MNLQPIFWIG LISSVCCVFA QTDENRCLKA NAKSCGECIQ AGPNCGWCTN |
| protein | STFLQEGMPT SARCDDLEAL KKKGCPPDDI ENPRGSKDIK KNKNVTNRSK |
| (SEQ ID | GTAEKLKPED ITQIQPQQLV LRLRSGEPQT FTLKFKRAED YPIDLYYLMD |
| NO: 21) | LSYSMKDDLE NVKSLGTDLM NEMRRITSDF RIGFGSFVEK TVMPYISTTP |
| | AKLRNPCTSE QNCTSPFSYK NVLSLTNKGE VFNELVGKQR ISGNLDSPEG |
| | GFDAIMQVAV CGSLIGWRNV TRLLVFSTDA GFHFAGDGKL GGIVLPNDGQ |
| | CHLENNMYTM SHYYDYPSIA HLVQKLSENN IQTIFAVTEE FQPVYKELKN |
| | LIPKSAVGTL SANSSNVIQL IIDAYNSLSS EVILENGKLS EGVTISYKSY |
| | CKNGVNGTGE NGRKCSNISI GDEVQFEISI TSNKCPKKDS DSFKIRPLGF |
| | TEEVEVILQY ICECECQSEG IPESPKCHEG NGTFECGACR CNEGRVGRHC |
| | ECSTDEVNSE DMDAYCRKEN SSEICSNNGE CVCGQCVCRK RDNTNEIYSG |
| | ASNGQICNGR GICECGVCKC TDPKFQGQTC EMCQTCLGVC AEHKECVQCR |
| | AFNKGEKKDT CTQECSYFNI TKVESRDKLP QPVQPDPVSH CKEKDVDDCW |
| | FYFTYSVNGN NEVMVHVVEN PECPTGPDII PIVAGVVAGI VLIGLALLLI |
| | WKLLMIIHDR REFAKFEKEK MNAKWDTGEN PIYKSAVTTV VNPKYEGK |
| The ITGA4 | MAWEARREPG PRRAAVRETV MLLLCLGVPT GRPYNVDTES ALLYQGPHNT |
| protein | LFGYSVVLHS HGANRWLLVG APTANWLANA SVINPGAIYR CRIGKNPGQT |
| (SEQ ID | CEQLQLGSPN GEPCGKTCLE ERDNQWLGVT LSRQPGENGS IVTCGHRWKN |
| NO: 22) | IFYIKNENKL PTGGCYGVPP DLRTELSKRI APCYQDYVKK FGENFASCQA |
| | GISSFYTKDL IVMGAPGSSY WTGSLFVYNI TTNKYKAFLD KQNQVKFGSY |
| | LGYSVGAGHF RSQHTTEVVG GAPQHEQIGK AYIFSIDEKE LNILHEMKGK |
| | KLGSYFGASV CAVDLNADGF SDLLVGAPMQ STIREEGRVF VYINSGSGAV |
| | MNAMETNLVG SDKYAARFGE SIVNLGDIDN DGFEDVAIGA PQEDDLQGAI |
| | YIYNGRADGI SSTFSQRIEG LQISKSLSMF GQSISGQIDA DNNGYVDVAV |
| | GAFRSDSAVL LRTRPVVIVD ASLSHPESVN RTKFDCVENG WPSVCIDLTL |
| | CFSYKGKEVP GYIVLFYNMS LDVNRKAESP PRFYFSSNGT SDVITGSIQV |
| | SSREANCRTH QAFMRKDVRD ILTPIQIEAA YHLGPHVISK RSTEEFPPLQ |
| | PILQQKKEKD IMKKTINFAR FCAHENCSAD LQVSAKIGFL KPHENKTYLA |
| | VGSMKTLMLN VSLFNAGDDA YETTLHVKLP VGLYFIKILE LEEKQINCEV |
| | TDNSGVVQLD CSIGYIYVDH LSRIDISFLL DVSSLSRAEE DLSITVHATC |
| | ENEEEMDNLK HSRVTVAIPL KYEVKLTVHG FVNPTSFVYG SNDENEPETC |
| | MVEKMNLTFH VINTGNSMAP NVSVEIMVPN SFSPQTDKLF NILDVQTTTG |
| | ECHFENYQRV CALEQQKSAM QTLKGIVRFL SKTDKRLLYC IKADPHCLNF |
| | LCNFGKMESG KEASVHIQLE GRPSILEMDE TSALKFEIRA TGFPEPNPRV |
| | IELNKDENVA HVLLEGLHHQ RPKRYFTIVI ISSSLLLGLI VLLLISYVMW |
| | KAGFFKRQYK SILQEENRRD SWSYINSKSN DD |
| The | MELQPPEASI AVVSIPRQLP GSHSEAGVQG LSAGDDSELG SHCVAQTGLE |
| SLC3A2 | LLASGDPLPS ASQNAEMIET GSDCVTQAGL QLLASSDPPA LASKNAEVTG |
| Protein, | TMSQDTEVDM KEVELNELEP EKQPMNAASG AAMSLAGAEK NGLVKIKVAE |
| where | DEAEAAAAAK FTGLSKEELL KVAGSPGWVR TRWALLLLFW LGWLGMLAGA |
| the first | VVIIVRAPRC RELPAQKWWH TGALYRIGDL QAFQGHGAGN LAGLKGRLDY |
| Metis | LSSLKVKGLV LGPIHKNQKD DVAQTDLLQI DPNFGSKEDF DSLLQSAKKK |
| processed. | SIRVILDLTP NYRGENSWFS TQVDTVATKV KDALEFWLQA GVDGFQVRDI |
| (SEQ ID | ENLKDASSFL AEWQNITKGF SEDRLLIAGT NSSDLQQILS LLESNKDLLL |
| NO: 23) | TSSYLSDSGS TGEHTKSLVT QYLNATGNRW CSWSLSQARL LTSFLPAQLL |
| | RLYQLMLFTL PGTPVFSYGD EIGLDAAALP GQPMEAPVML WDESSFPDIP |
| | GAVSANMTVK GQSEDPGSLL SLFRRLSDQR SKERSLLHGD FHAFSAGPGL |
| | FSYIRHWDQN ERFLVVLNFG DVGLSAGLQA SDLPASASLP AKADLLLSTQ |
| | PGREEGSPLE LERLKLEPHE GLLLRFPYAA |

Non-limiting examples of other Scaffold X proteins can be found at U.S. Pat. No. 10,195,290B1, issued Feb. 5, 2019, which is incorporated by reference in its entirety.

In some aspects, the sequence encodes a fragment of the scaffold moiety lacking at least 5, 10, 50, 100, 200, 300, 400, 500, 600, 700, or 800 amino acids from the N-terminus of the native protein. In some aspects, the sequence encodes a fragment of the scaffold moiety lacking at least 5, 10, 50, 100, 200, 300, 400, 500, 600, 700, or 800 amino acids from the C-terminus of the native protein. In some aspects, the sequence encodes a fragment of the scaffold moiety lacking at least 5, 10, 50, 100, 200, 300, 400, 500, 600, 700, or 800 amino acids from both the N-terminus and C-terminus of the native protein. In some aspects, the sequence encodes a fragment of the scaffold moiety lacking one or more functional or structural domains of the native protein.

In some aspects, the scaffold moieties, e.g., Scaffold X, e.g., a PTGFRN protein, are linked to one or more heterologous proteins. The one or more heterologous proteins can be linked to the N-terminus of the scaffold moieties. The one or more heterologous proteins can be linked to the C-terminus of the scaffold moieties. In some aspects, the one or more heterologous proteins are linked to both the N-terminus and the C-terminus of the scaffold moieties. In some aspects, the heterologous protein is a mammalian protein. In some aspects, the heterologous protein is a human protein.

In some aspects, Scaffold X can be used to link any moiety to the luminal surface and on the exterior surface of the EV at the same time. For example, the PTGFRN polypeptide can be used to link a therapeutic molecule (e.g., an antigen), an adjuvant, and/or an immune modulator inside the lumen (e.g., on the luminal surface) in addition to the exterior surface of the EV. Therefore, in certain aspects, Scaffold X can be used for dual purposes, e.g., a therapeutic molecule (e.g., an antigen) on the luminal surface and an adjuvant or immune modulator on the exterior surface of the EV a therapeutic molecule (e.g., an antigen) on the exterior surface of the EV and the adjuvant or immune modulator on the luminal surface, an adjuvant on the luminal surface and an immune modulator on the exterior surface of the EV or an immune modulator on the luminal surface and an adjuvant on the exterior surface of the EV.

II.G. Scaffold Y-Engineered EVs

In some aspects, EVs of the present disclosure comprise an internal space (i.e., lumen) that is different from that of the naturally occurring EVs. For example, the EV can be changed such that the composition in the luminal surface of the EV has the protein, lipid, or glycan content different from that of the naturally-occurring exosomes (e.g., comprises multiple exogenous biologically active molecules disclosed herein).

In some aspects, engineered EVs can be produced from a cell transformed with an exogenous sequence encoding a scaffold moiety (e.g., exosome proteins, e.g., Scaffold Y) or a modification or a fragment of the scaffold moiety that changes the composition or content of the luminal surface of the EV. Various modifications or fragments of the exosome protein that can be expressed on the luminal surface of the EV can be used for the aspects of the present disclosure.

In some aspects, a vNAR that specifically binds TfRis fused to a Scaffold X protein, wherein the vNAR is expressed on the surface of the EV and a second therapeutic protein is fused to a Scaffold Y protein. In some aspects, the second therapeutic protein is fused to a Scaffold Y protein within the lumen of the EV.

In some aspects, the exosome proteins that can change the luminal surface of the EVs include, but are not limited to, the myristoylated alanine rich Protein Kinase C substrate (MARCKS) protein, the myristoylated alanine rich Protein Kinase C substrate like 1 (MARCKSL1) protein, the brain acid soluble protein 1 (BASP1) protein, or any combination thereof. In certain aspects, EVs of the present disclosure comprise two or more (e.g., 2, 3, 4, 5 or more) of such exosome proteins.

In some aspects, Scaffold Y comprises the MARCKS protein (Uniprot accession no. P29966). The MARCKS protein is also known as protein kinase C substrate, 80 kDa protein, light chain. The full-length human MARCKS protein is 332 amino acids in length and comprises a calmodulin-binding domain at amino acid residues 152-176. In some aspects, Scaffold Y comprises the MARCKSL1 protein (Uniprot accession no. P49006). The MARCKSL1 protein is also known as MARCKS-like protein 1, and macrophage myristoylated alanine-rich C kinase substrate. The full-length human MARCKSL1 protein is 195 amino acids in length. The MARCKSL1 protein has an effector domain involved in lipid-binding and calmodulin-binding at amino acid residues 87-110. In some aspects, the Scaffold Y comprises the BASP1 protein (Uniprot accession number P80723). The BASP1 protein is also known as 22 kDa neuronal tissue-enriched acidic protein or neuronal axonal membrane protein NAP-22. The full-length human BASP1 protein sequence (isomer 1) is 227 amino acids in length. An isomer produced by an alternative splicing is missing amino acids 88 to 141 from SEQ ID NO: 49 (isomer 1). Table 2 provides the full-length sequences for the exemplary Scaffold Y disclosed herein (i.e., the MARCKS, MARCKSL1, and BASP1 proteins).

TABLE 2
Exemplary Scaffold Y Protein Sequences

| Protein | Sequence |
|---|---|
| The MARCKS | MGAQFSKTAA KGEAAAERPG EAAVASSPSK ANGQENGHVK VNGDASPAAA |
| protein | ESGAKEELQA NGSAPAADKE EPAAAGSGAA SPSAAEKGEP AAAAAPEAGA |
| (SEQ ID NO: | SPVEKEAPAE GEAAEPGSPT AAEGEAASAA SSTSSPKAED GATPSPSNET |
| 47) | PKKKKKRFSF KKSFKLSGFS FKKNKKEAGE GGEAEAPAAE GGKDEAAGGA |
| | AAAAAEAGAA SGEQAAAPGE EAAAGEEGAA GGDPQEAKPQ EAAVAPEKPP |
| | ASDETKAAEE PSKVEEKKAE EAGASAAACE APSAAGPGAP PEQEAAPAEE |
| | PAAAAASSAC AAPSQEAQPE CSPEAPPAEA AE |
| The | MGSQSSKAPR GDVTAEEAAG ASPAKANGQE NGHVKSNGDL SPKGEGESPP |
| MARCKSL1 | VNGTDEAAGA TGDAIEPAPP SQGAEAKGEV PPKETPKKKK KFSFKKPFKL |
| protein | SGLSFKRNRK EGGGDSSASS PTEEBQEQGE IGACSDEGTA QEGKAAATPE |
| (SEQ ID NO: | SQEPQAKGAE ASAASEEEAG PQATEPSTPS GPESGPTPAS AEQNE |
| 48) | |
| The BASP1 | MGGKLSKKKK GYNVNDEKAK EKDKKAEGAA TEEEGTPKES EPQAAAEPAE |
| protein | AKEGKEKPDQ DAEGKAEEKE GEKDAAAAKE EAPKAEPEKT EGAAEAKAEP |
| (SEQ ID NO: | PKAPEQEQAA PGPAAGGEAP KAAEAAAAPA ESAAPAAGEE PSKEEGEPKK |
| 49) | TEAPAAPAAQ ETKSDGAPAS DSKPGSSEAA PSSKETPAAT EAPSSTPKAQ |
| | GPAASAEEPK PVEAPAANSD QTVTVKE |

The mature BASP1 protein sequence is missing the first Met from SEQ ID NO: 49 and thus contains amino acids 2 to 227 of SEQ ID NO: 49. Similarly, the mature MARCKS and MARCKSL1 proteins also lack the first Met from SEQ ID NOs: 47 and 48, respectively. Accordingly, the mature MARCKS protein contains amino acids 2 to 332 of SEQ ID NO: 47. The mature MARCKSL1 protein contains amino acids 2 to 227 of SEQ ID NO: 48.

In other aspects, Scaffold Y useful for the present disclosure comprises an amino acid sequence at least about 70%, at least about 75%, at least about 80%, at least about 85%, at least about 90%, at least about 95%, at least about 96%, at least about 97%, at least about 98%, at least about 99%, or about 100% identical to amino acids 2 to 227 of SEQ ID NO: 49. In other aspects, the Scaffold Y comprises an amino acid sequence at least about at least about 70%, at least about 75%, at least about 80%, at least about 85%, at least about 90%, at least about 95%, at least about 96%, at least about 97%, at least about 98%, at least about 99%, or about 100% identical to any one of SEQ ID NOs: 50-155. In other aspects, a Scaffold Y useful for the present disclosure comprises the amino acid sequence of SEQ ID NO: 49, except one amino acid mutation, two amino acid mutations, three amino acid mutations, four amino acid mutations, five amino acid mutations, six amino acid mutations, or seven amino acid mutations. The mutations can be a substitution, an insertion, a deletion, or any combination thereof. In some aspects, a Scaffold Y useful for the present disclosure comprises the amino acid sequence of any one of SEQ ID NOs: 50-155 and 1 amino acid, two amino acids, three amino acids, four amino acids, five amino acids, six amino acids, seven amino acids, eight amino acids, nine amino acids, ten amino acids, 11 amino acids, 12 amino acids, 13 amino acids, 14 amino acids, 15 amino acids, 16 amino acids, 17 amino acids, 18 amino acids, 19 amino acids, or 20 amino acids or longer at the N terminus and/or C terminus of SEQ ID NOs: 50-155.

In some aspects, the Scaffold Y comprises or consists of any Scaffold sequence disclosed in Int'l Pub. No. WO 2020/101740, which is incorporated by reference herein in its entirety. In certain aspects, the Scaffold Y sequence comprises or consists of a sequence set forth in Table 1 of Int'l Pub. No. WO 2020/101740.

In some aspects, the Scaffold Y protein useful for the present disclosure does not contain an N-terminal Met. In some aspects, the Scaffold Y protein comprises a lipidated amino acid, e.g., a myristoylated amino acid, at the N-terminus of the scaffold protein, which functions as a lipid anchor. In some aspects, the amino acid residue at the N-terminus of the scaffold protein is Gly. The presence of an N-terminal Gly is an absolute requirement for N-myristoylation. In some aspects, the amino acid residue at the N-terminus of the scaffold protein is synthetic. In some aspects, the amino acid residue at the N-terminus of the scaffold protein is a glycine analog, e.g., allylglycine, butylglycine, or propargylglycine.

Non-limiting examples of scaffold proteins can be found at WO/2019/099942, published May 23, 2019 and WO/2020/101740, published May 22, 2020, which are incorporated by reference in their entireties.

In some aspects, the scaffold protein comprises a scaffold protein disclosed in WO/2020/163370, which is incorporated by reference herein in its entirety.

In other aspects, the lipid anchor can be any lipid anchor known in the art, e.g., palmitic acid or glycosylphosphatidylinositols. Under unusual circumstances, e.g., by using a culture medium where myristic acid is limiting, some other fatty acids including shorter-chain and unsaturated, can be attached to the N-terminal glycine. For example, in BK channels, myristate has been reported to be attached posttranslationally to internal serine/threonine or tyrosine residues via a hydroxyester linkage. Membrane anchors known in the art are presented in the following table:

| Modification | Modifying Group |
|---|---|
| S-Palmitoylation | 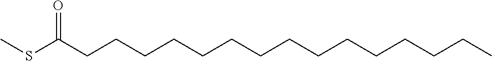 [SMILES: CCCCCCCCCCCCCCCC(=O)SC] |
| N-Palmitoylation | 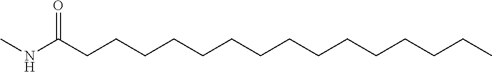 [SMILES: CCCCCCCCCCCCCCCC(=O)NC] |
| N-Myristoylation | 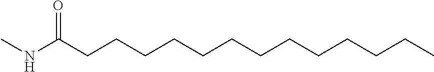 [SMILES: CCCCCCCCCCCCCC(=O)NC] |
| O-Acylation | 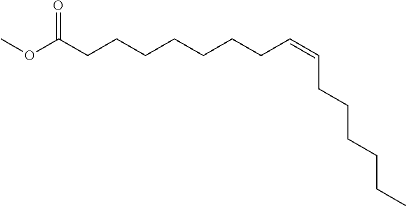 [SMILES: CCCCCC/C=C\CCCCCCCC(=O)OC] |
| Farnesylation | 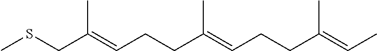 [SMILES: C/C=C(\C)CC/C=C(\C)CC/C=C(\C)CSC] |
| Geranylgeranylation | 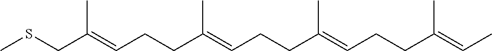 [SMILES: C/C=C(\C)CC/C=C(\C)CC/C=C(\C)CC/C=C(\C)CSC] |
| Cholesterol | 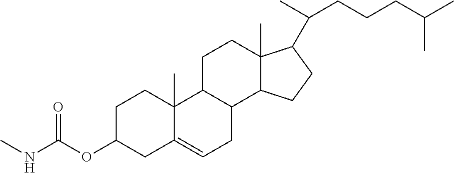 [SMILES: CNC(=O)OC1CCC2(C)C(=CCC3C2CCC2(C)C(C(C)CCCC(C)C)CCC32)C1] |

II.H. Linkers

As described supra, extracellular vesicles (EVs) of the present disclosure (e.g., exosomes and nanovesicles) can comprises one or more linkers that link one or more vNAR and/or exogenous biologically active molecules disclosed herein (e.g., targeting moiety, therapeutic molecule (e.g., antigen), adjuvant, or immune modulator) to the EVs (e.g., to the exterior surface or on the luminal surface). In some aspects, the one or more exogenous biologically active molecules (e.g., targeting moiety, therapeutic molecule, adjuvant, or immune modulator) are linked to the EVs directly or via one or more scaffold moieties (e.g., Scaffold X or Scaffold Y). For example, in certain aspects, one or more exogenous biologically active molecules are linked to the exterior surface of an exosome via Scaffold X. In further aspects, one or more exogenous biologically active molecules are linked to the luminal surface of an exosome via Scaffold X or Scaffold Y. The linker can be any chemical moiety known in the art.

As used herein, the term “linker” refers to a peptide or polypeptide sequence (e.g., a synthetic peptide or polypeptide sequence) or to a non-polypeptide, e.g., an alkyl chain. In some aspects, two or more linkers can be linked in tandem. When multiple linkers are present, each of the linkers can be the same or different. Generally, linkers provide flexibility or prevent/ameliorate steric hindrances. Linkers are not typically cleaved; however, in certain aspects, such cleavage can be desirable. Accordingly, in some aspects, a linker can comprise one or more protease-cleavable sites, which can be located within the sequence of the linker or flanking the linker at either end of the linker sequence.

In some aspects, the linker is a peptide linker. In some aspects, the peptide linker can comprise at least about two, at least about three, at least about four, at least about five, at least about 10, at least about 15, at least about 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, or at least about 100 amino acids.

In some aspects, the peptide linker is synthetic, i.e., non-naturally occurring. In one aspect, a peptide linker includes peptides (or polypeptides) (e.g., natural or non-naturally occurring peptides) which comprise an amino acid sequence that links or genetically fuses a first linear sequence of amino acids to a second linear sequence of amino acids to which it is not naturally linked or genetically fused in nature. For example, in one aspect the peptide linker can comprise non-naturally occurring polypeptides, which are modified forms of naturally occurring polypeptides (e.g., comprising a mutation such as an addition, substitution or deletion).

Linkers can be susceptible to cleavage (“cleavable linker”) thereby facilitating release of the exogenous biologically active molecule (e.g., targeting moiety, therapeutic molecule, adjuvant, or immune modulator).

In some aspects, the linker is a “reduction-sensitive linker.” In some aspects, the reduction-sensitive linker contains a disulfide bond. In some aspects, the linker is an “acid labile linker.” In some aspects, the acid labile linker contains hydrazone. Suitable acid labile linkers also include, for example, a cis-aconitic linker, a hydrazide linker, a thiocarbamoyl linker, or any combination thereof.

In some aspects, the linker comprises a non-cleavable linker.

### III. Pharmaceutical Compositions

Certain aspects of the present disclosure are directed to a pharmaceutical composition comprising an EV disclosed herein. In some aspects, an EV disclosed herein is formulated in a pharmaceutical composition. In some aspects, the pharmaceutical composition comprising the EV further comprises a pharmaceutically acceptable carrier or excipient. Pharmaceutically acceptable excipients or carriers can be determined in part by the particular composition being administered, as well as by the particular method used to administer the composition. Accordingly, there is a wide variety of suitable formulations of compositions (e.g., pharmaceutical compositions) comprising a plurality of extracellular vesicles. (See, e.g., Remington's Pharmaceutical Sciences, Mack Publishing Co., Easton, Pa. 21st ed. (2005)). The pharmaceutical compositions are generally formulated sterile and in full compliance with all Good Manufacturing Practice (GMP) regulations of the U.S. Food and Drug Administration.

In some aspects, a pharmaceutical composition comprises one or more therapeutic agents and an exosome described herein. In certain aspects, the EVs are co-administered with of one or more additional therapeutic agents, in a pharmaceutically acceptable carrier. In some aspects, the pharmaceutical composition comprising the EV is administered prior to administration of the additional therapeutic agents. In other aspects, the pharmaceutical composition comprising the EV is administered after the administration of the additional therapeutic agents. In further aspects, the pharmaceutical composition comprising the EV is administered concurrently with the additional therapeutic agents.

Acceptable carriers, excipients, or stabilizers are nontoxic to recipients (e.g., animals or humans) at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

Examples of carriers or diluents include, but are not limited to, water, saline, Ringer's solutions, dextrose solution, and 5% human serum albumin. The use of such media and compounds for pharmaceutically active substances is well known in the art. Except insofar as any conventional media or compound is incompatible with the extracellular vesicles described herein, use thereof in the compositions is contemplated. Supplementary therapeutic agents can also be incorporated into the compositions. Typically, a pharmaceutical composition is formulated to be compatible with its intended route of administration. The EVs can be administered by parenteral, topical, intravenous, oral, subcutaneous, intra-arterial, intradermal, transdermal, rectal, intracranial, intraperitoneal, intranasal, intratumoral, intramuscular route or as inhalants. In certain aspects, the pharmaceutical composition comprising exosomes is administered intravenously, e.g. by injection. The EVs can optionally be administered in combination with other therapeutic agents that are at least partly effective in treating the disease, disorder or condition for which the EVs are intended.

Solutions or suspensions can include the following components: a sterile diluent such as water, saline solution, fixed oils, polyethylene glycols, glycerine, propylene glycol or other synthetic solvents; antibacterial compounds such as benzyl alcohol or methyl parabens; antioxidants such as ascorbic acid or sodium bisulfite; chelating compounds such as ethylenediaminetetraacetic acid (EDTA); buffers such as acetates, citrates or phosphates, and compounds for the adjustment of tonicity such as sodium chloride or dextrose. The pH can be adjusted with acids or bases, such as hydrochloric acid or sodium hydroxide. The preparation can be enclosed in ampoules, disposable syringes or multiple dose vials made of glass or plastic.

Pharmaceutical compositions suitable for injectable use include sterile aqueous solutions (if water soluble) or dispersions and sterile powders. For intravenous administration, suitable carriers include physiological saline, bacteriostatic water, Cremophor EL™ (BASF, Parsippany, N.J.) or phosphate buffered saline (PBS). The composition is generally sterile and fluid to the extent that easy syringeability exists. The carrier can be a solvent or dispersion medium containing, e.g., water, ethanol, polyol (e.g., glycerol, propylene glycol, and liquid polyethylene glycol, and the like), and suitable mixtures thereof. The proper fluidity can be maintained, e.g., by the use of a coating such as lecithin, by the maintenance of the required particle size in the case of dispersion and by the use of surfactants. Prevention of the action of microorganisms can be achieved by various antibacterial and antifungal compounds, e.g., parabens, chlorobutanol, phenol, ascorbic acid, thimerosal, and the like. If desired, isotonic compounds, e.g., sugars, polyalcohols such as manitol, sorbitol, and sodium chloride can be added to the composition. Prolonged absorption of the injectable compositions can be brought about by including in the composition a compound which delays absorption, e.g., aluminum monostearate and gelatin.

Sterile injectable solutions can be prepared by incorporating the EVs in an effective amount and in an appropriate solvent with one or a combination of ingredients enumerated herein, as desired. Generally, dispersions are prepared by incorporating the EVs into a sterile vehicle that contains a basic dispersion medium and any desired other ingredients. In the case of sterile powders for the preparation of sterile injectable solutions, methods of preparation are vacuum drying and freeze-drying that yields a powder of the active ingredient plus any additional desired ingredient from a previously sterile-filtered solution thereof. The EVs can be administered in the form of a depot injection or implant preparation which can be formulated in such a manner to permit a sustained or pulsatile release of the EV.

In certain aspects, the pharmaceutical composition comprising exosomes is administered as a liquid suspension. In certain aspects, the pharmaceutical composition is administered as a formulation that is capable of forming a depot following administration. In certain preferred aspects, the depot slowly releases the EVs into circulation, or remains in depot form.

Typically, pharmaceutically-acceptable compositions are highly purified to be free of contaminants, are biocompatible and not toxic, and are suited to administration to a subject. If water is a constituent of the carrier, the water is highly purified and processed to be free of contaminants, e.g., endotoxins.

The pharmaceutically-acceptable carrier can be lactose, dextrose, sucrose, sorbitol, mannitol, starch, gum acacia, calcium phosphate, alginates, gelatin, calcium silicate, micro-crystalline cellulose, polyvinylpyrrolidone, cellulose, water, syrup, methyl cellulose, methylhydroxy benzoate, propylhydroxy benzoate, talc, magnesium stearate, and/or mineral oil, but is not limited thereto. The pharmaceutical composition can further include a lubricant, a wetting agent, a sweetener, a flavor enhancer, an emulsifying agent, a suspension agent, and/or a preservative.

The pharmaceutical compositions described herein comprise the EVs described herein and optionally a pharmaceutically active or therapeutic agent. The therapeutic agent can be a biological agent, a small molecule agent, or a nucleic acid agent.

Dosage forms are provided that comprise a pharmaceutical composition comprising the EVs described herein. In some aspects, the dosage form is formulated as a liquid suspension for intravenous injection. In some aspects, the dosage form is formulated as a liquid suspension for intratumoral injection.

In certain aspects, the preparation of exosomes is subjected to radiation, e.g., X rays, gamma rays, beta particles, alpha particles, neutrons, protons, elemental nuclei, UV rays in order to damage residual replication-competent nucleic acids.

In certain aspects, the preparation of exosomes is subjected to gamma irradiation using an irradiation dose of more than 1, 5, 10, 15, 20, 25, 30, 35, 40, 50, 60, 70, 80, 90, 100, or more than 100 kGy.

In certain aspects, the preparation of exosomes is subjected to X-ray irradiation using an irradiation dose of more than 0.1, 0.5, 1, 5, 10, 15, 20, 25, 30, 35, 40, 50, 60, 70, 80, 90, 100, 200, 300, 400, 500, 600, 700, 800, 900, 1000, 2000, 3000, 4000, 5000, 6000, 7000, 8000, 9000, 10000, or greater than 10000 mSv.

### IV. Methods of the Disclosure

Certain aspects of the present disclosure are directed to methods of treating a disease or disorder in a subject in need thereof comprising administering an EV disclosed herein. Some aspects of the present disclosure are directed to methods of targeting an extracellular vesicle (EV) to a cell of the central nervous system, comprising loading a vNAR that specifically binds transferrin receptor (TfR) on the surface of the extracellular vesicle. Some aspects of the present disclosure are directed to methods of increasing the transport of an EV across the blood brain barrier in a human subject, comprising loading a vNAR on the surface of the EV, wherein the vNAR specifically binds TfR.

In some aspects, the EV comprising the vNAR that specifically binds TfR has increased transport across of the blood brain barrier. In some aspects, the vNAR increases the transport of the EV across the blood brain barrier by at least about 10%, at least about 20%, at least about 30%, at least about 40%, at least about 75%, at least about 100%, at least about 150%, at least about 200%, at least about 250%, at least about 300%, at least about 350%, at least about 400%, at least about 450%, at least about 500%, at least about 600%, at least about 700%, at least about 800%, at least about 900%, or at least about 1000%, as compared to a reference EV not comprising a vNAR that specifically binds human TfR. In some aspects, the vNAR increases the transport of the EV across the blood brain barrier by at least about 10%, at least about 20%, at least about 30%, at least about 40%, at least about 75%, at least about 100%, at least about 150%, at least about 200%, at least about 250%, at least about 300%, at least about 350%, at least about 400%, at least about 450%, at least about 500%, at least about 600%, at least about 700%, at least about 800%, at least about 900%, or at least about 1000%, as compared to an EV comprising an IgG antibody or a fragment thereof that specifically binds human TfR. In some aspects, the vNAR increases the transport of the EV across the blood brain barrier by at least about 10%, at least about 20%, at least about 30%, at least about 40%, at least about 75%, at least about 100%, at least about 150%, at least about 200%, at least about 250%, at least about 300%, at least about 350%, at least about 400%, at least about 450%, at least about 500%, at least about 600%, at least about 700%, at least about 800%, at least about 900%, or at least about 1000%, as compared to an EV comprising an scFv that specifically binds human TfR.

In some aspects, the disease or condition comprises a cancer, e.g., a cancer selected from cancers of the lung, ovarian, cervical, endometrial, breast, brain, colon, prostate, gastrointestinal cancer, head and neck cancer, non-small cell lung cancer, cancer of the nervous system, kidney cancer, retina cancer, skin cancer, liver cancer, pancreatic cancer, genital-urinary cancer and bladder cancer, melanoma, leukemia, brain cancer (e.g., glioma, astrocytomas, ependymomas, oligodendrogliomas, and tumors with mixtures of two or more cell types, called mixed gliomas, Acoustic Neuroma (Neurilemmoma, Schwannoma. Neurinoma), Adenoma, Astracytoma, Low-Grade Astrocytoma, giant cell astrocytomas, Mid- and High-Grade Astrocytoma, Recurrent tumors, Brain Stem Glioma, Chordoma, Choroid Plexus Papilloma, CNS Lymphoma (Primary Malignant Lymphoma), Cysts, Dermoid cysts, Epidermoid cysts, Craniopharyngioma, Ependymoma Anaplastic ependymoma, Gangliocytoma (Ganglioneuroma), Ganglioglioma, Glioblastoma Multiforme (GBM), Malignant Astracytoma, Glioma, Hemangioblastoma, Inoperable Brain Tumors, Lymphoma, Medulloblastoma (MDL), Meningioma, Metastatic Brain Tumors, Mixed Glioma, Neurofibromatosis, Oligodendroglioma. Optic Nerve Glioma, Pineal Region Tumors, Pituitary Adenoma, PNET (Primitive Neuroectodermal Tumor), Spinal Tumors, Subependymoma, and Tuberous Sclerosis (Bourneville's Disease), and any combination thereof.

IV.A. Methods of Treating Neurological Disease or Disorder

Certain aspects of the present disclosure are directed to methods of treating a neurological disease or disorder, e.g., a neuroimmunological disorder, in a subject in need thereof. In some aspects, the method comprises administering to the subject a therapeutically effective amount of an EV comprising a vNAR that specifically binds TfR, as disclosed herein. In some aspects, the EV is capable of targeted delivery of a therapeutic agent, e.g., as disclosed herein, to the CNS to treat the neurological disorder. In some aspects, the EV is capable of up-regulating an immune response in the subject, thereby enhancing the subject's immune response against the neuroimmunological disorder. In some aspects, the composition is administered intratumorally or intrathecally to the subject.

As used herein, the term “neuroimmunological disorder” refers to diseases and disorders of either the central or peripheral nervous system. The nervous system represents a privileged immune environment that generally dampens inflammatory responses in the brain spinal cord and nerves. This relative low immunoresponsiveness (anergy) is not only a function of the blood brain barrier but also a feature of the resident myeloid cells of the nervous system (e.g., microglia, meningeal macrophages, perivascular macrophages, and choroid plexus macrophages). These cells generally display immunosuppressive phenotypes and are known to become further “immunosilenced” or anergic in the setting of certain pathologies such as cancer or chronic infections. Accordingly, in some aspects, a neuroimmunological disorder can result from an inability of a subject's immune system to mount an effective immune response against the disorder. In other aspects, a neuroimmunological disorder can result from an aberrant or excessive immune response within the nervous system.

In some aspects, a neuroimmunological disorder that can be treated with the present disclosure comprises a brain tumor or chronic infectious meningitis. In certain aspects, a neuroimmunological disorder is a brain tumor. In some aspects, a neuroimmunological disorder is a chronic infectious meningitis. In certain aspects, a chronic infectious meningitis can be associated with tuberculosis, Lyme disease, fungi, or combinations thereof. In some aspects, a neuroimmunological disorder is associated with neoplastic or infectious lesions within the nervous system compartment.

Also provided herein are methods of preventing metastasis of a brain tumor in a subject. The method comprises administering to the subject a therapeutically effective amount of the compositions disclosed herein, wherein the composition is capable of preventing a brain tumor at one location in the subject from promoting the growth of one or more tumors at another location in the subject. In some aspects, the composition is administered intratumorally or intrathecally in a first tumor in one location, and the composition administered in a first tumor prevents metastasis of one or more tumors at a second location.

In some aspects, administering an EV disclosed herein inhibits and/or reduces growth of a brain tumor in a subject. In some aspects, the growth of a brain tumor (e.g., tumor volume or weight) is reduced by at least about 5%, at least about 10%, at least about 20%, at least about 30%, at least about 40%, at least about 50%, at least about 60%, at least about 70%, at least about 80%, at least about 90%, or about 100% compared to a reference (e.g., tumor volume in a corresponding subject after administration of an EV without the vNAR that specifically binds TfR).

As used herein, the term “brain tumor” refers to an abnormal growth of cells within the brain (e.g., within the meninges). Brain tumors can be categorized as primary or secondary brain tumor. “Primary brain tumor” refers to brain tumors that originate within the brain. “Secondary brain tumor” refers to brain tumors that are the result of cancer cells originating at primary sites outside the brain that have metastasized (i.e., spread) to the brain. Unless specified otherwise, the term brain tumor can refer to both primary and secondary brain tumors.

In some aspects, a brain tumor that can be treated with the present disclosure comprises an acoustic neuroma, choroid plexus carcinoma, craniopharyngioma, embryonal tumor, glioma, medulloblastoma, meningioma, pediatric brain tumor, pineoblastoma, pituitary tumor, or combinations thereof.

In certain aspects, a brain tumor that can be treated with the present disclosure comprises a glioma. As used herein, the term “glioma” refers to a type of tumor that starts in the glial cells of the brain or the spine. In some aspects, a glioma can be classified by specific type of cells with which they share histological features. Accordingly, a glioma that can be treated with EVs disclosed herein can be classified as an ependymoma (ependymal cells), astrocytoma (astrocytes), oligodendroglioma (oligodendrocytes), brainstem glioma (e.g., diffuse intrinsic pontine glioma), optic nerve glioma, mixed glioma, oligoastrocytoma, or any combination thereof. In certain aspects, an astrocytoma comprises glioblastoma multiforme (GBM).

Gliomas disclosed herein can be further categorized according to their grade, which is determined by pathologic evaluation of the tumor. In some aspects, the neuropathological evaluation and diagnostics of brain tumor specimens is performed according to WHO Classification of Tumours of the Central Nervous System. In some aspects, a glioma that can be treated with the present disclosure comprises a low-grade glioma. A “low-grade glioma” [WHO grade II] are well-differentiated (not anaplastic) and tend to exhibit benign tendencies and portend a better prognosis for the patient. However, in some aspects, low-grade gliomas can have a uniform rate of recurrence and increase in grade over time, so should be classified as malignant. In some aspects, a glioma that can be treated comprises a high grade glioma. A “high-grade glioma” [WHO grades III-IV] gliomas are undifferentiated or anaplastic and are malignant and carry a worse prognosis. Of numerous grading systems in use, the most common is the World Health Organization (WHO) grading system for astrocytoma, under which tumors are graded from I (least advanced disease—best prognosis) to IV (most advanced disease—worst prognosis). Non-limiting examples of high-grade gliomas include anaplastic astrocytomas and glioblastoma multiforme.

In some aspects, an EV disclosed herein can be used to treat a glioma grade I, grade II, grade III, grade IV, or combinations thereof, as determined under the WHO grading system. In certain aspects, an EV disclosed herein can be used to treat any type of gliomas.

In some aspects, the glioma treatable by the present methods is a diffuse intrinsic pontine glioma (DIPG), a type of brainstem glioma. Diffuse intrinsic pontine glioma primarily affects children, usually between the ages of 5 and 7. The median survival time with DIPG is under 12 months. Surgery to attempt tumor removal is usually not possible or advisable for DIPG. By their very nature, these tumors invade diffusely throughout the brain stem, growing between normal nerve cells.

In other aspects, the glioma treatable by the present methods is an IDH1 and IDH2-mutated glioma. Patients with glioma carrying mutations in either IDH1 or IDH2 have a relatively favorable survival, compared with patients with glioma with wild-type IDH1/2 genes. In WHO grade III glioma, IDH1/2-mutated glioma have a median prognosis of ˜3.5 years, whereas IDH1/2 wild-type glioma perform poor with a median overall survival of 1.5 years. In glioblastoma, the difference is larger.

In some aspects, a neuroimmunological disorder that can be treated with the present disclosure comprises a neoplastic meningitis. As used herein, “neoplastic meningitis” refers to a tumor which has spread from the original tumor site into the dural and leptomeninges, which are thin tissue membranes covering the brain and spinal cord. In some aspects, connective tissue nerve sheaths that extend from the meninges onto and into nerves can also become involved. Neoplastic meningitis is also known as carcinomatous meningitis, leptomeningeal carcinoma, leptomeningeal carcinomatosis, leptomeningeal metastasis, leptomeningeal disease (LMD), leptomeningeal cancer, meningeal carcinomatosis, and meningeal metastasis. In certain aspects, a neoplastic meningitis is caused by leukemia. In some aspects, a neoplastic meningitis is caused by melanoma, breast, lung, gastrointestinal cancer, or combinations thereof. In certain aspects, a neoplastic meningitis is caused by a glioma.

In some aspects, EVs disclosed herein can be used to target the meningeal lymphatic immune system. The meningeal lymphatic system is a network of conventional lymphatic vessels and associated macrophages located parallel to major dural venous sinuses, dural coverings of cerebral arteries and nerve sheaths. This system is responsible for draining the cerebral spinal fluid (CSF), particulate matter and immune cells to specific peripheral lymph nodes that act as sentinel nodes for the nervous system. These lymph nodes include the submandibular, deep cervical and paraspinal nodes. In certain aspects, EVs disclosed herein can be used to target the macrophages lining the meningeal lymphatics or perivascular regions of the central nervous system. In some aspects, targeting such meningeal and/or perivascular macrophages allows for the regulation of an immune response (e.g., against a brain tumor antigen) within the meninges and the brain.

In some aspects, an EV disclosed herein can re-activate macrophages (e.g., within the nervous system) and/or reverse nervous system anergy. In certain aspects, re-activating macrophages (e.g., within the nervous system) and/or reversing nervous system anergy can help treat a neuroimmunological disorder (e.g., by eradicating neoplastic or infectious lesions within the nervous system).

IV.B. Combination Therapies

In some aspects, a method for treating a disease or disorder disclosed herein can comprise administering an EV comprising a vNAR that specifically binds TfR. In certain aspects, the EV disclosed herein can be used in combination with one or more additional therapeutic agents (e.g., immuno-oncology agents), such that multiple elements of the immune pathway can be targeted. Non-limiting of such combinations include: a STING agonist; a therapy that enhances tumor antigen presentation (e.g., dendritic cell vaccine, GM-CSF secreting cellular vaccines, CpG oligonucleotides, imiquimod); a therapy that inhibits negative immune regulation e.g., by inhibiting CTLA-4 and/or PD1/PD-L1/PD-L2 pathway and/or depleting or blocking Tregs or other immune suppressing cells (e.g., myeloid-derived suppressor cells); a therapy that stimulates positive immune regulation, e.g., with agonists that stimulate the CD-137, OX-40, and/or CD40 or GITR pathway and/or stimulate T cell effector function; a therapy that increases systemically the frequency of anti-tumor T cells; a therapy that depletes or inhibits Tregs, such as Tregs in the tumor, e.g., using an antagonist of CD25 (e.g., daclizumab) or by ex vivo anti-CD25 bead depletion; a therapy that impacts the function of suppressor myeloid cells in the tumor; a therapy that enhances immunogenicity of tumor cells (e.g., anthracyclines); adoptive T cell or NK cell transfer including genetically modified cells, e.g., cells modified by chimeric antigen receptors (CAR-T therapy); a therapy that inhibits a metabolic enzyme such as indoleamine dioxygenase (IDO), dioxygenase, arginase, or nitric oxide synthetase; a therapy that reverses/prevents T cell anergy or exhaustion; a therapy that triggers an innate immune activation and/or inflammation at a tumor site; administration of immune stimulatory cytokines; or blocking of immuno repressive cytokines.

In some aspects, an immuno-oncology agent that can be used in combination with EVs disclosed herein comprises an immune checkpoint inhibitor (i.e., blocks signaling through the particular immune checkpoint pathway). Non-limiting examples of immune checkpoint inhibitors that can be used in the present methods comprise a CTLA-4 antagonist (e.g., anti-CTLA-4 antibody), PD-1 antagonist (e.g., anti-PD-1 antibody, anti-PD-L1 antibody), TIM-3 antagonist (e.g., anti-TIM-3 antibody), or combinations thereof.

In some aspects, an immuno-oncology agent comprises an immune checkpoint activator (i.e., promotes signaling through the particular immune checkpoint pathway). In certain aspects, immune checkpoint activator comprises OX40 agonist (e.g., anti-OX40 antibody), LAG-3 agonist (e.g. anti-LAG-3 antibody), 4-1BB (CD137) agonist (e.g., anti-CD137 antibody), GITR agonist (e.g., anti-GITR antibody), or any combination thereof.

In some aspects, EVs disclosed herein can also be used in combination with one or more additional immunomodulating agents. Such agents can include, for example, chemotherapy drugs, small molecule drugs, or antibodies that stimulate the immune response to a given cancer. In some aspects, the methods described herein are used in combination with a standard of care treatment (e.g., surgery, radiation, and chemotherapy).

In some aspects, a combination of an EV disclosed herein and a second agent discussed herein (e.g., immune checkpoint inhibitor) can be administered concurrently as a single composition in a pharmaceutically acceptable carrier. In other aspects, a combination of an EV and a second agent discussed herein (e.g., immune checkpoint inhibitor) can be administered concurrently as separate compositions. In further aspects, a combination of an EV and a second agent discussed herein (e.g., immune checkpoint inhibitor) can be administered sequentially. In some aspects, an EV is administered prior to the administration of a second agent (e.g., immune checkpoint inhibitor).

In some aspects, EVs disclosed herein are administered to a subject via intrathecal administration. In some aspects, the EVs are administered via an injection into the spinal canal, or into the subarachnoid space so that it reaches the cerebrospinal fluid (CSF). Non-limiting examples of other routes of administration that can be used include intravenously, intratumorally, intranasally, periocularly, intraarterially, or combinations thereof.

IV.C. Routes of Administration

The EVs disclosed herein have the unique ability to target cells and/or tissues that express a target antigen, e.g., TfR. As such, the EVs can be administered by any route and still be capable of localizing to the target cell or tissue. In some aspects, the EVs are administered systemically, e.g., intravenously. In some aspects, the EVs are administered compartmentally, e.g., directly into a target tissue. In some aspects, the EVs are administered intravenously to the circulatory system of the subject. In some aspects, the EVs are infused in a suitable liquid and administered into a vein of the subject.

In some aspects, the EVs are administered intra-arterialy to the circulatory system of the subject. In some aspects, the EVs are infused in a suitable liquid and administered into an artery of the subject.

In some aspects, the EVs are administered to the subject by intrathecal administration. In some aspects, the EVs are administered via an injection into the spinal canal, or into the subarachnoid space so that it reaches the cerebrospinal fluid (CSF).

In some aspects, the EVs are administered intratumorally into one or more tumors of the subject.

In some aspects, the EVs are administered to the subject by intranasal administration. In some aspects, the EVs can be insufflated through the nose in a form of either topical administration or systemic administration. In certain aspects, the EVs are administered as nasal spray.

In some aspects, the EVs are administered to the subject by intraperitoneal administration. In some aspects, the EVs are infused in suitable liquid and injected into the peritoneum of the subject. In some aspects, the intraperitoneal administration results in distribution of the EVs to the lymphatics. In some aspects, the intraperitoneal administration results in distribution of the EVs to the thymus, spleen, and/or bone marrow. In some aspects, the intraperitoneal administration results in distribution of the EVs to one or more lymph nodes. In some aspects, the intraperitoneal administration results in distribution of the EVs to one or more of the cervical lymph node, the inguinal lymph node, the mediastinal lymph node, or the sternal lymph node. In some aspects, the intraperitoneal administration results in distribution of the EVs to the pancreas.

In some aspects, the EVs are administered to the subject by periocular administration. In some aspects, the s are injected into the periocular tissues. Periocular drug administration includes the routes of subconjunctival, anterior sub-Tenon's, posterior sub-Tenon's, and retrobulbar administration.

In some aspects, the EVs are administered intraocularly. Accordingly, the present disclosure provides methods of treating an eye disease or disorder in a subject in need thereof comprising administering an effective amount of a composition comprising an extracellular vesicle (EV) of the present disclosure which comprises a payload (e.g., an AVV) to the subject, wherein the administration of the composition is intraocular.

In some aspects, the intraocular administration is selected from the group consisting of intravitreal administration, intracameral administration, subconjunctival administration, subretinal administration, sub scleral administration, intrachoroidal administration, and any combination thereof. In some aspects, the intraocular administration comprises the injection of the EVs of the present disclosure. In some aspects, the intraocular administration is intravitreal injection.

In some aspects, the intraocular administration comprises the implantation of a delivery device comprising the EVs of the present disclosure. In some aspects, the delivery device is an intraocular delivery device. In some aspects, the intraocular delivery device is biodegradable. In some aspects, the intraocular delivery device is an intravitreal implant or a scleral plug. In some aspects, the delivery device is a sustained release delivery device.

In some aspects, the composition comprising an EV of the present disclosure is pre-treated with intravenous immunoglobulin (IVIg) prior to intraocular administration.

In some aspects, the EV is administered by a route selected from intraperitoneal, inhalation, oral, intramuscular, intrathecal, intracranial, intraocular, intradermal, sub-cutaneous, and any combination thereof. In some aspects, the EV is delivered intratumorally. In some aspects, the EV is administered to the periphery of a tumor. In certain aspects, the EV is delivered intraperitoneally. In certain aspects, the EV is delivered by inhalation. In certain aspects, the composition is delivered orally. In certain aspects, the EV is delivered intramuscularly. In certain aspects, the EV is delivered intrathecally. In certain aspects, the EV is delivered intracranially. In certain aspects, the EV is delivered intraocularly. In certain aspects, the EV is delivered intradermally. In certain aspects, the EV is delivered subcutaneously. In some aspects, the EV is delivered by compartmental administration.

In some aspects, the EVs are administered by intrathecal administration, followed by application of a mechanical convective force to the torso. See, e.g., Verma et al., Alzheimer's Dement. 12:e12030 (2020); which is incorporated by reference herein in its entirety). As such, certain aspects of the present disclosure are directed to methods of administering an EV to a subject in need thereof, comprising administering the EV to the subject by intrathecal injection, followed by applying a mechanical convective force to the torso of the subject. In some aspects, the mechanical convective force is achieved using a high frequency chest wall or lumbothoracic oscillating respiratory clearance device (e.g., a Smart Vest or Smart Wrap, ELECTROMED INC, New Prague, Minn., USA). In some aspects, the mechanical convective force, e.g., the oscillating vest, facilitates spread of the intrathecally dosed EVs further down the nerve thus allowing for better EV delivery to nerves.

In some aspects, the intra- and trans-compartmental biodistribution of exosomes can be manipulated by exogenous extracorporeal forces acting upon a subject after compartmental delivery of exosomes. This includes the application of mechanical convection, for example by way of applying percussion, vibration, shaking, or massaging of a body compartment or the entire body. Following intrathecal dosing for example, the application of chest wall vibrations by several means including an oscillating mechanical jacket can spread the biodistribution of exosomes along the neuraxis or along cranial and spinal nerves, which can be helpful in the treatment of nerve disorders by drug carrying exosomes.

In some aspects, the application of external mechanical convective forces via an oscillating jacket or other similar means can be used to remove exosomes and other material from the cerebrospinal fluid of the intrathecal space and out to the peripheral circulation. This aspect can help remove endogenous toxic exosomes and other deleterious macromolecules such as beta-amyloid, tau, alpha-synuclein, TDP43, neurofilament and excessive cerebrospinal fluid from the intrathecal space to the periphery for elimination.

In some aspects, exosomes delivered via the intracebroventricular route can be made to translocate throughout the neuraxis by simultaneously incorporating a lumbar puncture and allowing for ventriculo-lumbar perfusion wherein additional fluid is infused into the ventricles after exosome dosing, while allowing the existing neuraxial column of CSF to exit is the lumbar puncture. Ventriculo-lumbar perfusion can allow ICV dosed exosome to spread along the entire neuraxis and completely cover the subarachnoid space in order to treat leptomeningeal cancer and other diseases.

In some aspects, the application of external extracorporeal focused ultrasound, thermal energy (heat) or cold may be used to manipulate the compartmental pharmacokinetics and drug release properties of exosomes engineered to be sensitive to these phenomena.

In some aspects, the intracompartmental behavior and biodistribution of exosomes engineered to contain paramagnetic material can be manipulated by the external application of magnets or a magnetic field. In some aspects, the EVs are delivered directly to a tumor. In some aspects, the administration is intratumoral. In some aspects, the composition is administered to the periphery of the tumor.

### V. Producer Cells for Production of Engineered Exosomes

EVs of the present disclosure can be produced from a cell grown in vitro or a body fluid of a subject. When exosomes are produced from in vitro cell culture, various producer cells, e.g., HEK293 cells, CHO cells, and MSCs, can be used. In certain embodiments, a producer cell is not a dendritic cell, macrophage, B cell, mast cell, neutrophil, Kupffer-Browicz cell, cell derived from any of these cells, or any combination thereof

The producer cell can be genetically modified to comprise one or more exogenous sequences (e.g., encoding a vNAR, a scaffold protein, or a therapeutic protein) to produce exosomes described herein. The genetically-modified producer cell can contain the exogenous sequence by transient or stable transformation. The exogenous sequence can be transformed as a plasmid. The exogenous sequences can be stably integrated into a genomic sequence of the producer cell, at a targeted site or in a random site. In some aspects, a stable cell line is generated for production of lumen-engineered exosomes.

The exogenous sequences can be inserted into a genomic sequence of the producer cell, located within, upstream (5′-end) or downstream (3′-end) of an endogenous sequence encoding an exosome protein. Various methods known in the art can be used for the introduction of the exogenous sequences into the producer cell. For example, cells modified using various gene editing methods (e.g., methods using a homologous recombination, transposon-mediated system, loxP-Cre system, CRISPR/Cas9 or TALEN) are within the scope of the present disclosure.

The exogenous sequences can comprise a sequence encoding a scaffold protein disclosed herein or a fragment or variant thereof. An extra copy of the sequence encoding a scaffold protein can be introduced to produce an exosome described herein (e.g., having a higher density of a scaffold protein on the surface, e.g., on the luminal and/or external surface of the EV). An exogenous sequence encoding a modification or a fragment of a scaffold protein can be introduced to produce a lumen-engineered and/or surface-engineered exosome containing the modification or the fragment of the scaffold protein.

In some aspects, a producer cell can be modified, e.g., transfected, with one or more vectors encoding a scaffold protein linked to a vNAR and/or a biologically active moiety, disclosed herein.

In some aspects, a producer cell disclosed herein is further modified to comprise an additional exogenous sequence. For example, an additional exogenous sequence can be introduced to modulate endogenous gene expression, or produce an exosome including a payload (e.g., a vNAR or biologically active moiety). In some aspects, the producer cell is modified to comprise two exogenous sequences, one encoding a scaffold protein, or a variant or a fragment thereof, and the other encoding a payload (e.g., a vNAR or biologically active moiety). In certain aspects, the producer cell can be further modified to comprise an additional exogenous sequence conferring additional functionalities to exosomes. In some aspects, the producer cell is modified to comprise two exogenous sequences, one encoding a scaffold protein disclosed herein, or a variant or a fragment thereof, and the other encoding a vNAR. In some aspects, the producer cell is further modified to comprise one, two, three, four, five, six, seven, eight, nine, or ten or more additional exogenous sequences.

Any of the scaffold moieties described herein can be expressed from a plasmid, an exogenous sequence inserted into the genome or other exogenous nucleic acid, such as a synthetic messenger RNA (mRNA).

In some aspects, the EV and the vNAR are produced by a single cell or a single population of cell types. In some aspects, the EV is produced by a first cell (or a first population of cells), and the vNAR is produced by a second cell (or a second population of cells). In some aspects, the first cell and the second cell are the same type of cell. In some aspects, the first cell and the second cell are not the same type of cell.

### VI. Kits

Also provided herein are kits comprising one or more EVs described herein. In some aspects, provided herein is a pharmaceutical pack or kit comprising one or more containers filled with one or more of the ingredients of the pharmaceutical compositions described herein, such as one or more exosomes provided herein, optional an instruction for use. In some aspects, the kits contain a pharmaceutical composition described herein and any prophylactic or therapeutic agent, such as those described herein.

### VII. Methods of Producing Exosomes

In some aspects, the present disclosure is also directed to methods of producing exosomes described herein. In some embodiments, the method comprises: obtaining the EV from a producer cell, and optionally isolating the obtained EV. In some embodiments, the method comprises: modifying a producer cell by introducing two or more components of an exosome disclosed herein (e.g., a scaffold protein and a vNAR); obtaining the EV from the modified producer cell; and optionally isolating the obtained EV. In further embodiments, the method comprises: obtaining an exosome from a producer cell; isolating the obtained exosome; and modifying the isolated exosome (e.g., by inserting a vNAR). In certain embodiments, the method further comprises formulating the isolated exosome into a pharmaceutical composition.

VII.A. Methods of Modifying a Producer Cell

As described supra, in some embodiments, a method of producing an exosome comprises modifying a producer cell with one or more moieties (e.g., a scaffold protein and/or a vNAR). In certain embodiments, the one or more moieties comprise a vNAR. In some embodiments, the one or more moieties further comprise a scaffold protein disclosed herein.

In some embodiments, the producer cell can be a mammalian cell line, a plant cell line, an insect cell line, a fungi cell line, or a prokaryotic cell line. In certain embodiments, the producer cell is a mammalian cell line. Non-limiting examples of mammalian cell lines include: a human embryonic kidney (HEK) cell line, a Chinese hamster ovary (CHO) cell line, an HT-1080 cell line, a HeLa cell line, a PERC-6 cell line, a CEVEC cell line, a fibroblast cell line, an amniocyte cell line, an epithelial cell line, a mesenchymal stem cell (MSC) cell line, and combinations thereof. In certain embodiments, the mammalian cell line comprises HEK-293 cells, BJ human foreskin fibroblast cells, fHDF fibroblast cells, AGE.HN® neuronal precursor cells, CAP® amniocyte cells, adipose mesenchymal stem cells, RPTEC/TERT1 cells, or combinations thereof. In some embodiments, the producer cell is a primary cell. In certain embodiments, the primary cell can be a primary mammalian cell, a primary plant cell, a primary insect cell, a primary fungi cell, or a primary prokaryotic cell.

In some embodiments, the producer cell is not an immune cell, such an antigen presenting cell, a T cell, a B cell, a natural killer cell (NK cell), a macrophage, a T helper cell, or a regulatory T cell (Treg cell). In other embodiments, the producer cell is not an antigen presenting cell (e.g., dendritic cells, macrophages, B cells, mast cells, neutrophils, Kupffer-Browicz cell, or a cell derived from any such cells).

In some embodiments, the one or more moieties can be a transgene or mRNA, and introduced into the producer cell by transfection, viral transduction, electroporation, extrusion, sonication, cell fusion, or other methods that are known to the skilled in the art.

In some embodiments, the one or more moieties is introduced to the producer cell by transfection. In some embodiments, the one or more moieties can be introduced into suitable producer cells using synthetic macromolecules, such as cationic lipids and polymers (Papapetrou et al., Gene Therapy 12: S118-5130 (2005)). In some embodiments, the cationic lipids form complexes with the one or more moieties through charge interactions. In some of these embodiments, the positively charged complexes bind to the negatively charged cell surface and are taken up by the cell by endocytosis. In some other embodiments, a cationic polymer can be used to transfect producer cells. In some of these embodiments, the cationic polymer is polyethylenimine (PEI). In certain embodiments, chemicals such as calcium phosphate, cyclodextrin, or polybrene, can be used to introduce the one or more moieties to the producer cells. The one or more moieties can also be introduced into a producer cell using a physical method such as particle-mediated transfection, “gene gun”, biolistics, or particle bombardment technology (Papapetrou et al., Gene Therapy 12: S118-S130 (2005)). A reporter gene such as, for example, beta-galactosidase, chloramphenicol acetyltransferase, luciferase, or green fluorescent protein can be used to assess the transfection efficiency of the producer cell.

In certain embodiments, the one or more moieties are introduced to the producer cell by viral transduction. A number of viruses can be used as gene transfer vehicles, including moloney murine leukemia virus (MMLV), adenovirus, adeno-associated virus (AAV), herpes simplex virus (HSV), lentiviruses, and spumaviruses. The viral mediated gene transfer vehicles comprise vectors based on DNA viruses, such as adenovirus, adeno-associated virus and herpes virus, as well as retroviral based vectors.

In certain embodiments, the one or more moieties are introduced to the producer cell by electroporation. Electroporation creates transient pores in the cell membrane, allowing for the introduction of various molecules into the cell. In some embodiments, DNA and RNA as well as polypeptides and non-polypeptide therapeutic agents can be introduced into the producer cell by electroporation.

In certain embodiments, the one or more moieties introduced to the producer cell by microinjection. In some embodiments, a glass micropipette can be used to inject the one or more moieties into the producer cell at the microscopic level.

In certain embodiments, the one or more moieties are introduced to the producer cell by extrusion.

In certain embodiments, the one or more moieties are introduced to the producer cell by sonication. In some embodiments, the producer cell is exposed to high intensity sound waves, causing transient disruption of the cell membrane allowing loading of the one or more moieties.

In certain embodiments, the one or more moieties are introduced to the producer cell by cell fusion. In some embodiments, the one or more moieties are introduced by electrical cell fusion. In other embodiments, polyethylene glycol (PEG) is used to fuse the producer cells. In further embodiments, sendai virus is used to fuse the producer cells.

In some embodiments, the one or more moieties are introduced to the producer cell by hypotonic lysis. In such embodiments, the producer cell can be exposed to low ionic strength buffer causing them to burst allowing loading of the one or more moieties. In other embodiments, controlled dialysis against a hypotonic solution can be used to swell the producer cell and to create pores in the producer cell membrane. The producer cell is subsequently exposed to conditions that allow resealing of the membrane.

In some embodiments, the one or more moieties are introduced to the producer cell by detergent treatment. In certain embodiments, producer cell is treated with a mild detergent which transiently compromises the producer cell membrane by creating pores allowing loading of the one or more moieties. After producer cells are loaded, the detergent is washed away thereby resealing the membrane.

In some embodiments, the one or more moieties introduced to the producer cell by receptor mediated endocytosis. In certain embodiments, producer cells have a surface receptor which upon binding of the one or more moieties induces internalization of the receptor and the associated moieties.

In some embodiments, the one or more moieties are introduced to the producer cell by filtration. In certain embodiments, the producer cells and the one or more moieties can be forced through a filter of pore size smaller than the producer cell causing transient disruption of the producer cell membrane and allowing the one or more moieties to enter the producer cell.

In some embodiments, the producer cell is subjected to several freeze thaw cycles, resulting in cell membrane disruption allowing loading of the one or more moieties.

VII.B. Methods of Modifying an Exosome

In some embodiments, a method of producing an exosome comprises modifying the isolated exosome by directly introducing one or more moieties into the EVs. In certain embodiments, the one or more moieties comprise a vNAR. In some embodiments, the one or more moieties comprise a scaffold protein disclosed herein.

In certain embodiments, the one or more moieties are introduced to the exosome by transfection. In some embodiments, the one or more moieties can be introduced into the EV using synthetic macromolecules such as cationic lipids and polymers (Papapetrou et al., Gene Therapy 12: S118-S130 (2005)). In certain embodiments, chemicals such as calcium phosphate, cyclodextrin, or polybrene, can be used to introduce the one or more moieties to the EV.

In certain embodiments, the one or more moieties are introduced to the EV by electroporation. In some embodiments, exosomes are exposed to an electrical field, which causes transient holes in the EV membrane, allowing loading of the one or more moieties.

In certain embodiments, the one or more moieties are introduced to the EV by microinjection. In some embodiments, a glass micropipette can be used to inject the one or more moieties directly into the EV at the microscopic level.

In certain embodiments, the one or more moieties are introduced to the EV by extrusion.

In certain embodiments, the one or more moieties are introduced to the EV by sonication. In some embodiments, EVs are exposed to high intensity sound waves, causing transient disruption of the EV membrane allowing loading of the one or more moieties.

In some embodiments, one or more moieties can be conjugated to the surface of the EV. Conjugation can be achieved chemically or enzymatically, by methods known in the art.

In some embodiments, the EV comprises one or more moieties that are chemically conjugated. Chemical conjugation can be accomplished by covalent bonding of the one or more moieties to another molecule, with or without use of a linker. The formation of such conjugates is within the skill of artisans and various techniques are known for accomplishing the conjugation, with the choice of the particular technique being guided by the materials to be conjugated. In certain embodiments, polypeptides are conjugated to the EV. In some embodiments, non-polypeptides, such as lipids, carbohydrates, nucleic acids, and small molecules, are conjugated to the EV.

In some embodiments, the one or more moieties are introduced to the EV by hypotonic lysis. In such embodiments, the EVs can be exposed to low ionic strength buffer causing them to burst allowing loading of the one or more moieties. In other embodiments, controlled dialysis against a hypotonic solution can be used to swell the EV and to create pores in the EV membrane. The EV is subsequently exposed to conditions that allow resealing of the membrane.

In some embodiments, the one or more moieties are introduced to the EV by detergent treatment. In certain embodiments, extracellular vesicles are treated with a mild detergent, which transiently compromises the EV membrane by creating pores allowing loading of the one or more moieties. After EVs are loaded, the detergent is washed away thereby resealing the membrane.

In some embodiments, the one or more moieties are introduced to the EV by receptor mediated endocytosis. In certain embodiments, EVs have a surface receptor, which upon binding of the one or more moieties induces internalization of the receptor and the associated moieties.

In some embodiments, the one or more moieties are introduced to the EV by mechanical firing. In certain embodiments, extracellular vesicles can be bombarded with one or more moieties attached to a heavy or charged particle such as gold microcarriers. In some of these embodiments, the particle can be mechanically or electrically accelerated such that it traverses the EV membrane.

In some embodiments, extracellular vesicles are subjected to several freeze thaw cycles, resulting in EV membrane disruption allowing loading of the one or more moieties.

VII.C. Methods of Isolating an EV

In some embodiments, methods of producing EVs disclosed herein comprises isolating the EV from the producer cells. In certain embodiments, the EVs released by the producer cell into the cell culture medium. It is contemplated that all known manners of isolation of EVs are deemed suitable for use herein. For example, physical properties of EVs can be employed to separate them from a medium or other source material, including separation on the basis of electrical charge (e.g., electrophoretic separation), size (e.g., filtration, molecular sieving, etc.), density (e.g., regular or gradient centrifugation), Svedberg constant (e.g., sedimentation with or without external force, etc.). Alternatively, or additionally, isolation can be based on one or more biological properties, and include methods that can employ surface markers (e.g., for precipitation, reversible binding to solid phase, FACS separation, specific ligand binding, non-specific ligand binding, affinity purification etc.).

Isolation and enrichment can be done in a general and non-selective manner, typically including serial centrifugation. Alternatively, isolation and enrichment can be done in a more specific and selective manner, such as using EV or producer cell-specific surface markers. For example, specific surface markers can be used in immunoprecipitation, FACS sorting, affinity purification, and magnetic separation with bead-bound ligands.

In some embodiments, size exclusion chromatography can be utilized to isolate the EVs. Size exclusion chromatography techniques are known in the art. Exemplary, non-limiting techniques are provided herein. In some embodiments, a void volume fraction is isolated and comprises the EVs of interest. Further, in some embodiments, the EVs can be further isolated after chromatographic separation by centrifugation techniques (of one or more chromatography fractions), as is generally known in the art. In some embodiments, for example, density gradient centrifugation can be utilized to further isolate the extracellular vesicles. In certain embodiments, it can be desirable to further separate the producer cell-derived EVs from EVs of other origin. For example, the producer cell-derived EVs can be separated from non-producer cell-derived EVs by immunosorbent capture using an antigen antibody specific for the producer cell.

In some embodiments, the isolation of EVs can involve combinations of methods that include, but are not limited to, differential centrifugation, size-based membrane filtration, immunoprecipitation, FACS sorting, and magnetic separation.

The practice of the present disclosure will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Sambrook et al., ed. (1989) Molecular Cloning A Laboratory Manual (2nd ed.; Cold Spring Harbor Laboratory Press); Sambrook et al., ed. (1992) Molecular Cloning: A Laboratory Manual, (Cold Springs Harbor Laboratory, NY); D. N. Glover ed., (1985) DNA Cloning, Volumes I and II; Gait, ed. (1984) Oligonucleotide Synthesis; Mullis et al. U.S. Pat. No. 4,683,195; Hames and Higgins, eds. (1984) Nucleic Acid Hybridization; Hames and Higgins, eds. (1984) Transcription And Translation; Freshney (1987) Culture Of Animal Cells (Alan R. Liss, Inc.); Immobilized Cells And Enzymes (IRL Press) (1986); Perbal (1984) A Practical Guide To Molecular Cloning; the treatise, Methods In Enzymology (Academic Press, Inc., N.Y.); Miller and Calos eds. (1987) Gene Transfer Vectors For Mammalian Cells, (Cold Spring Harbor Laboratory); Wu et al., eds., Methods In Enzymology, Vols. 154 and 155; Mayer and Walker, eds. (1987) Immunochemical Methods In Cell And Molecular Biology (Academic Press, London); Weir and Blackwell, eds., (1986) Handbook Of Experimental Immunology, Volumes I-IV; Manipulating the Mouse Embryo, Cold Spring Harbor Laboratory Press, Cold Spring Harbor, N.Y., (1986)); Crooke, Antisense drug Technology: Principles, Strategies and Applications, 2^{nd} Ed. CRC Press (2007) and in Ausubel et al. (1989) Current Protocols in Molecular Biology (John Wiley and Sons, Baltimore, Md.).

The following examples are offered by way of illustration and not by way of limitation.

### EXAMPLES

### Example 1—Construction and Characterization of Exosomes Surface-Loaded with CD47

Several constructs were created expressing an anti-TfR vNAR moeity fused to PTGFRn, a scaffold X protein. In each construct, the vNAR sequence (vNAR-1, vNAR-2, or vNAR-3; Table 4) was fused to the N-terminus of PTGFRN. Additionally, mEGFP, FLAG, and HiBiT were fused to the C-terminus of the PTGFRN to allow for validation, biodistribution, and activity analysis. Ultimately, three constructs were created: pUC57-AAVS1-vNAR-anti-TfnR PTGFRN-mEGP-FLAG-HiBiT; pUC57-AAVS1-vNAR-anti-TfnR-2-PTGFRN-mEGP-FLAG-HiBiT; and pUC57-AAVS1-vNAR-anti-TfnR-3-PTGFRN-mEGP-FLAG-HiBiT. Two control constructs were similarly created that comprise a vNAR that does not specifically bind TfR.

TABLE 4
Anti-TfRvNAR Sequences

| | |
|---|---|
| vNAR- | ARVDQTPQTITKETGESLTINCVLRDNDCTLSSTHWYRKKSG |
| anti- | STNEERISKGGRYVETVNSGSKSFSLRINDLTVEDSGTYRCN |
| TfnR-1 | VYAMTRNWWCDVYGGGTVVTVNAASGA (SEQ ID NO: |
| | 193) |
| vNAR- | ARVDQTPQTITKETGESLTINCVLRDSNCALSSTYWYRKKSG |
| anti- | STKEESISKGGRYVETVNSGSKSFSLRINDLTVEDSGTYRCN |
| TfnR-2 | VWHDLVWSVCTTDVYGGGTVVTVNAASGA (SEQ ID NO: |
| | 194) |
| vNAR- | ARVDQTPQTITKEEGESLTINCVLRDSSSALASTSWYRKKSG |
| anti- | STREETISKGGRYVETVNSGSKSFSLRINDLTVEDSGTYRCN |
| TfnR-3 | VYELVEDTSAYEIGVDVYGDGTAVTVNAASGA (SEQ ID |
| | NO: 195) |

### Example 2—Construction and Characterization of Exosomes Surface-Loaded with CD47

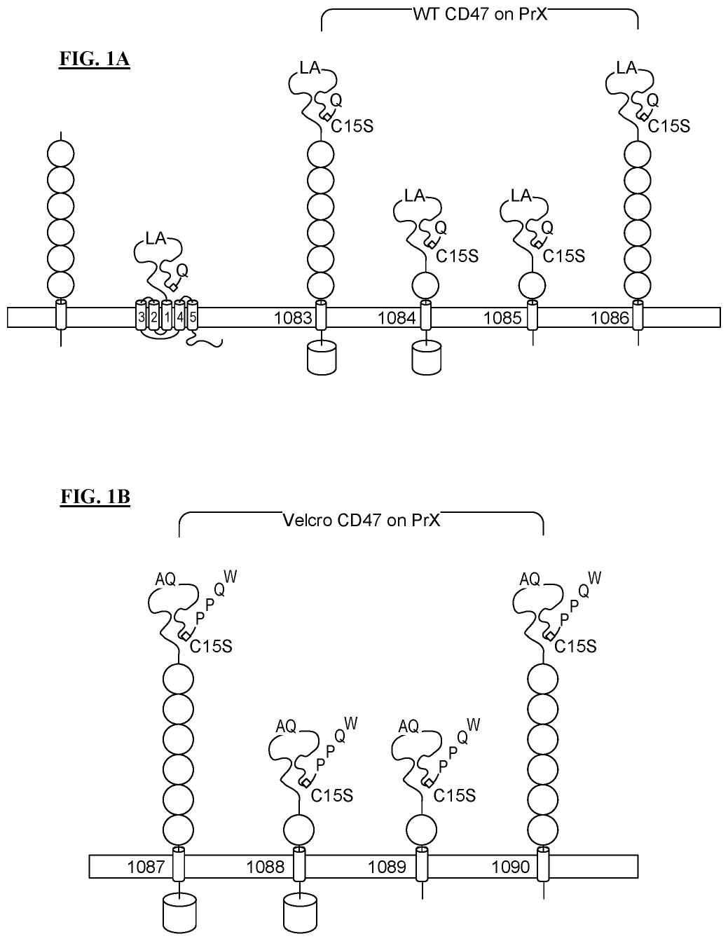

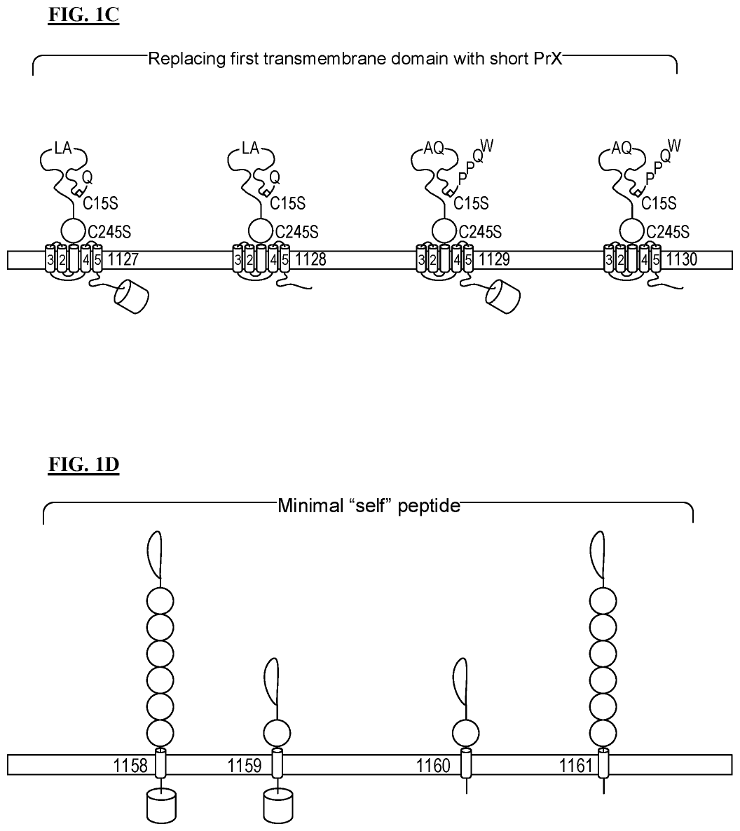

In order to minimize the uptake of administered exosomes by native myeloid cells, various constructs were created to load human CD47 or a fragment thereof on the surface of exosomes. The extracellular domain of human wild type CD47, having a C15S substitution, or Velcro-CD47 was fused to Scaffold X or a fragment thereof and expressed in exosome-producing cells (FIGS. 1A-1B). In addition, exosomes were produced expressing a modified CD47 having a truncated Scaffold X protein inserted in the first domain of of human wild type CD47, having a C15S substitution, or Velcro-CD47 (FIG. 1C). Further exosomes were generated expressing a minimal “self” peptide (GNYTCEVTELTREGETIIELK; SEQ ID NO: 160) fused to Scaffold X or a fragment thereof (FIG. 1D; see, e.g., Rodriguez et al., Science 339:971-75 (February 2013)).

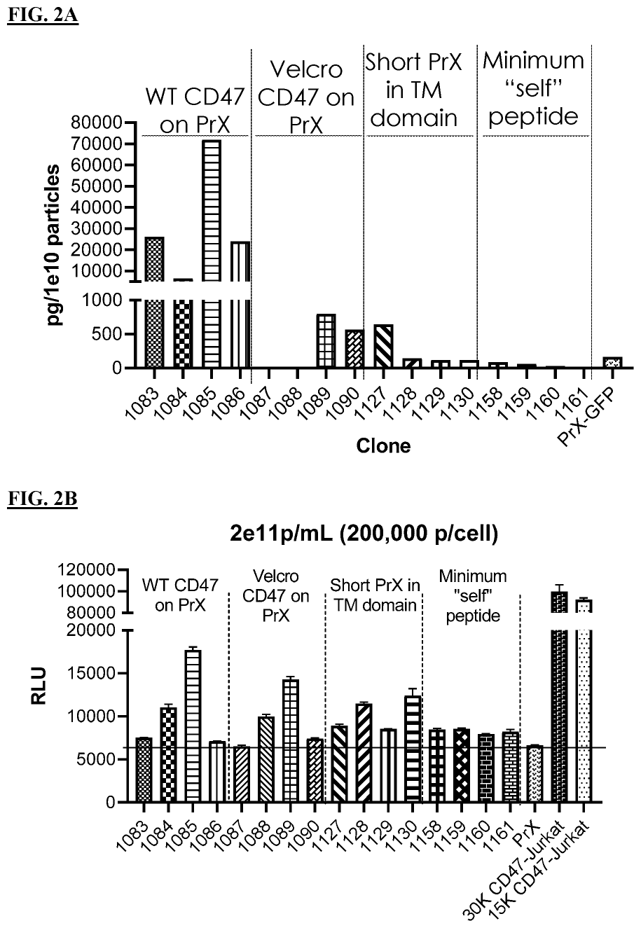

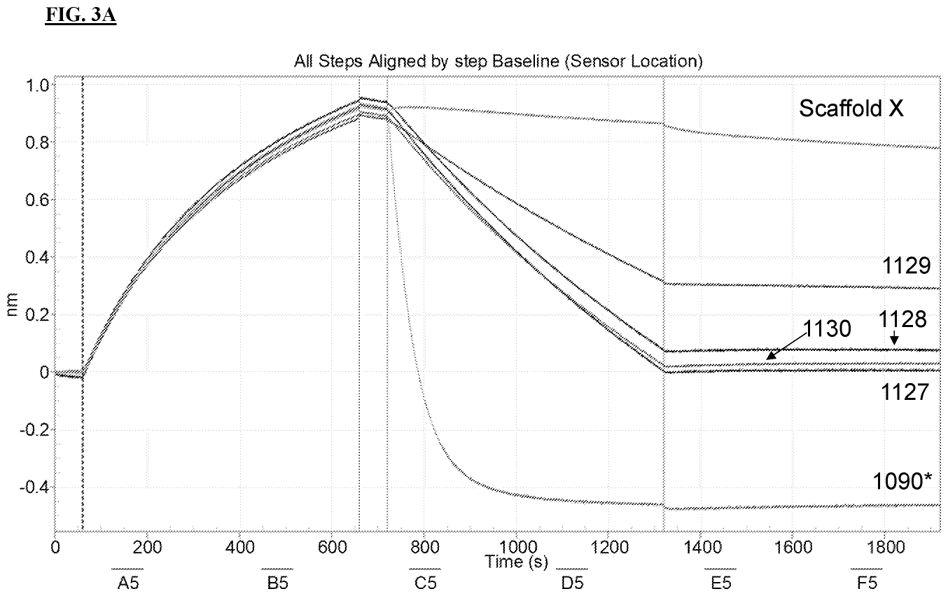

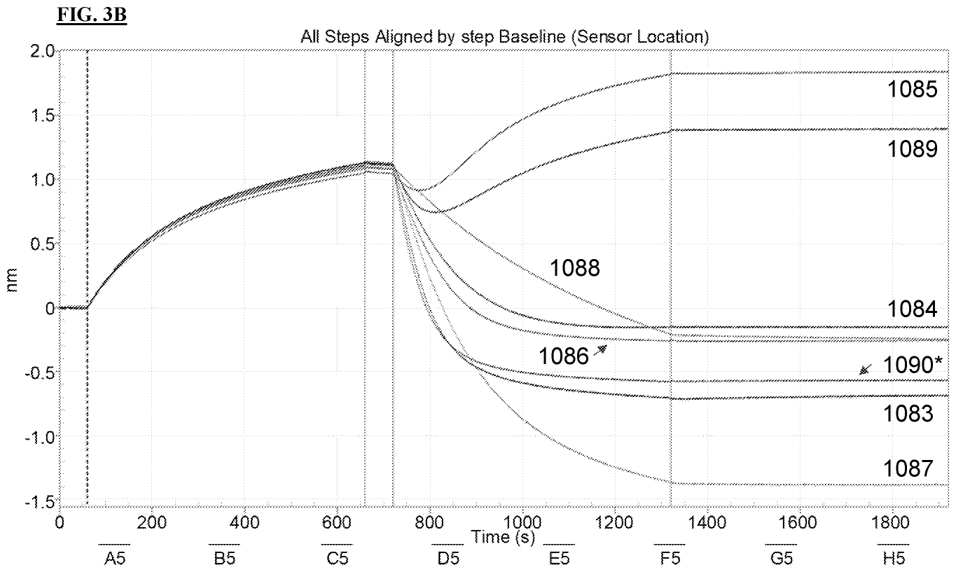

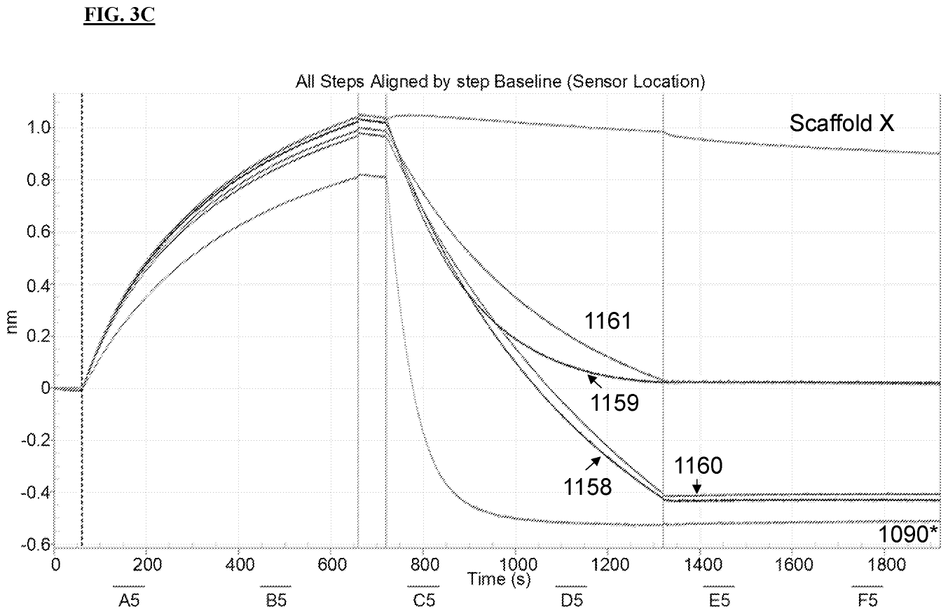

Exosomes loaded with each construct were assayed for CD47 expression by ELISA using an anti-CD47 antibody targeted to a specific epitope of CD47 (FIG. 2A) or by binding to SIRPα using a SIRPα (human) signaling reporter cell bioassay (DiscoverX) (FIG. 2B) or using Octet analysis (FIGS. 3A-3C). Because the ELISA antibody recognized a specific epitope of CD47, some constructs were not recognized in the ELISA experiments. The results of each method of assaying CD47 expression are summarized in Table 5.

TABLE 5
Summary of CD47 Exosome Expression Assays

| Construct | ELISA | Bioassay | Octet |
|---|---|---|---|
| 1083 | Y | | Y |
| 1084 | Y | Y | Y |
| 1085 | Y | Y | Y |
| 1086 | Y | | Y |
| 1087 | | | Y |
| 1088 | | Y | Y |
| 1089 | Y | Y | Y |
| 1090 | Y | | Y |
| 1127 | Y | Low | Y |
| 1128 | | Y | Y |
| 1129 | | Low | Y |
| 1130 | | Y | Y |
| 1158 | | Low | Y |
| 1159 | | Low | Y |
| 1160 | | Low | Y |
| 1161 | | Low | Y |
| PrX | | | |

### Example 3—In Vitro Analysis of Uptake of Exosomes Surface Loaded with CD47

Monocytes were isolated from blood and differentiated to macrophages by culturing for 7-8 days in M-CSF. For each CD47 construct, exosomes were labeled with pHrodo-Red with NHS chemistry (3 rounds of 30 minutes). Exosomes were passed through a 70 μm qEV column and subjected through ultracentrifucation to further clean and concentrate the exosomes. Particle concentration was determined by NTA. Macrophages were re-plated, and exosomes were added to the macrophage culture at varying concentrations. Cells were imaged using IncuCyte, with 3 fields of view per well.

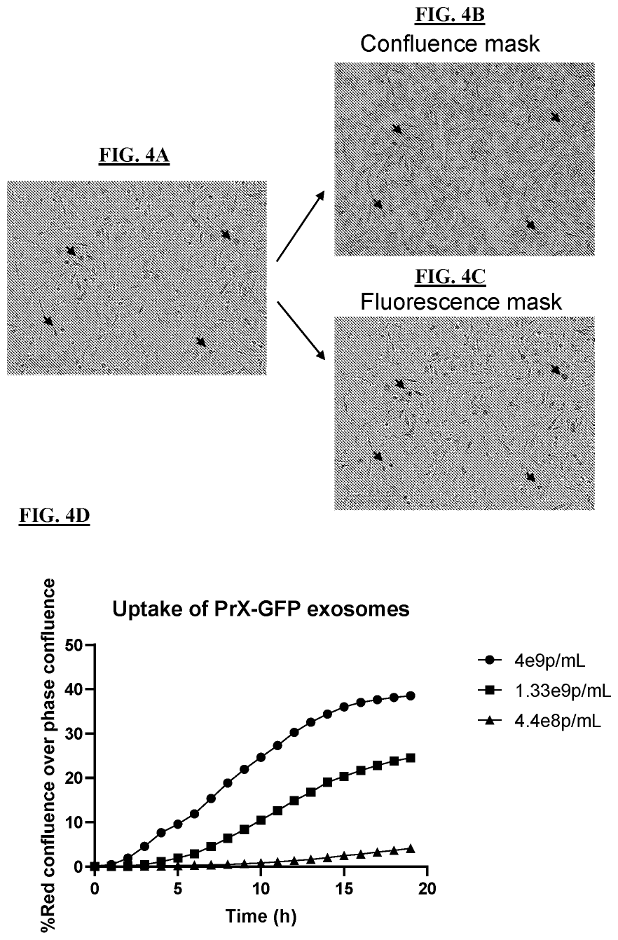

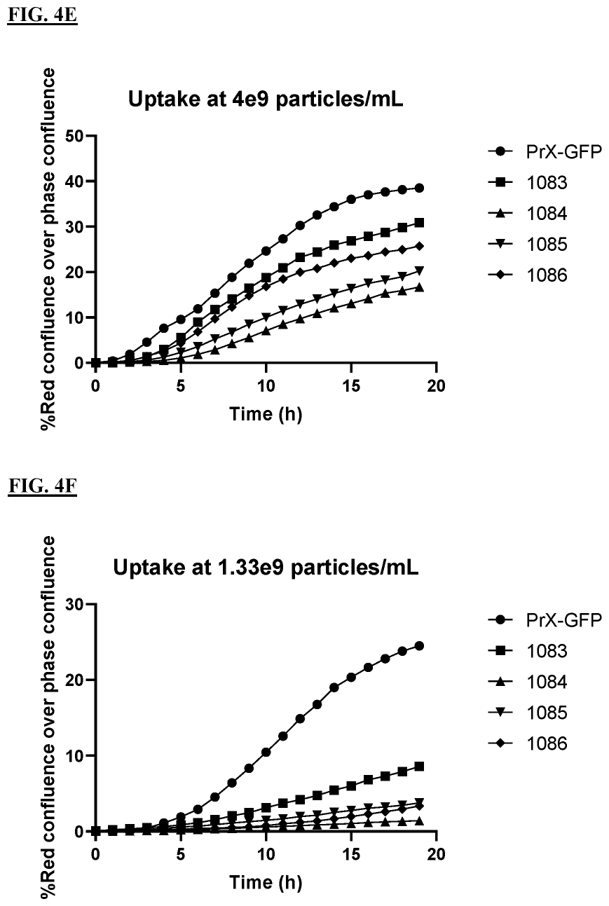

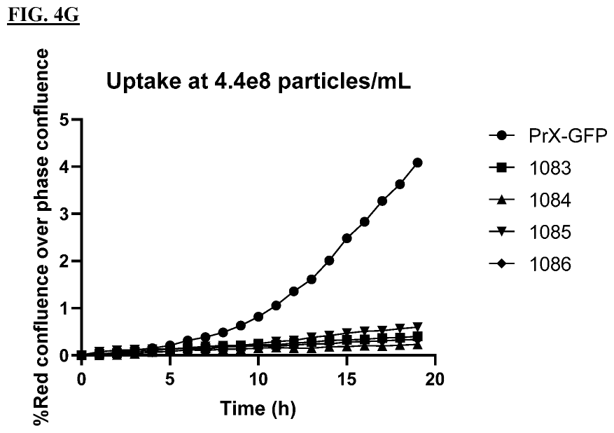

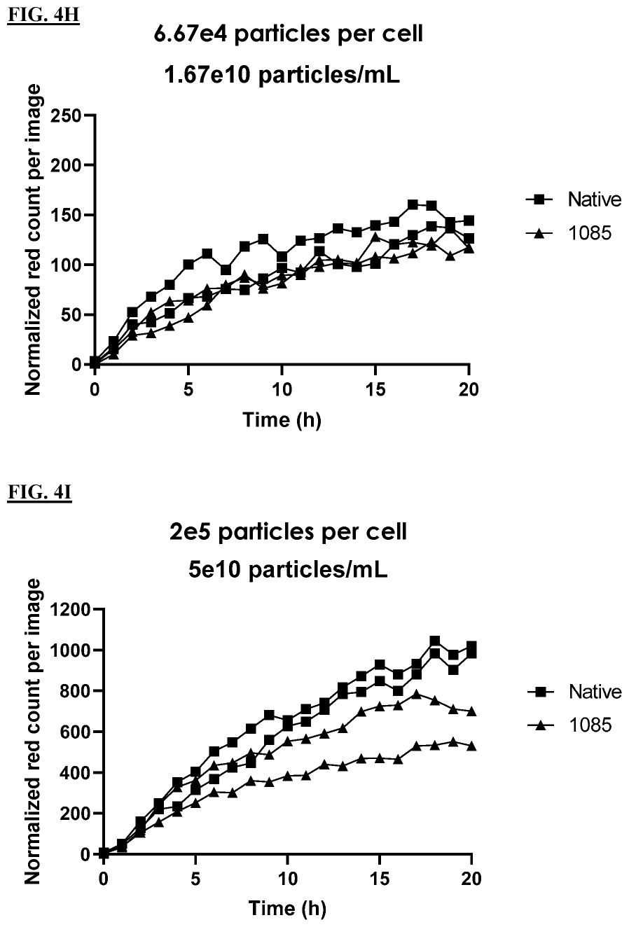

Dose-dependent uptake of control exosomes expressing GFP fused to Scaffold X by primary human monocyte-derived M0 macrophages was observed (FIGS. 4A-4D). However, expression of CD47 on the exosome surface resulted in decreased uptake of the exosomes by primary human monocyte-derived M0 macrophages, relative to the Scaffold X-GFP controls (FIGS. 4E-4H), with little to no effect on the uptake of the exosomes by adherent HEK cells, which do not express SIRPα (FIGS. 4I-4J).

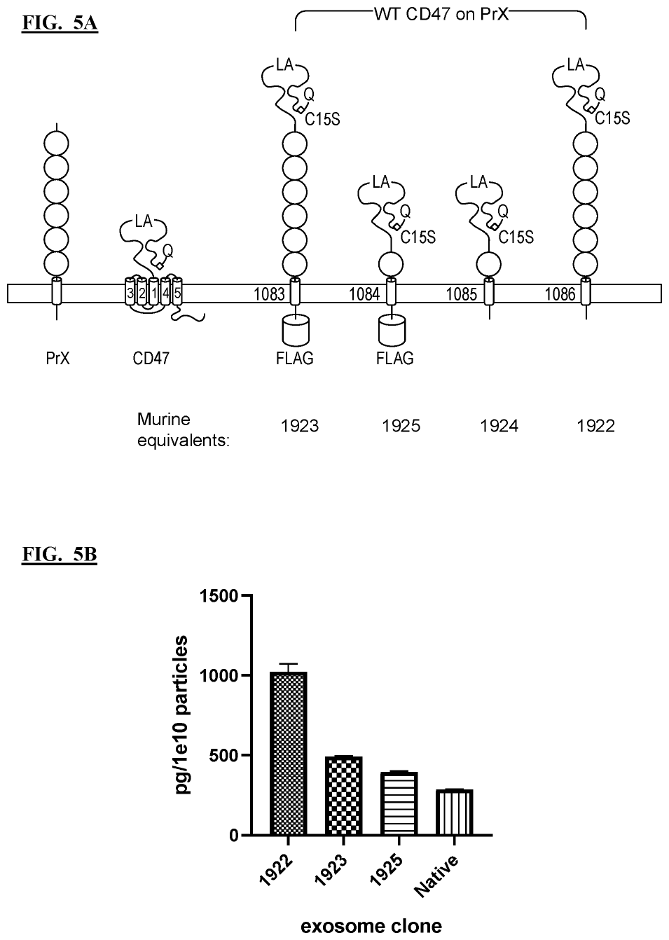

To test the effect of surface-expressed CD47 in mouse models, flag-tagged (1923 and 1925) and non-flag-tagged (1922 and 1924) constructs were created by fusing the extracellular domain of murine CD47^{C15S} to a full length scaffold X protein (1922 and 1923) or a truncated scaffold X protein (1924 and 1925) (FIG. 5A). The constructs were expressed in exosome producing cells, and exosomes were isolated and screened for CD47 protein expression and activity. Surface-expression of CD47 was observed for each of the constructs (FIGS. 5B-5C). Octet assay showed binding of both human and mouse CD47 exosomes to mouse SIRPα (FIG. 6). With the exception of hCD47 construct pCB-1085, the mouse and other human CD47 exosomes showed binding to mouse SIRPα.

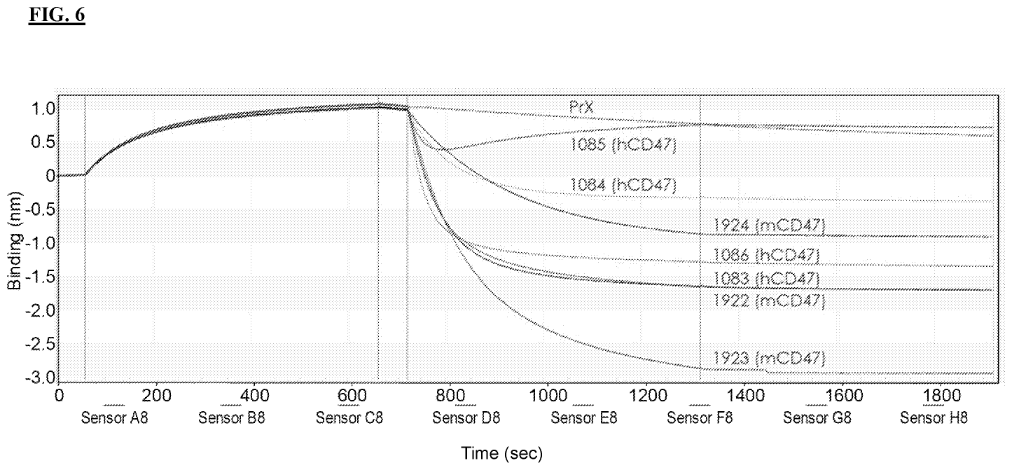

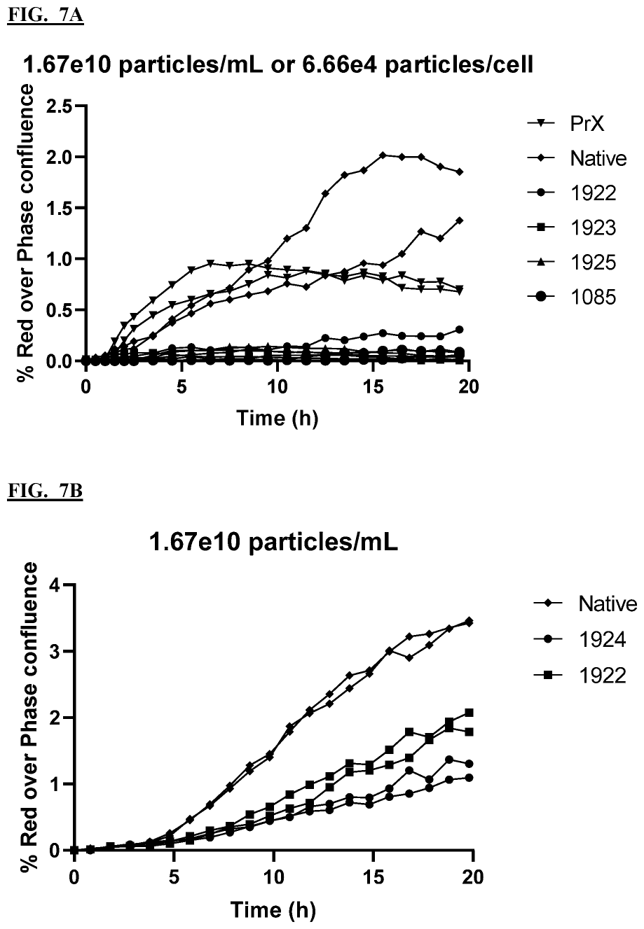

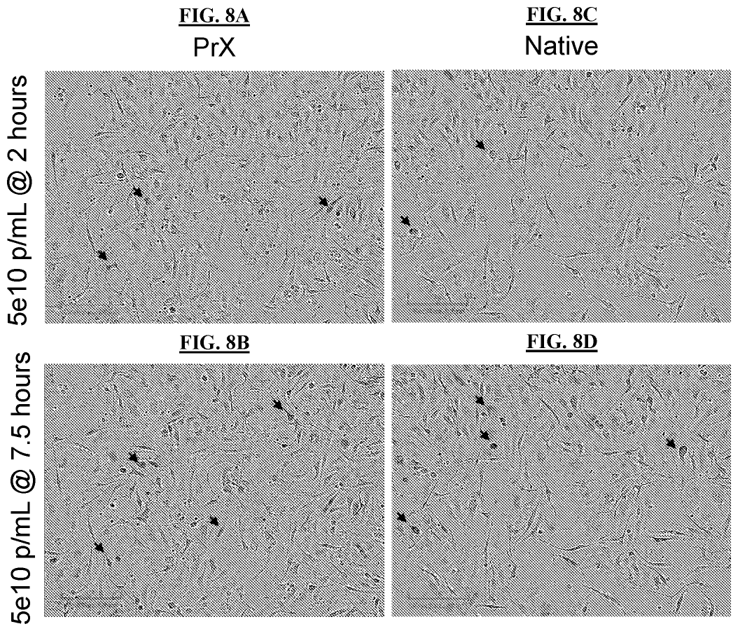

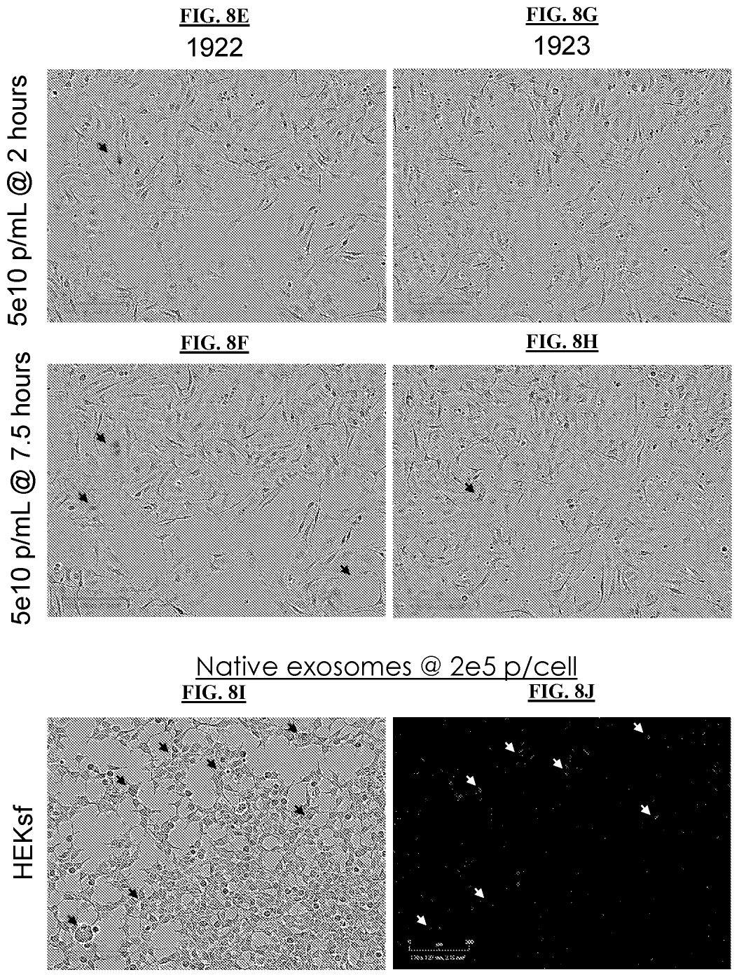

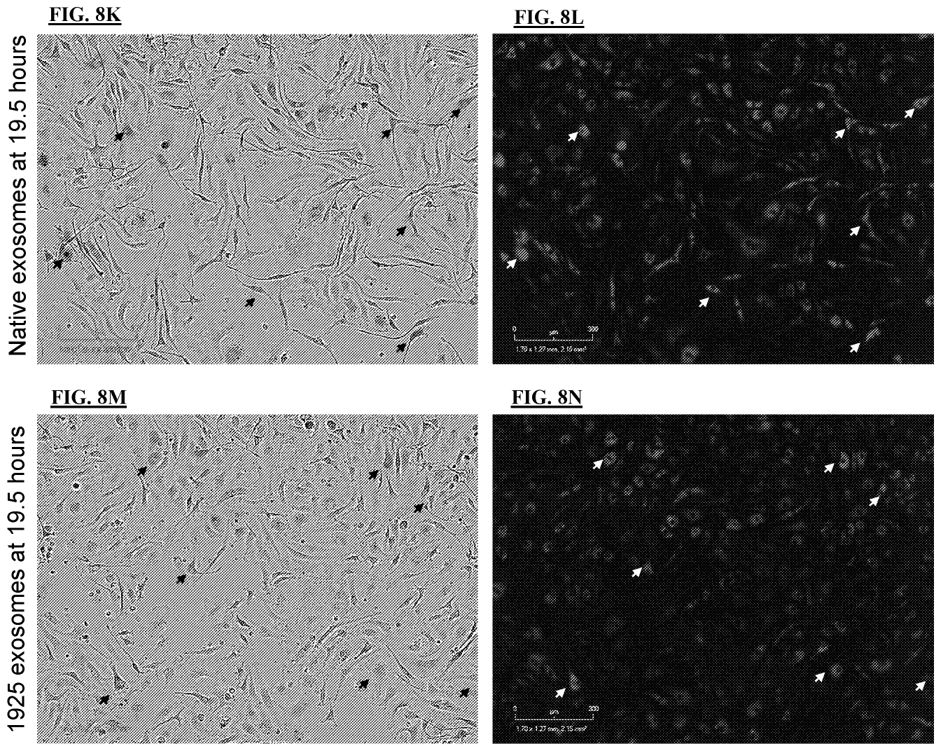

In vitro, mCD47-expressing exosomes displayed decreased uptake by SIRPα⁺ mouse bone marrow-derived macrophages (BMDM; FIGS. 7A-7B; 8A-8N). In addition, the human construct 1085 also reduce uptake of exosomes in the mouse bone marrow-derived macrophages.

### Example 4—Construction and Characterization of Exosomes with Tropism Moieties

In order to direct EVs to specific cellular types, various constructs were created to express different tropism moieties. To determine whether the RVG peptide could be used to direct EVs to neurons, several constructs were tested. The constructs tested were: RVG-PrX-mCherry-FLAG-HiBiT (construct 2021), linker-PrX-mCherry-FLAG-HiBiT (construct 2022), RVG-LAMP2B-mCherry-FLAG-HiBiT (construct 2023), and linker-LAMP2B-mCherry-FLAG-HiBiT (construct 2024).

“RVG” refers to a tropism moiety of having the amino acid sequence YTIWMPENPRPGTPCDIFTNSRGKRASNG (SEQ ID NO: 165). “Linker” refers to a linker having the amino acid sequence GGSSGSGSGSGGGGSGGGGTGTSSSGTGT (SEQ ID NO: 192). “FLAG” refers to a FLAG® epitope tag. “HiBiT” refers to a nano luciferase peptide. “mCherry” is a red fluorescent protein. “LAMP2B” and “PrX” are protein scaffolds, e.g., as described above. “ExoRVG” EVs are exosomes comprising an RVG tropism moiety.

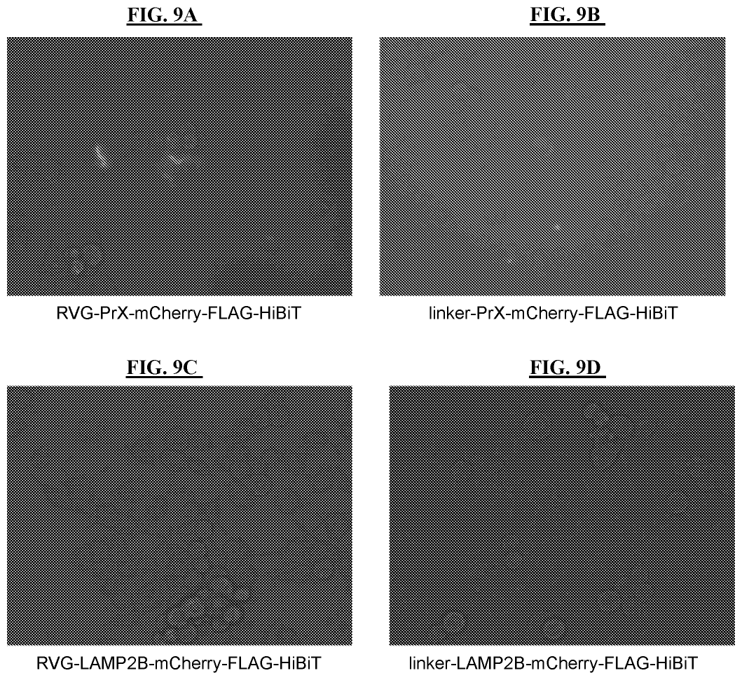

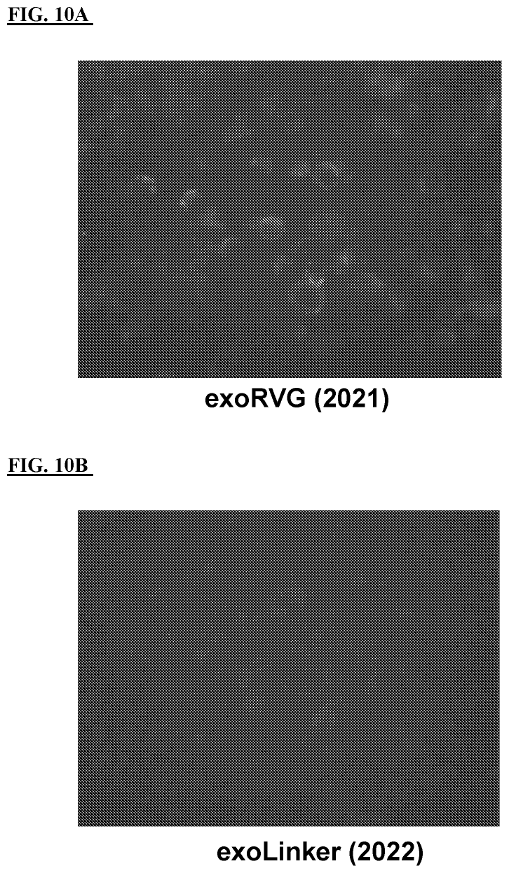

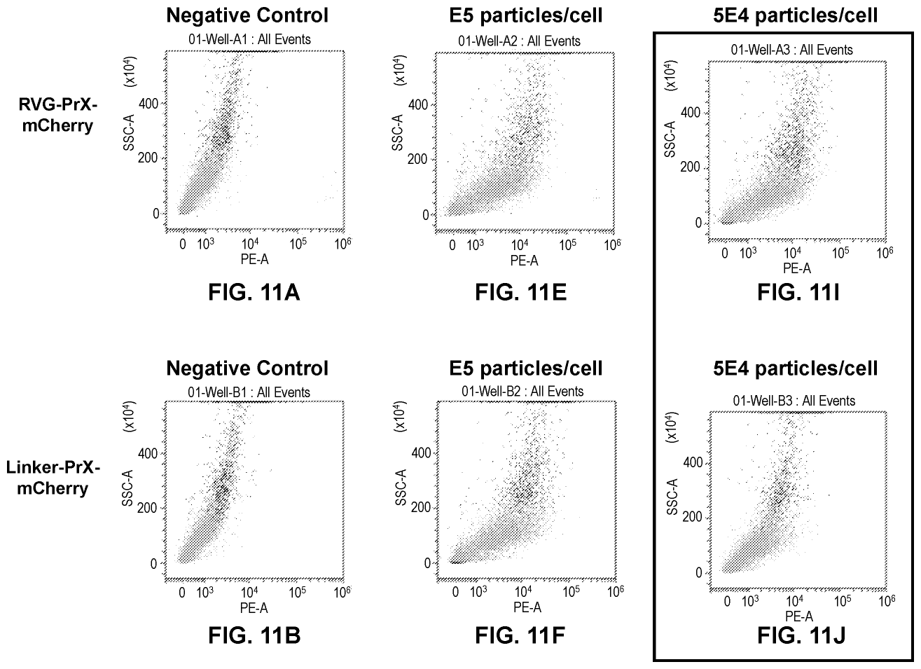

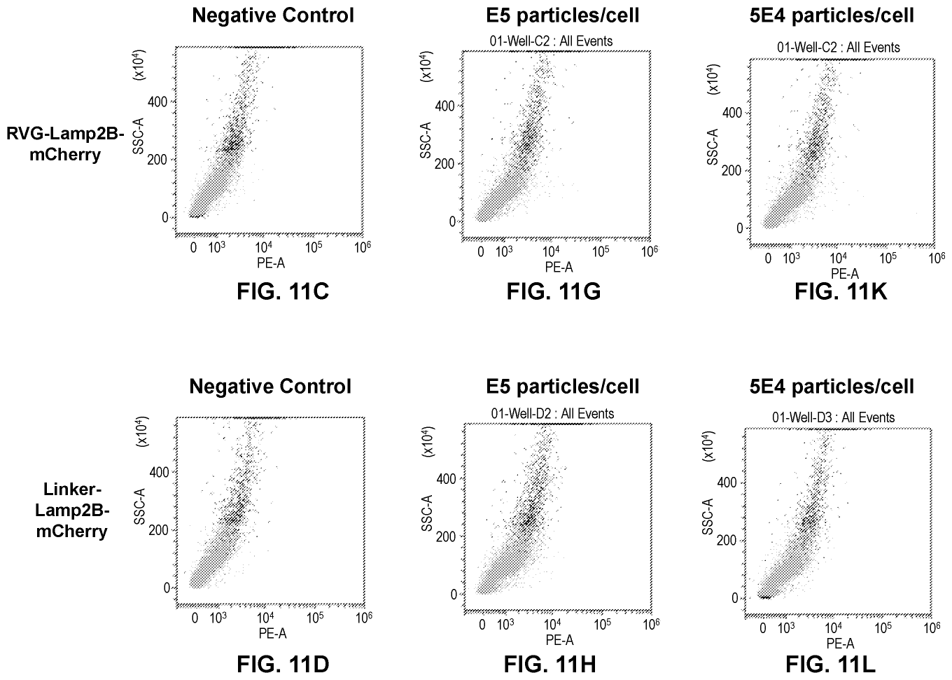

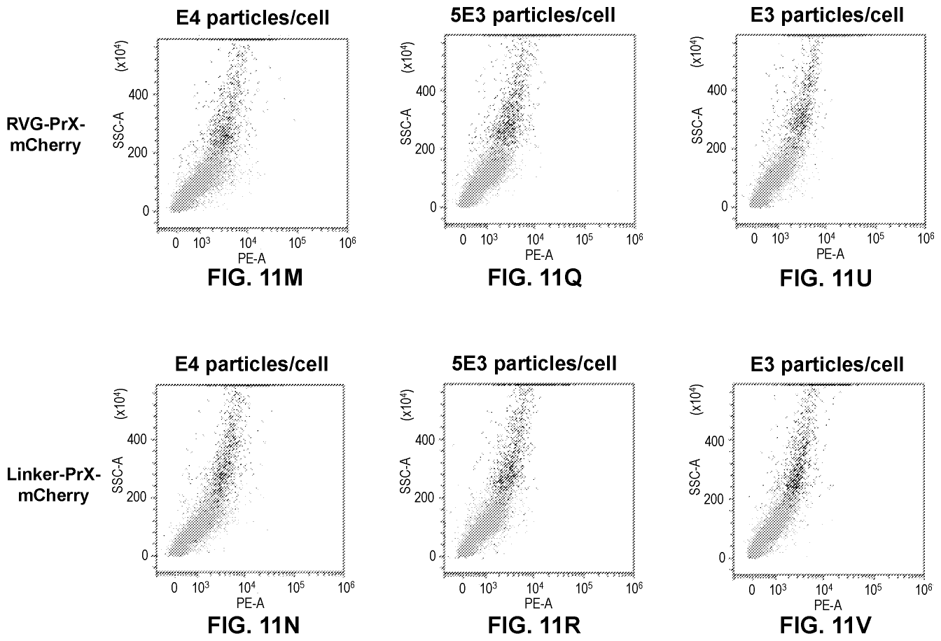

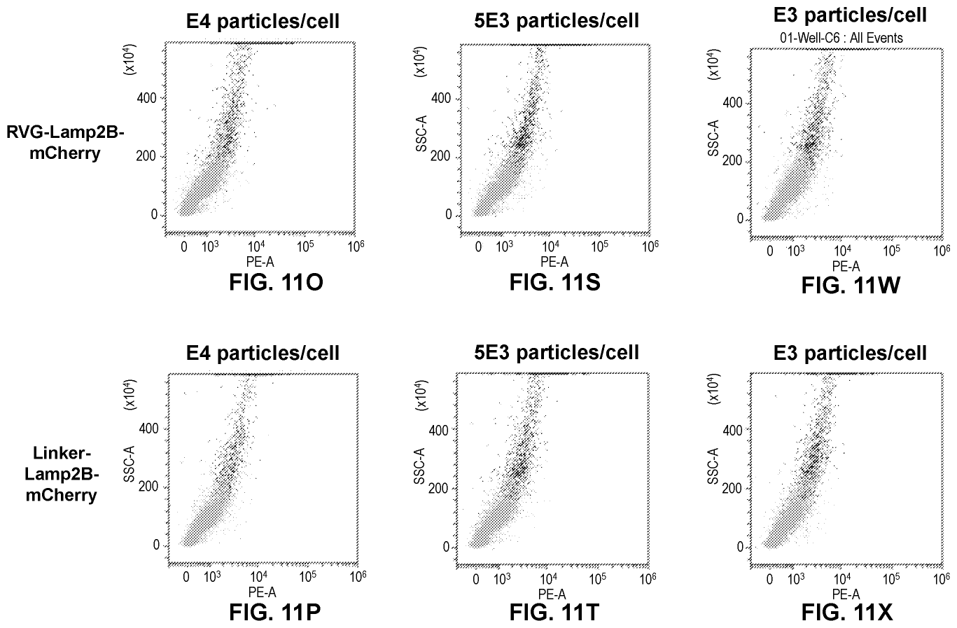

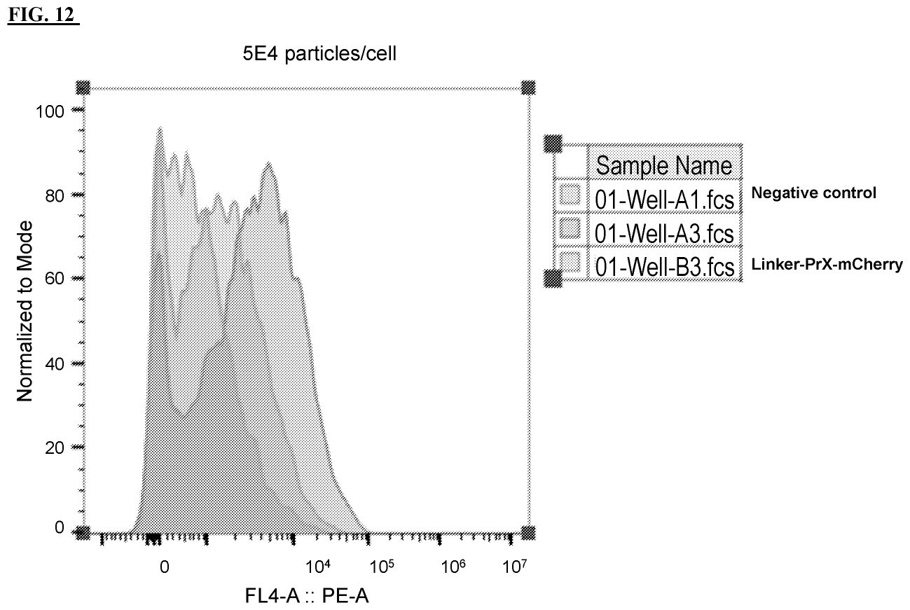

Neuro2A cells were incubated with 10⁵, 5×10⁴, 10⁴, 5×10³, or 10³ EV particles comprising the constructs disclosed above per neuron2A cell, and mCherry fluorescence was measured using microscopy. No obvious signal was observed at 1 hour or 2 hours after adding the EVs. However, EV uptake was observed at 5 hours with 10⁵ EV particles/neuro2A cell (FIGS. 9A-9D). Only the constructs comprising RVG showed uptake by the neuro2A cells. Increased uptake was observed after 18 hours (FIGS. 10A-10B). Flow cytometry showed significant uptake of UVs comprising RVG after 24 hours, both at 10⁵ EV particles/neuro2A cell and at 5×10⁴ EV particles/neuro2A cell (FIGS. 11A-11X and 12). These results indicated that attaching an RVG peptide to the external surface of an EV, e.g., an exosome, can target the EVs to neurons.

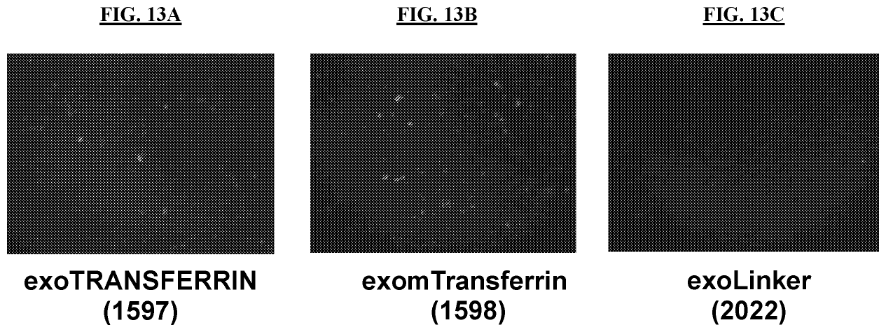

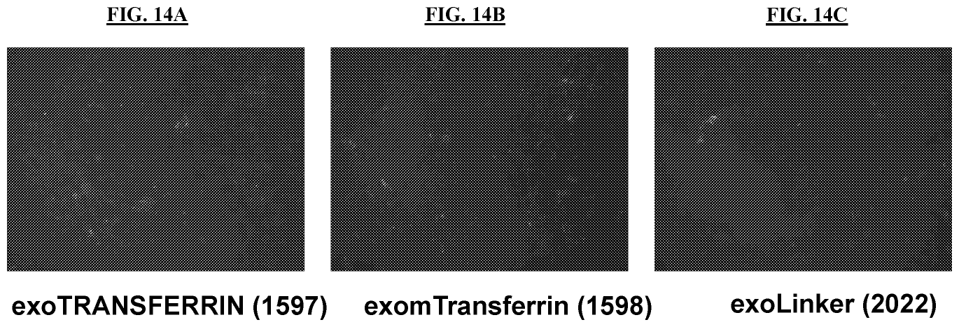

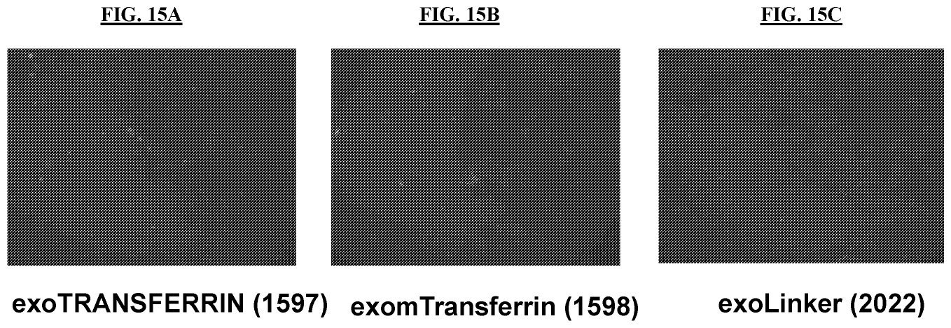

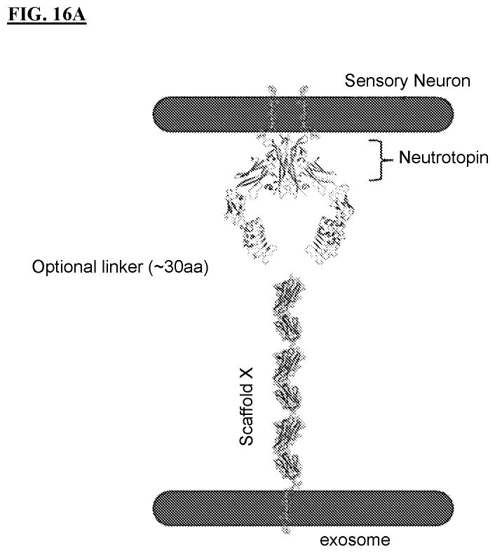

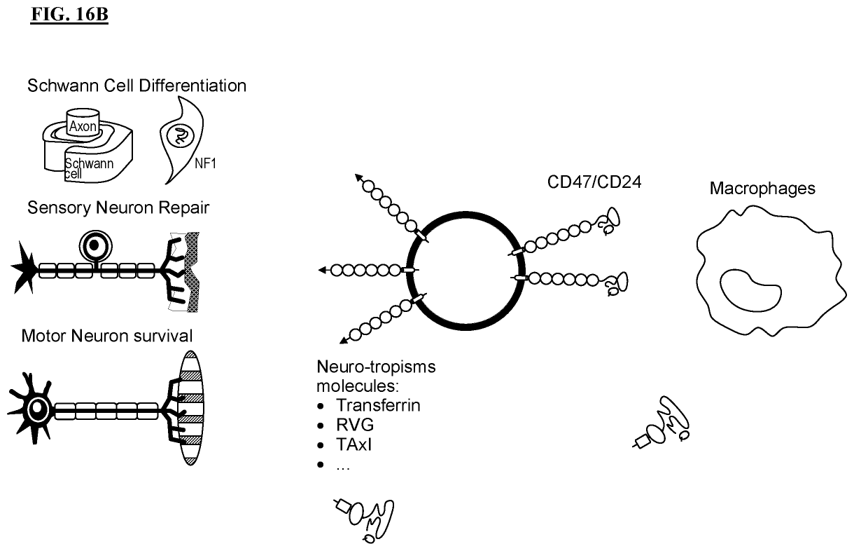

A second tropism moiety, transferrin, was also evaluated. Several constructs were tested: Transferrin-PrX-mCherry-FLAG (comprising human transferrin) (construct 1597), mTransferrin-PrX-mCherry-FLAG (comprising mouse transferrin) (construct 1598); and linker-PrX-mCherry-FLAG-HiBiT (construct 2022). 5×10⁵ EV particles per cell were used. Uptake was measured 3 hours after EV particle incubation started. Uptake was measured using microscopy. EV uptake by HeLa cells (FIGS. 13A-13C), Hep3B cells (FIG. 14A-14C) and Hep3G2 cells (FIGS. 15A-15C) was observed for both human and mouse transferrin-containing EVs, indicating that transferrin can be use to target EVs to these three cell types.

### Example 6—Engineered Exosomes Targeting Neuronal Cells

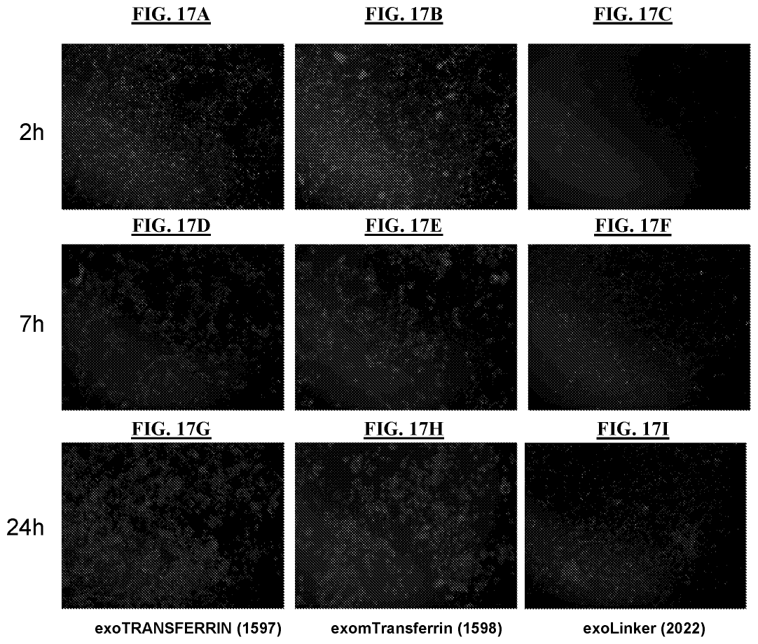

Neuro2A cells were seeded at E5 cells/well in 24-well plate and incubated with EVs expressing exoTransferrin at 5×105 EV particles/cell. EVs were engineered with mCherry tag, and mCherry fluorescence was examined by fluorescent microscopy (FIGS. 17A-171). Exo-mTransferrin and exoTransferrin showed faster (starting after 2h, increased at 7h and even high at 24h)) and greater uptake by the neuro2A cells than the exoLinker negative control. These results indicated that attaching Transferrin to the external surface of an EV, can enhance the EVs uptake in neuronal cells.

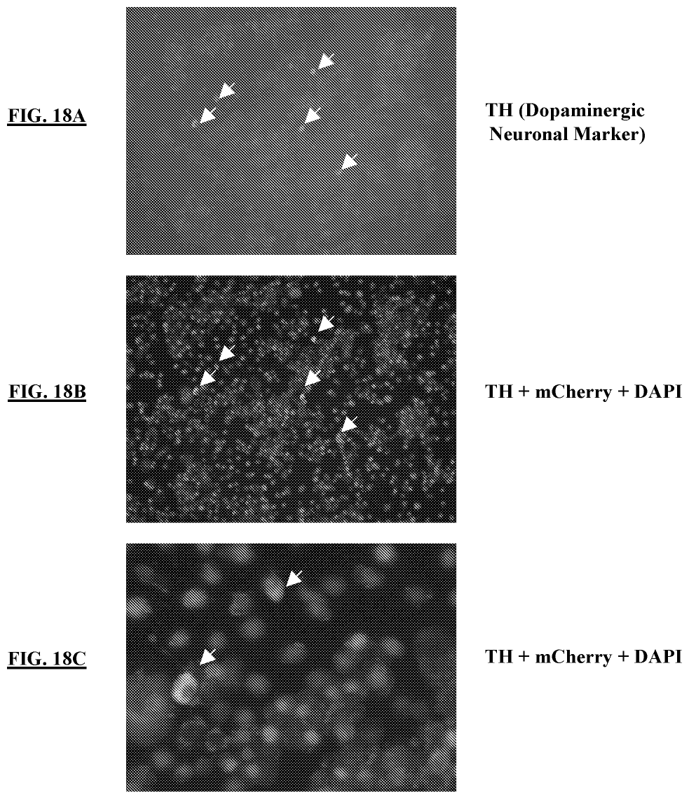

Further, exo-Transferrin showed uptake by the differentiated neuro2A cells overnight (FIGS. 18A-18C). These results indicated that attaching transferrin to the external surface of an EV, can enhance the EVs uptake in neuronal cells.

Human neuroblastoma cells, SH-SY-5Y, were seeded at E5 cells per well in 24-well plate and incubated with EV samples for 24h. Exo-mTransferrin showed greater uptake by the SH-SY-5Y cells than the exoLinker negative control (FIGS. 19A-19B). These results indicate that attaching Transferrin to the external surface of an EV, can enhance the EVs uptake in human neuronal cells.

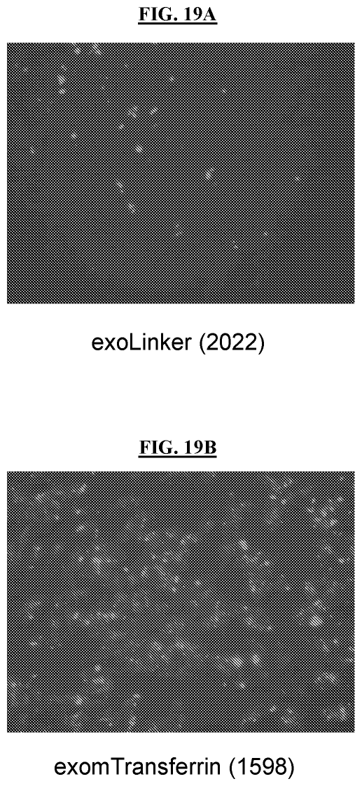

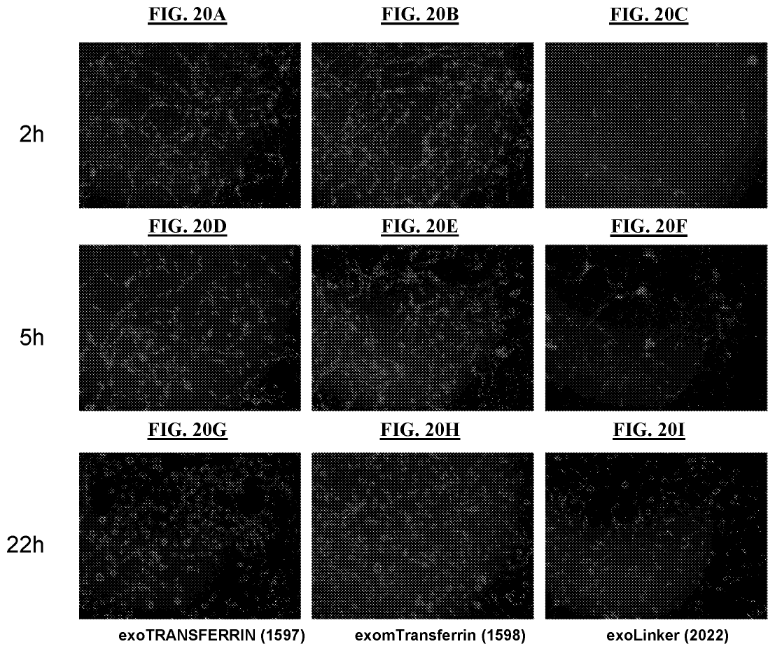

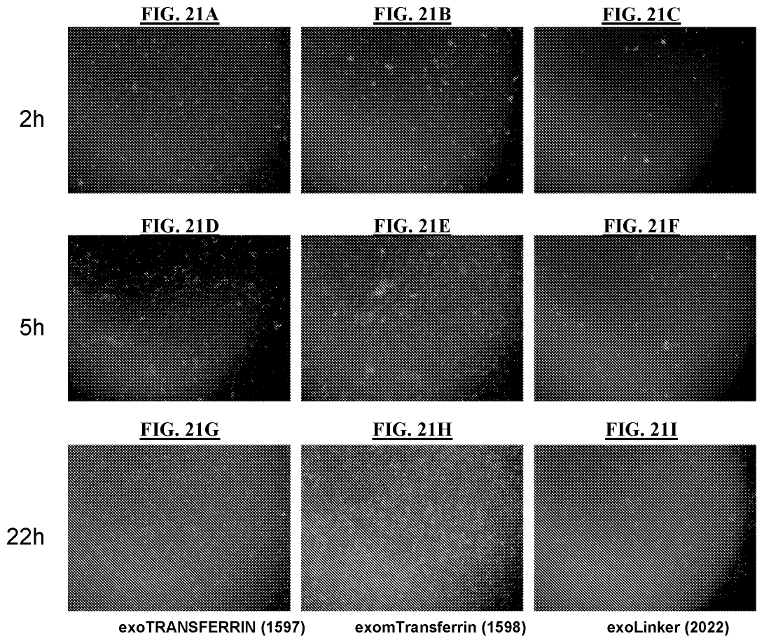

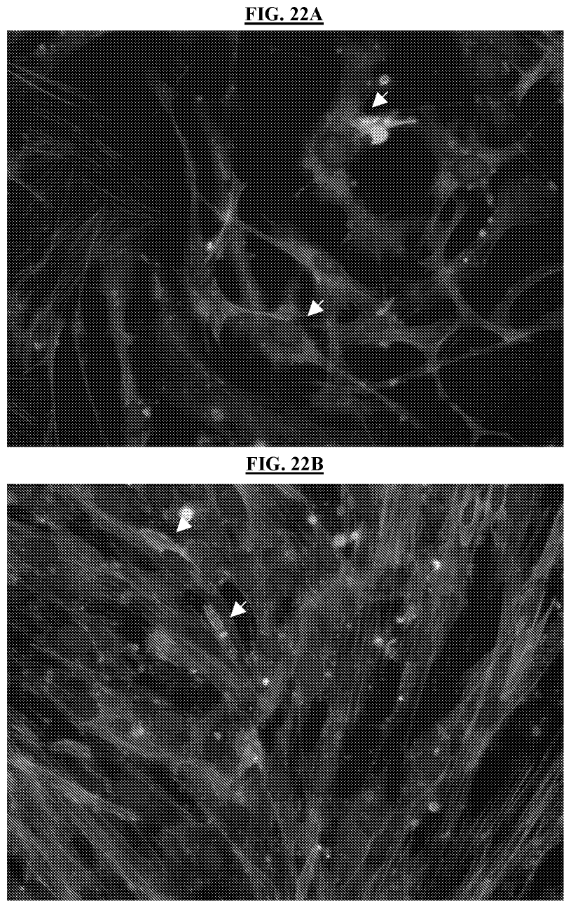

Primary mouse Schwann cells (ScienCell) were seeded at E5 cells/well into 24 well plate, incubated with 5×10⁵ EV particles/cell. EVs were engineered with mCherry tag and mCherry fluorescence were examined by fluorescent microscopy. Exo-mTransferrin and exoTransferrin showed faster (starting after 2h, increased at 7h and 22h)) and greater uptake by the mouse Schwann cells than the exoLinker negative control (FIGS. 20A-20I). These results indicated that attaching Transferrin to the external surface of an EV, can enhance the EVs uptake in Schwann cells. Primary human Schwann cells (ScienCell) were then seeded at E5 cells/well into 24-well plate, incubated with 5×10⁵ EV particles/cell. Evs were engineered with mCherry tag and mCherry fluorescence was examined by fluorescent microscopy. Exo-mTransferrin and exoTransferrin showed greater uptake by the human Schwann cells compared to the exoLinker negative control at 5h and 22h time points (FIGS. 21A-21I). These results indicated that attaching Transferrin to the external surface of an EV, can enhance the Evs uptake in human Schwann cells. ExoTransferrin showed uptake in both mouse and human Schwann cells. Fixed cells were stained with anti-cytoskeleton-marker antibody and DAPI, and imaging reveals that the exoTransferrin EVs were taken up more by mouse Schwann cells (FIG. 22A) than human Schwann cells (FIG. 22B).

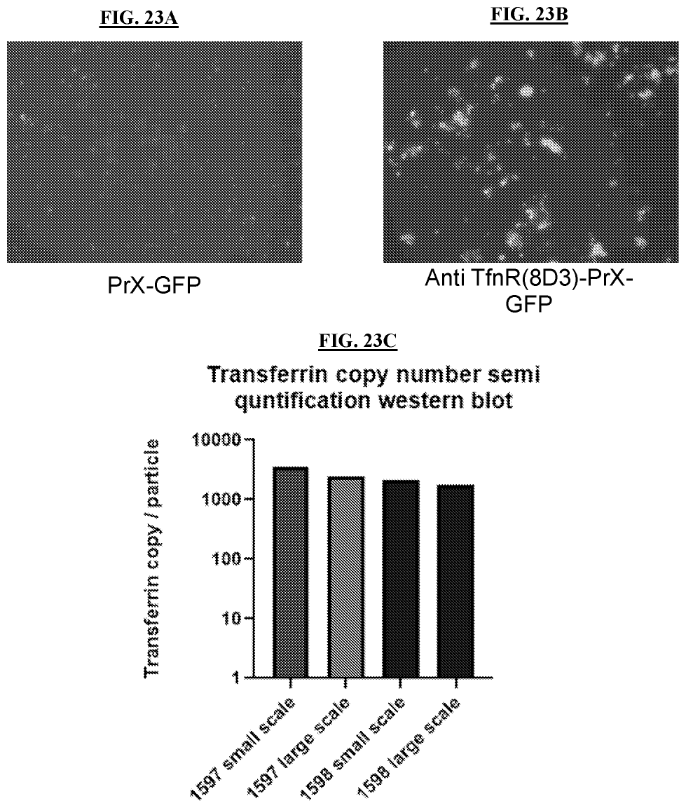

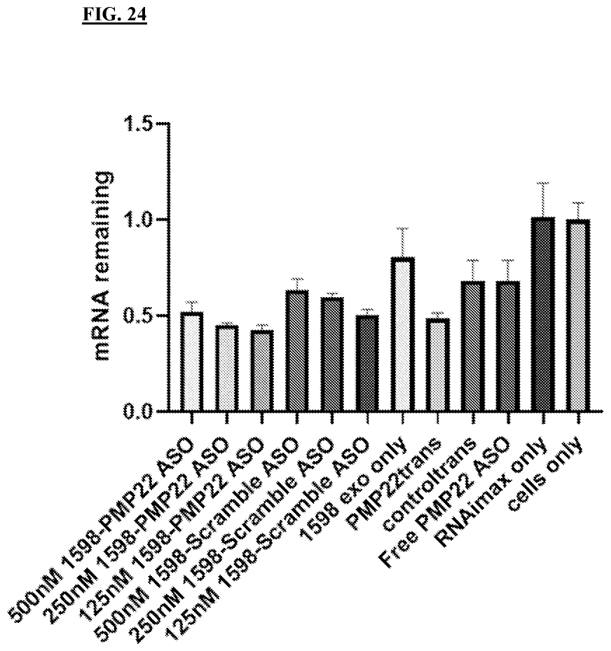

The anti-Transferrin receptor antibody (8D3) was tested as a means of targeting EVs to human neuroblasts cells. SH-SY-5Y cells were cultured a E5 cells/well in the presense of EVs expressing PrX-GFP (negative control) or anti-TfnR(8D3)-PrX-GFP (FIG. 23C) overnight. anti-TfnR(8D3)-PrX-GFP EVs were readily taken up by the neuroblast cells (FIG. 23B).

### Example 7—PMP22 Knockdown—ASO Exosome Delivery

X61832, i.e., a PMP22 ASO of sequence CTCATTCGCGTTTCCGC (SEQ ID NO: 146) was delivered to mouse Schwann cells using exosomes expressing mouse transferrin on their surfaces (exo-mTransferrin exosomes). FIG. 28 shows that delivery of the PMP22 ASO using exo-mTransferrin exosomes resulted in a significantly higher knockdown that the knockdown observed when the ASO was administers without exosomes, or when the ASO was administering using exosomes that did not express transferrin.

### Example 8—Engineered Exosomes Targeting Muscle Cells

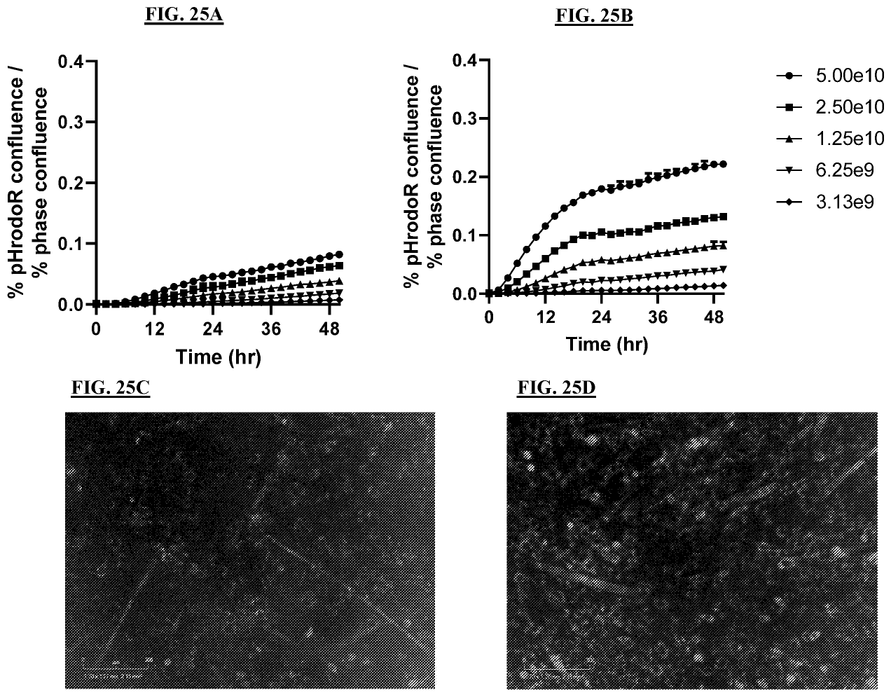

To evaluate the uptake of exosomes by muscle cells, differentiated C2C12 myotubes were incubated in vitro with exosomes overexpressing PTGFRN (PrX) or exosomes engineered to display mouse transferrin (mTrf). Exosomes were labelled with pHrodoR dye, which fluoresces upon exposure to acidic pH. The percentage of pHrodoR positive signal is normalized to the cell confluence. Dose titrations of each construct were prepared ranging from 5E10 p/mL to 3.13E9 p/mL (FIGS. 25A-25B). Exosomes engineered to display mTrf exhibited enhanced uptake compared to control PrX exosomes (FIGS. 25A-25D). Representative fluorescence microscopy images 24 hours post-treatment are shown in FIG. 25C (exosomes overexpressing PTGFRN) and FIG. 25D (exosomes engineered to display mouse transferrin.

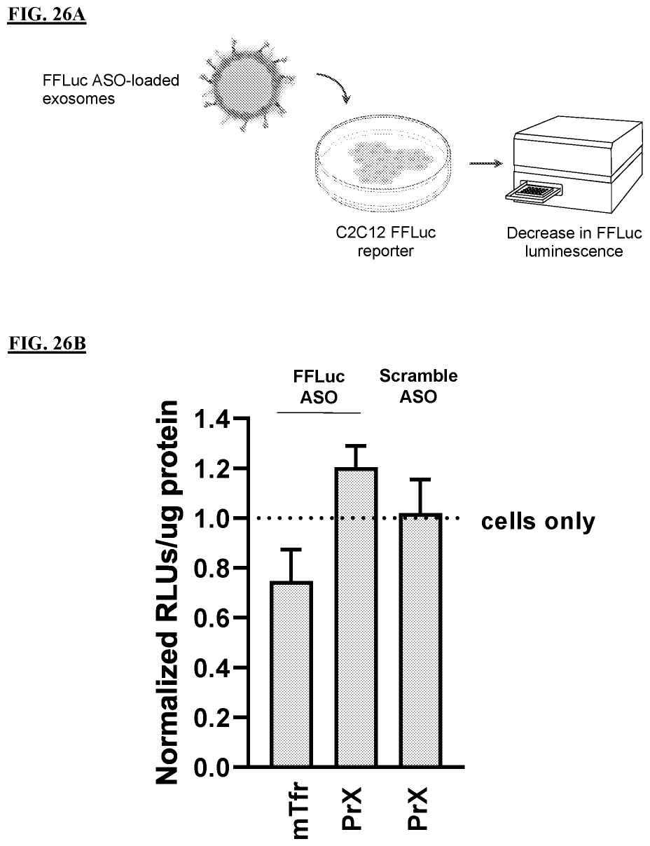

Exosomes engineered to display mouse transferrin were loaded with ASOs targeting firefly luciferase (FFLuc) or a scrambled control sequence (FIG. 26A). C2C12 reporter cells engineered to overexpress FFLuc were treated with the ASO-loaded exosomes. Luminescence activity was measured 72 hours post-treatment and data were normalized to total protein to account for differences in cell growth. Approximately 25% knockdown was achieved with mouse transferrin-displaying exosomes compared to cells only (FIG. 26B).

### INCORPORATION BY REFERENCE

All publications, patents, patent applications and other documents cited in this application are hereby incorporated by reference in their entireties for all purposes to the same extent as if each individual publication, patent, patent application or other document were individually indicated to be incorporated by reference for all purposes.

### EQUIVALENTS

While various specific aspects have been illustrated and described, the above specification is not restrictive. It will be appreciated that various changes can be made without departing from the spirit and scope of the disclosure(s). Many variations will become apparent to those skilled in the art upon review of this specification.

What is claimed is:

1. An extracellular vesicle (EV) comprising an antigen-binding moeity that specifically binds transferrin receptor (TfR), wherein the antigen-binding moeity is loaded on the exterior surface of the EV.

2. The EV of claim 1, wherein the antigen-binding moeity specifically binds human TfR.

3. The EV of claim 1 or 2, wherein the antigen-binding moeity increases the transport of the EV across the blood brain barrier in a human subject.

4. The EV of claim 3, wherein the antigen-binding moeity increases the transport of the EV across the blood brain barrier by at least about 10%, at least about 20%, at least about 30%, at least about 40%, at least about 75%, at least about 100%, at least about 150%, at least about 200%, at least about 250%, at least about 300%, at least about 350%, at least about 400%, at least about 450%, at least about 500%, at least about 600%, at least about 700%, at least about 800%, at least about 900%, or at least about 1000%, as compared to a reference EV not comprising an antigen-binding moeity that specifically binds human TfR.

5. The EV of any one of claims 1 to 4, wherein the antigen-binding moeity an antibody or an antigen-binding portion thereof.

6. The EV of any one of claims 1 to 4, wherein the antigen-binding moeity comprises one or more single-domain antigen-binding moieties.

7. The EV of claim 6, wherein the antigen-binding moeity comprises at least two single-domain antigen-binding moieties.

8. The EV of claim 7, wherein the at least two single-domain antigen-binding moieties are the same.

9. The EV of claim 7, wherein the at least two single-domain antigen-binding moieties are different.

10. The EV of claim 9, wherein the at least two single-domain antigen-binding moieties comprise (i) a first single-domain antigen-binding moiety that binds a first epitope on TfR and (ii) a second single-domain antigen-binding moiety binds a second epitope on TfR.

11. The EV of any one of claims 6 to 10, wherein the antigen-binding moeity comprises at least three, at least four, at least five, or at least 6 single-domain antigen-binding moieties.

12. The EV of any one of claims 6 to 11, wherein each of the one or more single-domain antigen-binding moieties are linked to each other by a linker.

13. The EV of any one of claims 6 to 12, wherein the one or more single-domain antigen-binding moieties are selected from a VHH, a vNAR, an antigen-binding fragment of a VHH, an antigen-binding fragment of a vNAR, and any combination thereof.

14. The EV of claim 7, wherein the vNAR or the fragment thereof is derived from an IgNAR.

15. The EV of claim 7, wherein the vNAR or the fragment thereof is synthetic.

16. The EV of claim 7, wherein the VHH or the fragment thereof is derived from a camelid antibody.

17. The EV of claim 7, wherein the VHH or the fragment thereof is synthetic.

18. The EV of any one of claims 6 to 17, wherein the single-domain antigen-binding moiety increases the permeability of the EV across the blood brain barrier by at least about 10%, at least about 20%, at least about 30%, at least about 40%, at least about 75%, at least about 100%, at least about 150%, at least about 200%, at least about 250%, at least about 300%, at least about 350%, at least about 400%, at least about 450%, at least about 500%, at least about 600%, at least about 700%, at least about 800%, at least about 900%, or at least about 1000%, as compared to an EV comprising an IgG antibody or a fragment thereof that specifically binds human TfR.

19. The EV of any one of claims 6 to 18, wherein the single-domain antigen-binding moiety increases the permeability of the EV across the blood brain barrier by at least about 10%, at least about 20%, at least about 30%, at least about 40%, at least about 75%, at least about 100%, at least about 150%, at least about 200%, at least about 250%, at least about 300%, at least about 350%, at least about 400%, at least about 450%, at least about 500%, at least about 600%, at least about 700%, at least about 800%, at least about 900%, or at least about 1000%, as compared to an EV comprising an scFv that specifically binds human TfR.

20. The EV of any one of claims 1 to 19, wherein the antigen-binding moeity is present at a concentration of at least about 10 copies per EV, at least about 20 copies per EV, at least about 30 copies per EV, at least about 40 copies per EV, at least about 50 copies per EV, at least about 75 copies per EV, at least about 100 copies per EV, at least about 150 copies per EV, at least about 200 copies per EV, at least about 250 copies per EV, at least about 300 copies per EV, at least about 350 copies per EV, at least about 400 copies per EV, at least about 450 copies per EV, at least about 500 copies per EV, at least about 600 copies per EV, at least about 700 copies per EV, at least about 800 copies per EV, at least about 900 copies per EV, at least about 1000 copies per EV, at least about 1250 copies per EV, at least about 1500 copies per EV, at least about 2000 copies per EV, at least about 2500 copies per EV, at least about 3000 copies per EV, at least about 3500 copies per EV, at least about 4000 copies per EV, at least about 4500 copies per EV, or at least about 5000 copies per EV.

21. The EV of any one of claims 1 to 20, wherein the antigen-binding moeity is present at a concentration of at least about 500 copies per EV.

22. The EV of any one of claims 1 to 21, wherein the antigen-binding moeity is present at a concentration of at least about 1000 copies per EV.

23. The EV of any one of claims 1 to 22, wherein the antigen-binding moeity specifically binds human TfR with a K_{D} of less than 5×10⁻⁶, 2×10⁻⁶, 1×10⁻⁶, 5×10⁻⁷, 1×10⁻⁷, 5×10⁻⁸, 1×10⁻⁸, 5×10⁻⁹, or 1×10⁻⁹ M.

24. The EV of any one of claims 1 to 23, which further comprises an anti-phagocytic signal.

25. The EV of claim 24, wherein the anti-phagocytic signal is selected from CD47, CD24, a fragment thereof, and any combination thereof.

26. The EV of claim 24 or 25, wherein the anti-phagocytic signal is associated with the exterior surface of the EV.

27. The EV of any one of claims 1 to 26, which further comprises a biologically active moiety.

28. The EV of claim 27, wherein the biologically active moiety comprises a therapeutic molecule, immune modulator, adjuvant, or any combination thereof or a nucleic acid encoding the therapeutic molecule, immune modulator, adjuvant, or any combination thereof.

29. The EV of claim 28, wherein the nucleic acid encoding the therapeutic molecule, immune modulator, adjuvant, or any combination thereof comprises an mRNA, siRNA, shRNA, miRNA, or any combination thereof.

30. The EV of claim 28, wherein the therapeutic molecule comprises an antigen.

31. The EV of claim 28, wherein the adjuvant comprises a Stimulator of Interferon Genes (STING) agonist, a toll-like receptor (TLR) agonist, an inflammatory mediator, or any combination thereof.

32. The EV of claim 28 or 31, wherein the adjuvant comprises a STING agonist.

33. The EV of claim 31 or 32, wherein the STING agonist comprises a cyclic dinucleotide STING agonist or a non-cyclic dinucleotide STING agonist.

34. The EV of claim 28, wherein the adjuvant is a TLR agonist.

35. The EV of claim 34, wherein the TLR agonist comprises a TLR2 agonist (e.g., lipoteichoic acid, atypical LPS, MALP-2 and MALP-404, OspA, porin, LcrV, lipomannan, GPI anchor, lysophosphatidylserine, lipophosphoglycan (LPG), glycophosphatidylinositol (GPI), zymosan, hsp60, gH/gL glycoprotein, hemagglutinin), a TLR3 agonist (e.g., double-stranded RNA, e.g., poly(I:C)), a TLR4 agonist (e.g., lipopolysaccharides (LPS), lipoteichoic acid, β-defensin 2, fibronectin EDA, HMGB1, snapin, tenascin C), a TLR5 agonist (e.g., flagellin), a TLR6 agonist, a TLR7/8 agonist (e.g., single-stranded RNA, CpG-A, Poly G10, Poly G3, Resiquimod), a TLR9 agonist (e.g., unmethylated CpG DNA), or any combination thereof.

36. The EV of claim 28, wherein the immune modulator comprises a cytokine.

37. The EV of claim 36, wherein the cytokine comprises IL-12.

38. The EV of any one of claims 1 to 37, wherein the antigen-binding moeity, the biologically active moiety, and/or the anti-phagocytic signal are linked to the exterior surface of the EV by a scaffold protein.

39. The EV of claim 38, wherein the scaffold protein is a Scaffold X protein.

40. The EV of claim 39, wherein the Scaffold X protein comprises prostaglandin F2 receptor negative regulator (the PTGFRN protein); basigin (the BSG protein); immunoglobulin superfamily member 2 (the IGSF2 protein); immunoglobulin superfamily member 3 (the IGSF3 protein); immunoglobulin superfamily member 8 (the IGSF8 protein); integrin beta-1 (the ITGB1 protein); integrin alpha-4 (the ITGA4 protein); 4F2 cell-surface antigen heavy chain (the SLC3A2 protein); a class of ATP transporter proteins (the ATP1A1, ATP1A2, ATP1A3, ATP1A4, ATP1B3, ATP2B1, ATP2B2, ATP2B3, ATP2B4 proteins), CD13, aminopeptidase N (ANPEP), neprilysin (membrane metalloendopeptidase; MME), ectonucleotide pyrophosphatase/phosphodiesterase family member 1 (ENPP1), neuropilin-1 (NRP1), CD9, CD63, CD81, PDGFR, GPI anchor proteins, lactadherin, LAMP2, LAMP2B, a fragment thereof, or any combination thereof.

41. The EV of claim 39 or 40, wherein the Scaffold X protein comprises the amino acid sequence set forth as SEQ ID NO: 33.

42. The EV of claim 39 or 40, wherein the Scaffold X protein comprises an amino acid sequence having at least about 70%, at least about 75%, at least about 80%, at least about 85%, at least about 90%, at least about 95%, at least about 96%, at least about 97%, at least about 98%, at least about 99%, or about 100% sequence identity to SEQ ID NO: 1.

43. The EV of any one of claims 27 to 42, wherein the biologically active moiety is linked to the luminal surface of the EV by a scaffold protein.

44. The EV of claim 43, wherein the scaffold protein is a Scaffold Y protein.

45. The EV of claim 44, wherein the Scaffold Y protein comprises myristoylated alanine rich Protein Kinase C substrate (the MARCKS protein), myristoylated alanine rich Protein Kinase C substrate like 1 (the MARCKSL1 protein), brain acid soluble protein 1 (the BASP1 protein), a fragment thereof, and or any combination thereof.

46. The EV of claim 44 or 45, wherein the Scaffold Y protein is BASP1 protein or a fragment thereof.

47. The EV of any one of claims 44 to 46, wherein the Scaffold Y protein comprises an N-terminus domain (ND) and an effector domain (ED), wherein the ND and/or the ED are associated with the luminal surface of the EV.

48. The EV of claim 47, wherein the ND is associated with the luminal surface of the EV via myristoylation.

49. The EV of claim 47 or 48, wherein the ED is associated with the luminal surface of the EV by an ionic interaction.

50. The EV of any one of claims 47 to 49, wherein the ED comprises (i) a basic amino acid or (ii) two or more basic amino acids in sequence, wherein the basic amino acid is selected from the group consisting of Lys, Arg, His, and any combination thereof.

51. The EV of claim 50, wherein the basic amino acid is (Lys)n, wherein n is an integer between 1 and 10.

52. The EV of any one of claims 47 to 51, wherein the ED comprises Lys (K), KK, KKK, KKKK (SEQ ID NO: 205), KKKKK (SEQ ID NO: 206), Arg (R), RR, RRR, RRRR (SEQ ID NO: 207); RRRRR (SEQ ID NO: 208), KR, RK, KKR, KRK, RKK, KRR, RRK, (K/R)(K/R)(K/R)(K/R) (SEQ ID NO: 209), (K/R)(K/R)(K/R)(K/R)(K/R) (SEQ ID NO: 210, or any combination thereof.

53. The EV of any one of claims 47 to 52, wherein the ND comprises an amino acid sequence selected from the group consisting of (i) GGKLSKK (SEQ ID NO: 211), (ii) GAKLSKK (SEQ ID NO: 212), (iii) GGKQSKK (SEQ ID NO: 213), (iv) GGKLAKK (SEQ ID NO: 214, and (vi) any combination thereof.

54. The EV of any one of claims 43 to 53, wherein the scaffold protein comprises (i) GGKLSKKKKGYNVN (SEQ ID NO: 246), (ii) GAKLSKKKKGYNVN (SEQ ID NO: 247, (iii) GGKQSKKKKGYNVN (SEQ ID NO: 248), (iv) GGKLAKKKKGYNVN (SEQ ID NO: 249), (v) GGKLSKKKKGYSGG (SEQ ID NO: 250), (vi) GGKLSKKKKGSGGS (SEQ ID NO: 251), (vii) GGKLSKKKKSGGSG (SEQ ID NO: 252), (viii) GGKLSKKKSGGSGG (SEQ ID NO: 253), (ix) GGKLSKKSGGSGGS (SEQ ID NO: 254, (x) GGKLSKSGGSGGSV (SEQ ID NO: 255), or (xi) GAKKSKKRFSFKKS (SEQ ID NO: 256).

55. The EV of any one of claims 1 to 54, which is an exosome, a microvesicle, an apoptotic body, or any combination thereof.

56. The EV of any one of claims 1 to 55, which is an exosome.

57. A pharmaceutical composition comprising the EV of any one of claims 1 to 56 and a therapeutic molecule, an immune modulator, an adjuvant, or any combination thereof.

58. The pharmaceutical composition of claim 57, wherein the therapeutic molecule comprises an antigen.

59. The pharmaceutical composition of claim 57, wherein the adjuvant comprises a Stimulator of Interferon Genes (STING) agonist, a toll-like receptor (TLR) agonist, an inflammatory mediator, or any combination thereof.

60. The pharmaceutical composition of claim 57 or 59 wherein the adjuvant comprises a STING agonist.

61. The pharmaceutical composition of claim 60, wherein the STING agonist comprises a cyclic dinucleotide STING agonist or a non-cyclic dinucleotide STING agonist.

62. The pharmaceutical composition of claim 57 or 59, wherein the adjuvant is a TLR agonist.

63. The pharmaceutical composition of claim 62, wherein the TLR agonist comprises a TLR2 agonist (e.g., lipoteichoic acid, atypical LPS, MALP-2 and MALP-404, OspA, porin, LcrV, lipomannan, GPI anchor, lysophosphatidylserine, lipophosphoglycan (LPG), glycophosphatidylinositol (GPI), zymosan, hsp60, gH/gL glycoprotein, hemagglutinin), a TLR3 agonist (e.g., double-stranded RNA, e.g., poly(I:C)), a TLR4 agonist (e.g., lipopolysaccharides (LPS), lipoteichoic acid, β-defensin 2, fibronectin EDA, HMGB1, snapin, tenascin C), a TLR5 agonist (e.g., flagellin), a TLR6 agonist, a TLR7/8 agonist (e.g., single-stranded RNA, CpG-A, Poly G10, Poly G3, Resiquimod), a TLR9 agonist (e.g., unmethylated CpG DNA), or any combination thereof.

64. The pharmaceutical composition of any one of claims 57 to 63, wherein the therapeutic molecule, the immune modulator, the adjuvant, or any combination thereof, is associated with Scaffold X, Scaffold Y, or a combination thereof.

65. The pharmaceutical composition of any one of claims 57 to 64, wherein the immune modulator comprises a cytokine.

66. The pharmaceutical composition of claim 65, wherein the cytokine comprises an interferon.

67. The pharmaceutical composition of any one of claims 57 to 66, further comprising a pharmaceutically acceptable carrier.

68. A method of treating a disease or disorder in a subject in need thereof comprising administering to the subject the EV of any one of claims 1 to 56 or the pharmaceutical composition of any one of claims 46 to 56.

69. The method of claim 68, wherein the disease or disorder is a neurological disease or disorder.

70. The method of claim 68 or 69, wherein the neurological disease or disorder comprises a tumor.

71. The method of any one of claims 68 to 70, wherein the neurological disease or disorder comprises an acoustic neuroma, choroid plexus carcinoma, craniopharyngioma, embryonal tumor, glioma, medulloblastoma, meningioma, pediatric brain tumor, pineoblastoma, pituitary tumor, or a combination thereof.

72. The method of claim 71, wherein the glioma is selected from an ependymoma, astrocytoma, oligodendroglioma, brainstem glioma, optic nerve glioma, mixed glioma, oligoastrocytoma, or any combination thereof.

73. The method of claim 71, wherein the astrocytoma comprises glioblastoma multiforme (GBM).

74. The method of any one of claims 68 to 73, wherein the disease or disorder comprises a neoplastic meningitis, Parkinson disease, Alzheimer Disease, Huntington Disease, amyotrophic lateral sclerosis (ALS), or any combination thereof.

75. A method of targeting an extracellular vesicle (EV) to a cell of the central nervous system, comprising loading an antigen-binding moeity that specifically binds transferrin receptor (TfR) on the surface of the extracellular vesicle.

76. A method of increasing the permeability of an EV across the blood brain barrier in a human subject, comprising loading on the surface of the EV an antigen-binding moeity that specifically binds TfR.

77. The method of claim 75 or 76, wherein the antigen-binding moeity specifically binds human TfR.

78. The method of any one of claims 75 to 76, wherein the antigen-binding moeity increases the transport of the EV across the blood brain barrier in a human subject.

79. The method of claim 78, wherein the antigen-binding moeity increases the transport of the EV across the blood brain barrier by at least about 10%, at least about 20%, at least about 30%, at least about 40%, at least about 75%, at least about 100%, at least about 150%, at least about 200%, at least about 250%, at least about 300%, at least about 350%, at least about 400%, at least about 450%, at least about 500%, at least about 600%, at least about 700%, at least about 800%, at least about 900%, or at least about 1000%, as compared to a reference EV not comprising an antigen-binding moeity that specifically binds human TfR.

80. The method of any one of claims 75 to 79, wherein the antigen-binding moeity an antibody or an antigen-binding portion thereof.

81. The method of any one of claims 75 to 80, wherein the antigen-binding moeity comprises one or more single-domain antigen-binding moieties.

82. The method of claim 81, wherein the antigen-binding moeity comprises at least two single-domain antigen-binding moieties.

83. The method of claim 82, wherein the at least two single-domain antigen-binding moieties are the same.

84. The method of claim 82, wherein the at least two single-domain antigen-binding moieties are different.

85. The method of claim 84, wherein the at least two single-domain antigen-binding moieties comprise (i) a first single-domain antigen-binding moiety that binds a first epitope on TfR and (ii) a second single-domain antigen-binding moiety binds a second epitope on TfR.

86. The EV of any one of claims 82 to 85, wherein the antigen-binding moeity comprises at least three, at least four, at least five, or at least six single-domain antigen-binding moieties.

87. The method of any one of claims 81 to 86, wherein each of the one or more single-domain antigen-binding moieties are linked to each other by a linker.

88. The method of any one of claims 81 to 87, wherein the one or more single-domain antigen-binding moieties are selected from a VHH, a vNAR, an antigen-binding fragment of a VHH, an antigen-binding fragment of a vNAR, and any combination thereof.

89. The method of claim 88, wherein the vNAR or the fragment thereof is derived from an IgNAR.

90. The method of claim 88, wherein the vNAR or the fragment thereof is synthetic.

91. The method of claim 88, wherein the VHH or the fragment thereof is derived from a camelid antibody.

92. The method of claim 88, wherein the VHH or the fragment thereof is synthetic.


SEQUENCE LISTING (256 sequences)

SEQ ID NO: 1 - protein, 879 aa, Artificial Sequence
  The PTGFRN Protein
MGRLASRPLLLALLSLALCRGRVVRVPTATLVRVVGTELVIPCNVSDYDGPSEQNFDWSFSSLGSSFVELASTWEVGFPAQLYQERLQRGEILLRRTANDAVELHIKNVQPSDQGHYKCSTPSTDATVQGNYEDTVQVKVLADSLHVGPSARPPPSLSLREGEPFELRCTAASASPLHTHLALLWEVHRGPARRSVLALTHEGRFHPGLGYEQRYHSGDVRLDTVGSDAYRLSVSRALSADQGSYRCIVSEWIAEQGNWQEIQEKAVEVATVVIQPSVLRAAVPKNVSVAEGKELDLTCNITTDRADDVRPEVTWSFSRMPDSTLPGSRVLARLDRDSLVHSSPHVALSHVDARSYHLLVRDVSKENSGYYYCHVSLWAPGHNRSWHKVAEAVSSPAGVGVTWLEPDYQVYLNASKVPGFADDPTELACRVVDTKSGEANVRFTVSWYYRMNRRSDNVVTSELLAVMDGDWTLKYGERSKQRAQDGDFIFSKEHTDTFNFRIQRTTEEDRGNYYCVVSAWTKQRNNSWVKSKDVFSKPVNIFWALEDSVLVVKARQPKPFFAAGNTFEMTCKVSSKNIKSPRYSVLIMAEKPVGDLSSPNETKYIISLDQDSVVKLENWTDASRVDGVVLEKVQEDEFRYRMYQTQVSDAGLYRCMVTAWSPVRGSLWREAATSLSNPIEIDFQTSGPIFNASVHSDTPSVIRGDLIKLFCIITVEGAALDPDDMAFDVSWFAVHSFGLDKAPVLLSSLDRKGIVTTSRRDWKSDLSLERVSVLEFLLQVHGSEDQDFGNYYCSVTPWVKSPTGSWQKEAEIHSKPVFITVKMDVLNAFKYPLLIGVGLSTVIGLLSCLIGYCSSHWCCKKEVQETRRERRRLMSMEMD

SEQ ID NO: 2-8 - skipped (7 entries)

SEQ ID NO: 9 - protein, 385 aa, Artificial Sequence
  The BSG protein
MAAALFVLLGFALLGTHGASGAAGFVQAPLSQQRWVGGSVELHCEAVGSPVPEIQWWFEGQGPNDTCSQLWDGARLDRVHIHATYHQHAASTISIDTLVEEDTGTYECRASNDPDRNHLTRAPRVKWVRAQAVVLVLEPGTVFTTVEDLGSKILLTCSLNDSATEVTGHRWLKGGVVLKEDALPGQKTEFKVDSDDQWGEYSCVFLPEPMGTANIQLHGPPRVKAVKSSEHINEGETAMLVCKSESVPPVTDWAWYKITDSEDKALMNGSESRFFVSSSQGRSELHIENLNMEADPGQYRCNGTSSKGSDQAIITLRVRSHLAALWPFLGIVAEVLVLVTIIFIYEKRRKPEDVLDDDDAGSAPLKSSGQHQNDKGKNVRQRNSS

SEQ ID NO: 10-13 - skipped (4 entries)

SEQ ID NO: 14 - protein, 613 aa, Artificial Sequence
  The IGSF8 protein
MGALRPTLLPPSLPLLLLLMLGMGCWAREVLVPEGPLYRVAGTAVSISCNVTGYEGPAQQNFEWFLYRPEAPDTALGIVSTKDTQFSYAVFKSRVVAGEVQVQRLQGDAVVLKIARLQAQDAGIYECHTPSTDTRYLGSYSGKVELRVLPDVLQVSAAPPGPRGRQAPTSPPRMTVHEGQELALGCLARTSTQKHTHLAVSFGRSVPEAPVGRSTLQEVVGIRSDLAVEAGAPYAERLAAGELRLGKEGTDRYRMVVGGAQAGDAGTYHCTAAEWIQDPDGSWAQIAEKRAVLAHVDVQTLSSQLAVTVGPGERRIGPGEPLELLCNVSGALPPAGRHAAYSVGWEMAPAGAPGPGRLVAQLDTEGVGSLGPGYEGRHIAMEKVASRTYRLRLEAARPGDAGTYRCLAKAYVRGSGTRLREAASARSRPLPVHVREEGVVLEAVAWLAGGTVYRGETASLLCNISVRGGPPGLRLAASWWVERPEDGELSSVPAQLVGGVGQDGVAELGVRPGGGPVSVELVGPRSHRLRLHSLGPEDEGVYHCAPSAWVQHADYSWYQAGSARSGPVTVYPYMHALDTLFVPLLVGTGVALVTGATVLGTITCCFMKRLRKR

SEQ ID NO: 15-20 - skipped (6 entries)

SEQ ID NO: 21 - protein, 748 aa, Artificial Sequence
  The ITGB1 protein
MNLQPIFWIGLISSVCCVFAQTDENRCLKANAKSCGECIQAGPNCGWCTNSTFLQEGMPTSARCDDLEALKKKGCPPDDIENPRGSKDIKKNKNVTNRSKGTAEKLKPEDITQIQPQQLVLRLRSGEPQTFTLKFKRAEDYPIDLYYLMDLSYSMKDDLENVKSLGTDLMNEMRRITSDFRIGFGSFVEKTVMPYISTTPAKLRNPCTSEQNCTSPFSYKNVLSLTNKGEVFNELVGKQRISGNLDSPEGGFDAIMQVAVCGSLIGWRNVTRLLVFSTDAGFHFAGDGKLGGIVLPNDGQCHLENNMYTMSHYYDYPSIAHLVQKLSENNIQTIFAVTEEFQPVYKELKNLIPKSAVGTLSANSSNVIQLIIDAYNSLSSEVILENGKLSEGVTISYKSYCKNGVNGTGENGRKCSNISIGDEVQFEISITSNKCPKKDSDSFKIRPLGFTEEVEVILQYICECECQSEGIPESPKCHEGNGTFECGACRCNEGRVGRHCECSTDEVNSEDMDAYCRKENSSEICSNNGECVCGQCVCRKRDNTNEIYSGASNGQICNGRGICECGVCKCTDPKFQGQTCEMCQTCLGVCAEHKECVQCRAFNKGEKKDTCTQECSYFNITKVESRDKLPQPVQPDPVSHCKEKDVDDCWFYFTYSVNGNNEVMVHVVENPECPTGPDIIPIVAGVVAGIVLIGLALLLIWKLLMIIHDRREFAKFEKEKMNAKWDTGENPIYKSAVTTVVNPKYEGK

SEQ ID NO: 22 - protein, 1032 aa, Artificial Sequence [sequence omitted]

SEQ ID NO: 23 - protein, 630 aa, Artificial Sequence
  The SLC3A2 Protein, wherethe first Met is processed.
MELQPPEASIAVVSIPRQLPGSHSEAGVQGLSAGDDSELGSHCVAQTGLELLASGDPLPSASQNAEMIETGSDCVTQAGLQLLASSDPPALASKNAEVTGTMSQDTEVDMKEVELNELEPEKQPMNAASGAAMSLAGAEKNGLVKIKVAEDEAEAAAAAKFTGLSKEELLKVAGSPGWVRTRWALLLLFWLGWLGMLAGAVVIIVRAPRCRELPAQKWWHTGALYRIGDLQAFQGHGAGNLAGLKGRLDYLSSLKVKGLVLGPIHKNQKDDVAQTDLLQIDPNFGSKEDFDSLLQSAKKKSIRVILDLTPNYRGENSWFSTQVDTVATKVKDALEFWLQAGVDGFQVRDIENLKDASSFLAEWQNITKGFSEDRLLIAGTNSSDLQQILSLLESNKDLLLTSSYLSDSGSTGEHTKSLVTQYLNATGNRWCSWSLSQARLLTSFLPAQLLRLYQLMLFTLPGTPVFSYGDEIGLDAAALPGQPMEAPVMLWDESSFPDIPGAVSANMTVKGQSEDPGSLLSLFRRLSDQRSKERSLLHGDFHAFSAGPGLFSYIRHWDQNERFLVVLNFGDVGLSAGLQASDLPASASLPAKADLLLSTQPGREEGSPLELERLKLEPHEGLLLRFPYAA

SEQ ID NO: 24-32 - skipped (9 entries)

SEQ ID NO: 33 - protein, 192 aa, Artificial Sequence
  The PTGFRN protein Fragment
GPIFNASVHSDTPSVIRGDLIKLFCIITVEGAALDPDDMAFDVSWFAVHSFGLDKAPVLLSSLDRKGIVTTSRRDWKSDLSLERVSVLEFLLQVHGSEDQDFGNYYCSVTPWVKSPTGSWQKEAEIHSKPVFITVKMDVLNAFKYPLLIGVGLSTVIGLLSCLIGYCSSHWCCKKEVQETRRERRRLMSMEM

SEQ ID NO: 34-46 - skipped (13 entries)

SEQ ID NO: 47 - protein, 332 aa, Artificial Sequence
  The MARCKS protein
MGAQFSKTAAKGEAAAERPGEAAVASSPSKANGQENGHVKVNGDASPAAAESGAKEELQANGSAPAADKEEPAAAGSGAASPSAAEKGEPAAAAAPEAGASPVEKEAPAEGEAAEPGSPTAAEGEAASAASSTSSPKAEDGATPSPSNETPKKKKKRFSFKKSFKLSGFSFKKNKKEAGEGGEAEAPAAEGGKDEAAGGAAAAAAEAGAASGEQAAAPGEEAAAGEEGAAGGDPQEAKPQEAAVAPEKPPASDETKAAEEPSKVEEKKAEEAGASAAACEAPSAAGPGAPPEQEAAPAEEPAAAAASSACAAPSQEAQPECSPEAPPAEAAE

SEQ ID NO: 48 - protein, 195 aa, Artificial Sequence
  The MARCKSL1 protein
MGSQSSKAPRGDVTAEEAAGASPAKANGQENGHVKSNGDLSPKGEGESPPVNGTDEAAGATGDAIEPAPPSQGAEAKGEVPPKETPKKKKKFSFKKPFKLSGLSFKRNRKEGGGDSSASSPTEEEQEQGEIGACSDEGTAQEGKAAATPESQEPQAKGAEASAASEEEAGPQATEPSTPSGPESGPTPASAEQNE

SEQ ID NO: 49 - protein, 227 aa, Artificial Sequence
  The BASP1 protein
MGGKLSKKKKGYNVNDEKAKEKDKKAEGAATEEEGTPKESEPQAAAEPAEAKEGKEKPDQDAEGKAEEKEGEKDAAAAKEEAPKAEPEKTEGAAEAKAEPPKAPEQEQAAPGPAAGGEAPKAAEAAAAPAESAAPAAGEEPSKEEGEPKKTEAPAAPAAQETKSDGAPASDSKPGSSEAAPSSKETPAATEAPSSTPKAQGPAASAEEPKPVEAPAANSDQTVTVKE

SEQ ID NO: 50-145 - skipped (96 entries)

SEQ ID NO: 146 - protein, 17 aa, Artificial Sequence
  a PMP22 ASO
CTCATTCGCGTTTCCGC

SEQ ID NO: 147-155 - skipped (9 entries)

SEQ ID NO: 156 - protein, 323 aa, Artificial Sequence
  Canonical CD47
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 157 - protein, 292 aa, Artificial Sequence
  CD47 HUMAN Isoform OA3-293
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFV

SEQ ID NO: 158 - protein, 305 aa, Artificial Sequence
  CD47 HUMAN Isoform OA3-305
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRNN

SEQ ID NO: 159 - protein, 311 aa, Artificial Sequence
  CD47 HUMAN Isoform OA3-312
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLN

SEQ ID NO: 160 - protein, 21 aa, Artificial Sequence
  Minimal “self” peptide
GNYTCEVTELTREGETIIELK

SEQ ID NO: 161 - protein, 119 aa, Artificial Sequence
  BDNF
HSDPARRGELSVCDSISEWVTAADKKTAVDMSGGTVTVLEKVPVSKGQLKQYFYETKCNPMGYTKEGCRGIDKRHWNSQCRTTQSYVRALTMDSKKRIGWRFIRIDTSCVCTLTIKRGR

SEQ ID NO: 162 - protein, 120 aa, Artificial Sequence
  beta NGF
SSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKAVRRA

SEQ ID NO: 163 - protein, 119 aa, Artificial Sequence [sequence omitted]

SEQ ID NO: 164 - protein, 130 aa, Artificial Sequence
  NT-4
GVSETAPASRRGELAVCDAVSGWVTDRRTAVDLRGREVEVLGEVPAAGGSPLRQYFFETRCKADNAEEGGPGAGGGGCRGVDRRHWVSECKAKQSYVRALTADAQGRVGWRWIRIDTACVCTLLSRTGRA

SEQ ID NO: 165 - protein, 29 aa, Artificial Sequence
  RVG
YTIWMPENPRPGTPCDIFTNSRGKRASNG

SEQ ID NO: 166 - protein, 14 aa, Artificial Sequence
  TAxI peptide
SACQSQSQMRCGGG

SEQ ID NO: 167 - protein, 7 aa, Artificial Sequence
  TAxI transport peptide
QSQSQMR

SEQ ID NO: 168 - protein, 7 aa, Artificial Sequence
  TAxI transport peptide
ASGAQAR

SEQ ID NO: 169 - protein, 14 aa, Artificial Sequence
  TAxI transport peptide
TSTAPHLRLRLTSR

SEQ ID NO: 170 - protein, 7 aa, Artificial Sequence
  Nuclear localizing signal (NLS)
PPKKRKV

SEQ ID NO: 171 - protein, 6 aa, Artificial Sequence
  Nuclear localizing signal (NLS)
PKKRKV

SEQ ID NO: 172 - protein, 19 aa, Artificial Sequence
  Angiopep-2
TFFYGGSRGKRNNFKTEEY

SEQ ID NO: 173 - protein, 39 aa, Artificial Sequence
  ApoB (3371-3409)
SSVIDALQYKLEGTTRLTRKRGLKLATALSLSNKFVEGS

SEQ ID NO: 174 - protein, 18 aa, Artificial Sequence
  ApoE (159-167)2
LRKLRKRLLLRKLRKRLL

SEQ ID NO: 175 - protein, 8 aa, Artificial Sequence
  Peptide-22
CMPRLRGC

SEQ ID NO: 176 - protein, 12 aa, Artificial Sequence
  THR
THRPPMWSPVWP

SEQ ID NO: 177 - protein, 12 aa, Artificial Sequence
  THR retro-enantio
PWVPSWMPPRHT

SEQ ID NO: 178 - protein, 9 aa, Artificial Sequence
  CRT
CRTIGPSVC

SEQ ID NO: 179 - protein, 30 aa, Artificial Sequence
  Leptin30
YQQILTSMPSRNVIQISNDLENLRDLLHVL

SEQ ID NO: 180 - protein, 29 aa, Artificial Sequence
  RVG29
YTIWMPENPRPGTPCDIFTNSRGKRASNG

SEQ ID NO: 181 - protein, 16 aa, Artificial Sequence
  DCDX
GREIRTGRAERWSEKF

SEQ ID NO: 182 - protein, 18 aa, Artificial Sequence
  Apamin
CNCKAPETALCARRCQQH

SEQ ID NO: 183 - protein, 8 aa, Artificial Sequence
  MiniAp-4
KAPETALD

SEQ ID NO: 184 - protein, 2 aa, Artificial Sequence
  GSH
CG

SEQ ID NO: 185 - protein, 13 aa, Artificial Sequence
  G23
HLNILSTLWKYRC

SEQ ID NO: 186 - protein, 7 aa, Artificial Sequence
  g7
GFTGFLS

SEQ ID NO: 187 - protein, 12 aa, Artificial Sequence
  TGN
TGNYKALHPHNG

SEQ ID NO: 188 - protein, 11 aa, Artificial Sequence
  TAT (47-57)
YGRKKRRQRRR

SEQ ID NO: 189 - protein, 18 aa, Artificial Sequence
  SynB1
RGGRLSYSRRRFSTSTGR

SEQ ID NO: 190 - protein, 4 aa, Artificial Sequence
  Diketopiperazines
MFMF

SEQ ID NO: 191 - protein, 4 aa, Artificial Sequence
  PhPro
PPPP

SEQ ID NO: 192 - protein, 29 aa, Artificial Sequence
  Linker
GGSSGSGSGSGGGGSGGGGTGTSSSGTGT

SEQ ID NO: 193 - protein, 111 aa, Artificial Sequence
  vNAR-anti-TfnR-1
ARVDQTPQTITKETGESLTINCVLRDNDCTLSSTHWYRKKSGSTNEERISKGGRYVETVNSGSKSFSLRINDLTVEDSGTYRCNVYAMTRNWWCDVYGGGTVVTVNAASGA

SEQ ID NO: 194 - protein, 113 aa, Artificial Sequence
  vNAR- anti-TfnR-2
ARVDQTPQTITKETGESLTINCVLRDSNCALSSTYWYRKKSGSTKEESISKGGRYVETVNSGSKSFSLRINDLTVEDSGTYRCNVWHDLVWSVCTTDVYGGGTVVTVNAASGA

SEQ ID NO: 195 - protein, 116 aa, Artificial Sequence
  vNAR- anti-TfnR-3
ARVDQTPQTITKEEGESLTINCVLRDSSSALASTSWYRKKSGSTREETISKGGRYVETVNSGSKSFSLRINDLTVEDSGTYRCNVYELVEDTSAYEIGVDVYGDGTAVTVNAASGA

SEQ ID NO: 196-204 - skipped (9 entries)

SEQ ID NO: 205 - protein, 4 aa, Artificial Sequence
  Effector domain (ED)
KKKK

SEQ ID NO: 206 - protein, 5 aa, Artificial Sequence
  Effector domain (ED)
KKKKK

SEQ ID NO: 207 - protein, 4 aa, Artificial Sequence
  Effector domain (ED)
RRRR

SEQ ID NO: 208 - protein, 5 aa, Artificial Sequence
  Effector domain (ED)
RRRRR

SEQ ID NO: 209 - protein, 4 aa, Artificial Sequence
  Effector domain (ED)
  MISC_FEATURE (1)..(4): Wherein Xaa can be either Lys or Arg
XXXX

SEQ ID NO: 210 - protein, 5 aa, Artificial Sequence
  Effector domain (ED)
  MISC_FEATURE (1)..(5): Wherein Xaa can be either Lys or Arg
XXXXX

SEQ ID NO: 211 - protein, 7 aa, Artificial Sequence
  N-terminus domain (ND)
GGKLSKK

SEQ ID NO: 212 - protein, 7 aa, Artificial Sequence
  N-terminus domain (ND)
GAKLSKK

SEQ ID NO: 213 - protein, 7 aa, Artificial Sequence
  N-terminus domain (ND)
GGKQSKK

SEQ ID NO: 214 - protein, 7 aa, Artificial Sequence
  N-terminus domain (ND)
GGKLAKK

SEQ ID NO: 215-245 - skipped (31 entries)

SEQ ID NO: 246 - protein, 14 aa, Artificial Sequence
  Scaffold protein
GGKLSKKKKGYNVN

SEQ ID NO: 247 - protein, 14 aa, Artificial Sequence
  Scaffold protein
GAKLSKKKKGYNVN

SEQ ID NO: 248 - protein, 14 aa, Artificial Sequence
  Scaffold protein
GGKQSKKKKGYNVN

SEQ ID NO: 249 - protein, 14 aa, Artificial Sequence
  Scaffold protein
GGKLAKKKKGYNVN

SEQ ID NO: 250 - protein, 14 aa, Artificial Sequence
  Scaffold protein
GGKLSKKKKGYSGG

SEQ ID NO: 251 - protein, 14 aa, Artificial Sequence
  Scaffold protein
GGKLSKKKKGSGGS

SEQ ID NO: 252 - protein, 14 aa, Artificial Sequence
  Scaffold protein
GGKLSKKKKSGGSG

SEQ ID NO: 253 - protein, 14 aa, Artificial Sequence
  Scaffold protein
GGKLSKKKSGGSGG

SEQ ID NO: 254 - protein, 14 aa, Artificial Sequence
  Scaffold protein
GGKLSKKSGGSGGS

SEQ ID NO: 255 - protein, 14 aa, Artificial Sequence
  Scaffold protein
GGKLSKSGGSGGSV

SEQ ID NO: 256 - protein, 14 aa, Artificial Sequence
  Scaffold protein
GAKKSKKRFSFKKS